SEQUENCE LISTING (133 sequences)

SEQ ID NO: 1 - protein, 5 aa, synthetic construct
RYWMN

SEQ ID NO: 2 - protein, 17 aa, synthetic construct
EITPDSSTINYAPSLKG

SEQ ID NO: 3 - protein, 10 aa, synthetic construct
PYDYGAWFAS

SEQ ID NO: 4 - protein, 14 aa, synthetic construct
RSSTGAVTTSNYAN

SEQ ID NO: 5 - protein, 7 aa, synthetic construct
GTNKRAP

SEQ ID NO: 6 - protein, 9 aa, synthetic construct
ALWYSNHWV

SEQ ID NO: 7 - protein, 476 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSLKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 119 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSS

SEQ ID NO: 9 - protein, 109 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 10 - protein, 248 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 11 - protein, 21 aa, synthetic construct
IYIWAPLAGTCGVLLLSLVIT

SEQ ID NO: 12 - protein, 42 aa, synthetic construct
KRGRKKLLYIFKQPFMRPVQTTQEEDGCSCRFPEEEEGGCEL

SEQ ID NO: 13 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 222 aa, synthetic construct
KPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 238 aa, synthetic construct
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 16 - protein, 20 aa, synthetic construct
GGGGSGGGGSGGGGSGGGGS

SEQ ID NO: 17 - protein, 5 aa, synthetic construct
GGGGS

SEQ ID NO: 18 - protein, 19 aa, synthetic construct
GEGRGSLLTCGDVEENPGP

SEQ ID NO: 19 - protein, 47 aa, synthetic construct
KPTTTPAPRPPTPAPTIASQPLSLRPEACRPAAGGAVHTRGLDFACD

SEQ ID NO: 20 - DNA, 1428 bp, synthetic construct
  source 1..1428: mol_type = other DNA
gaggtgcagctggtggagagcggcggcggcctggtgcagcccggcggcagcctgaggctgagctgcgccgccagcggcttcaccttcagcaggtactggatgaactgggtgaggcaggcccccggcaagggcctggagtgggtgggcgagatcacccccgacagcagcaccatcaactacgcccccagcctgaagggcaggttcaccatcagcagggacaacgccaagaacagcctgtacctgcagatgaacagcctgagggccgaggacaccgccgtgtactactgcgccaggccctacgactacggcgcctggttcgccagctggggccagggcaccctggtgaccgtgagcagcggagggggcggaagtggtggcgggggaagcggcgggggtggcagcggagggggcggatctcaggccgtggtgacccaggagcccagcctgaccgtgagccccggcggcaccgtgaccctgacctgcaggagcagcaccggcgccgtgaccaccagcaactacgccaactgggtgcaggagaagcccgaccacctgttcaccggcctgatcggcggcaccaacaagagggcccccggcacccccgccaggttcagcggcagcctgctgggcggcaaggccgccctgaccctgagcggcgcccagcccgaggacgaggccgagtactactgcgccctgtggtacagcaaccactgggtgttcggcggcggcaccaagctgaccgtcctaggagggggcggatccttgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 357 bp, synthetic construct
  source 1..357: mol_type = other DNA
gaggtgcagctggtggagagcggcggcggcctggtgcagcccggcggcagcctgaggctgagctgcgccgccagcggcttcaccttcagcaggtactggatgaactgggtgaggcaggcccccggcaagggcctggagtgggtgggcgagatcacccccgacagcagcaccatcaactacgcccccagcctgaagggcaggttcaccatcagcagggacaacgccaagaacagcctgtacctgcagatgaacagcctgagggccgaggacaccgccgtgtactactgcgccaggccctacgactacggcgcctggttcgccagctggggccagggcaccctggtgaccgtgagcagc

SEQ ID NO: 22 - DNA, 327 bp, synthetic construct
  source 1..327: mol_type = other DNA
caggccgtggtgacccaggagcccagcctgaccgtgagccccggcggcaccgtgaccctgacctgcaggagcagcaccggcgccgtgaccaccagcaactacgccaactgggtgcaggagaagcccgaccacctgttcaccggcctgatcggcggcaccaacaagagggcccccggcacccccgccaggttcagcggcagcctgctgggcggcaaggccgccctgaccctgagcggcgcccagcccgaggacgaggccgagtactactgcgccctgtggtacagcaaccactgggtgttcggcggcggcaccaagctgaccgtccta

SEQ ID NO: 23 - DNA, 744 bp, synthetic construct
  source 1..744: mol_type = other DNA
gaggtgcagctggtggagagcggcggcggcctggtgcagcccggcggcagcctgaggctgagctgcgccgccagcggcttcaccttcagcaggtactggatgaactgggtgaggcaggcccccggcaagggcctggagtgggtgggcgagatcacccccgacagcagcaccatcaactacgcccccagcctgaagggcaggttcaccatcagcagggacaacgccaagaacagcctgtacctgcagatgaacagcctgagggccgaggacaccgccgtgtactactgcgccaggccctacgactacggcgcctggttcgccagctggggccagggcaccctggtgaccgtgagcagcggagggggcggaagtggtggcgggggaagcggcgggggtggcagcggagggggcggatctcaggccgtggtgacccaggagcccagcctgaccgtgagccccggcggcaccgtgaccctgacctgcaggagcagcaccggcgccgtgaccaccagcaactacgccaactgggtgcaggagaagcccgaccacctgttcaccggcctgatcggcggcaccaacaagagggcccccggcacccccgccaggttcagcggcagcctgctgggcggcaaggccgccctgaccctgagcggcgcccagcccgaggacgaggccgagtactactgcgccctgtggtacagcaaccactgggtgttcggcggcggcaccaagctgaccgtccta

SEQ ID NO: 24 - DNA, 63 bp, synthetic construct
  source 1..63: mol_type = other DNA
atctacatctgggcgcccctggccgggacttgtggggtccttctcctgtcactggttatcacc

SEQ ID NO: 25 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 26 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 27 - DNA, 525 bp, synthetic construct
  source 1..525: mol_type = other DNA
atctacatctgggcgcccctggccgggacttgtggggtccttctcctgtcactggttatcaccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 28 - DNA, 63 bp, synthetic construct
  source 1..63: mol_type = other DNA
tccggagagggcagaggaagtcttctaacatgcggtgacgtggaggagaatcccggccctagg

SEQ ID NO: 29 - DNA, 717 bp, synthetic construct
  source 1..717: mol_type = other DNA
gtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtga

SEQ ID NO: 30 - DNA, 2211 bp, synthetic construct
  source 1..2211: mol_type = other DNA
gaggtgcagctggtggagagcggcggcggcctggtgcagcccggcggcagcctgaggctgagctgcgccgccagcggcttcaccttcagcaggtactggatgaactgggtgaggcaggcccccggcaagggcctggagtgggtgggcgagatcacccccgacagcagcaccatcaactacgcccccagcctgaagggcaggttcaccatcagcagggacaacgccaagaacagcctgtacctgcagatgaacagcctgagggccgaggacaccgccgtgtactactgcgccaggccctacgactacggcgcctggttcgccagctggggccagggcaccctggtgaccgtgagcagcggagggggcggaagtggtggcgggggaagcggcgggggtggcagcggagggggcggatctcaggccgtggtgacccaggagcccagcctgaccgtgagccccggcggcaccgtgaccctgacctgcaggagcagcaccggcgccgtgaccaccagcaactacgccaactgggtgcaggagaagcccgaccacctgttcaccggcctgatcggcggcaccaacaagagggcccccggcacccccgccaggttcagcggcagcctgctgggcggcaaggccgccctgaccctgagcggcgcccagcccgaggacgaggccgagtactactgcgccctgtggtacagcaaccactgggtgttcggcggcggcaccaagctgaccgtcctaggagggggcggatccttgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgaattctccggagagggcagaggaagtcttctaacatgcggtgacgtggaggagaatcccggccctagggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 31 - protein, 266 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSGGGGSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSGGGGSLKPTTTPAPRPPT

SEQ ID NO: 32 - protein, 271 aa, synthetic construct
MLLLVTSLLLCELPHPAFLLIPAQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSGGGGSGGGGSGGGGSEVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSS

SEQ ID NO: 33 - DNA, 1428 bp, synthetic construct
  source 1..1428: mol_type = other DNA
caggccgtggtgacccaggagcccagcctgaccgtgagccccggcggcaccgtgaccctgacctgcaggagcagcaccggcgccgtgaccaccagcaactacgccaactgggtgcaggagaagcccgaccacctgttcaccggcctgatcggcggcaccaacaagagggcccccggcacccccgccaggttcagcggcagcctgctgggcggcaaggccgccctgaccctgagcggcgcccagcccgaggacgaggccgagtactactgcgccctgtggtacagcaaccactgggtgttcggcggcggcaccaagctgaccgtcctaggagggggcggaagtggtggcgggggaagcggcgggggtggcagcggagggggcggatctgaggtgcagctggtggagagcggcggcggcctggtgcagcccggcggcagcctgaggctgagctgcgccgccagcggcttcaccttcagcaggtactggatgaactgggtgaggcaggcccccggcaagggcctggagtgggtgggcgagatcacccccgacagcagcaccatcaactacgcccccagcctgaagggcaggttcaccatcagcagggacaacgccaagaacagcctgtacctgcagatgaacagcctgagggccgaggacaccgccgtgtactactgcgccaggccctacgactacggcgcctggttcgccagctggggccagggcaccctggtgaccgtgagcagcggagggggcggatccttgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 34 - DNA, 2211 bp, synthetic construct
  source 1..2211: mol_type = other DNA
caggccgtggtgacccaggagcccagcctgaccgtgagccccggcggcaccgtgaccctgacctgcaggagcagcaccggcgccgtgaccaccagcaactacgccaactgggtgcaggagaagcccgaccacctgttcaccggcctgatcggcggcaccaacaagagggcccccggcacccccgccaggttcagcggcagcctgctgggcggcaaggccgccctgaccctgagcggcgcccagcccgaggacgaggccgagtactactgcgccctgtggtacagcaaccactgggtgttcggcggcggcaccaagctgaccgtcctaggagggggcggaagtggtggcgggggaagcggcgggggtggcagcggagggggcggatctgaggtgcagctggtggagagcggcggcggcctggtgcagcccggcggcagcctgaggctgagctgcgccgccagcggcttcaccttcagcaggtactggatgaactgggtgaggcaggcccccggcaagggcctggagtgggtgggcgagatcacccccgacagcagcaccatcaactacgcccccagcctgaagggcaggttcaccatcagcagggacaacgccaagaacagcctgtacctgcagatgaacagcctgagggccgaggacaccgccgtgtactactgcgccaggccctacgactacggcgcctggttcgccagctggggccagggcaccctggtgaccgtgagcagcggagggggcggatccttgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgaattctccggagagggcagaggaagtcttctaacatgcggtgacgtggaggagaatcccggccctagggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 35 - protein, 17 aa, synthetic construct
EITPDSSTINYTPSLKD

SEQ ID NO: 36 - protein, 119 aa, synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSA

SEQ ID NO: 37 - protein, 109 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNHWVFGGGTKLTVL

SEQ ID NO: 38 - protein, 447 aa, synthetic construct
EVKLLESGGGLVQPGGSLKLSCAASGFDFSRYWMNWVRQAPGKGLEWIGEITPDSSTINYTPSLKDKFIISRDNAKNTLYLQMIKVRSEDTALYYCVRPYDYGAWFASWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 39 - protein, 215 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 40 - protein, 17 aa, synthetic construct
EITPDSSTINYTPSLKG

SEQ ID NO: 41 - protein, 119 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSS

SEQ ID NO: 42 - protein, 447 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 43 - protein, 215 aa, synthetic construct
QAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 44 - protein, 119 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSS

SEQ ID NO: 45 - protein, 447 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 46 - protein, 447 aa, synthetic construct
EVQLVESGGGLVQPGGSLRLSCAASGFTFSRYWMNWVRQAPGKGLEWVGEITPDSSTINYAPSLKGRFTISRDNAKNSLYLQMNSLRAEDTAVYYCARPYDYGAWFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 47 - protein, 17 aa, synthetic construct
EITPDSSTINYADSVKG

SEQ ID NO: 48 - protein, 119 aa, synthetic construct
EVQLVESGGGLVQPGGSLRLSCAASGFDFSRYWMNWVRQAPGKGLEWVSEITPDSSTINYADSVKGRFTISRDNAKNSLYLQMNSLRAEDTAVYYCARPYDYGAWFASWGQGTLVTVSS

SEQ ID NO: 49 - protein, 447 aa, synthetic construct
EVQLVESGGGLVQPGGSLRLSCAASGFDFSRYWMNWVRQAPGKGLEWVSEITPDSSTINYADSVKGRFTISRDNAKNSLYLQMNSLRAEDTAVYYCARPYDYGAWFASWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 50 - protein, 109 aa, synthetic construct
QAVVTQEPSLTVSPGGTVTLTCRSSTGAVTTSNYANWFQQKPGQAFTGLIGGTNKRAPGTPARFSGSLLGGKAALTLSGAQPEDEAEYYCALWYSNHWVFGGGTKLTVL

SEQ ID NO: 51 - protein, 215 aa, synthetic construct
QAVVTQEPSLTVSPGGTVTLTCRSSTGAVTTSNYANWFQQKPGQAFTGLIGGTNKRAPGTPARFSGSLLGGKAALTLSGAQPEDEAEYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 52 - protein, 14 aa, synthetic construct
GSSTGAVTTSNYAN

SEQ ID NO: 53 - protein, 109 aa, synthetic construct
QAVVTQEPSLTVSPGGTVTLTCGSSTGAVTTSNYANWFQQKPGQAPRTLIGGTNKRAPGTPARFSGSLLGGKAALTLSGAQPEDEAEYYCALWYSNHWVFGGGTKLTVL

SEQ ID NO: 54 - protein, 215 aa, synthetic construct
QAVVTQEPSLTVSPGGTVTLTCGSSTGAVTTSNYANWFQQKPGQAPRTLIGGTNKRAPGTPARFSGSLLGGKAALTLSGAQPEDEAEYYCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 55 - protein, 230 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 56 - DNA, 780 bp, Homo sapiens
  source 1..780: mol_type = other DNA
atggcgcgcccgcatccgtggtggctgtgcgtgctgggcaccctggtgggcctgagcgcgaccccggcgccgaaaagctgcccggaacgccattattgggcgcagggcaaactgtgctgccagatgtgcgaaccgggcacctttctggtgaaagattgcgatcagcatcgcaaagcggcgcagtgcgatccgtgcattccgggcgtgagctttagcccggatcatcatacccgcccgcattgcgaaagctgccgccattgcaacagcggcctgctggtgcgcaactgcaccattaccgcgaacgcggaatgcgcgtgccgcaacggctggcagtgccgcgataaagaatgcaccgaatgcgatccgctgccgaacccgagcctgaccgcgcgcagcagccaggcgctgagcccgcatccgcagccgacccatctgccgtatgtgagcgaaatgctggaagcgcgcaccgcgggccatatgcagaccctggcggattttcgccagctgccggcgcgcaccctgagcacccattggccgccgcagcgcagcctgtgcagcagcgattttattcgcattctggtgatttttagcggcatgtttctggtgtttaccctggcgggcgcgctgtttctgcatcagcgccgcaaatatcgcagcaacaaaggcgaaagcccggtggaaccggcggaaccgtgccattatagctgcccgcgcgaagaagaaggcagcaccattccgattcaggaagattatcgcaaaccggaaccggcgtgcagcccg

SEQ ID NO: 57 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 58 - DNA, 750 bp, Mus musculus
  source 1..750: mol_type = other DNA
atggcgtggccgccgccgtattggctgtgcatgctgggcaccctggtgggcctgagcgcgaccctggcgccgaacagctgcccggataaacattattggaccggcggcggcctgtgctgccgcatgtgcgaaccgggcaccttttttgtgaaagattgcgaacaggatcgcaccgcggcgcagtgcgatccgtgcattccgggcaccagctttagcccggattatcatacccgcccgcattgcgaaagctgccgccattgcaacagcggctttctgattcgcaactgcaccgtgaccgcgaacgcggaatgcagctgcagcaaaaactggcagtgccgcgatcaggaatgcaccgaatgcgatccgccgctgaacccggcgctgacccgccagccgagcgaaaccccgagcccgcagccgccgccgacccatctgccgcatggcaccgaaaaaccgagctggccgctgcatcgccagctgccgaacagcaccgtgtatagccagcgcagcagccatcgcccgctgtgcagcagcgattgcattcgcatttttgtgacctttagcagcatgtttctgatttttgtgctgggcgcgattctgttttttcatcagcgccgcaaccatggcccgaacgaagatcgccaggcggtgccggaagaaccgtgcccgtatagctgcccgcgcgaagaagaaggcagcgcgattccgattcaggaagattatcgcaaaccggaaccggcgttttatccg

SEQ ID NO: 59 - protein, 250 aa, Mus musculus
MAWPPPYWLCMLGTLVGLSATLAPNSCPDKHYWTGGGLCCRMCEPGTFFVKDCEQDRTAAQCDPCIPGTSFSPDYHTRPHCESCRHCNSGFLIRNCTVTANAECSCSKNWQCRDQECTECDPPLNPALTRQPSETPSPQPPPTHLPHGTEKPSWPLHRQLPNSTVYSQRSSHRPLCSSDCIRIFVTFSSMFLIFVLGAILFFHQRRNHGPNEDRQAVPEEPCPYSCPREEEGSAIPIQEDYRKPEPAFYP

SEQ ID NO: 60 - DNA, 660 bp, Homo sapiens
  source 1..660: mol_type = other DNA
atgctgcgcctgctgctggcgctgaacctgtttccgagcattcaggtgaccggcaacaaaattctggtgaaacagagcccgatgctggtggcgtatgataacgcggtgaacctgagctgcaaatatagctataacctgtttagccgcgaatttcgcgcgagcctgcataaaggcctggatagcgcggtggaagtgtgcgtggtgtatggcaactatagccagcagctgcaggtgtatagcaaaaccggctttaactgcgatggcaaactgggcaacgaaagcgtgaccttttatctgcagaacctgtatgtgaaccagaccgatatttatttttgcaaaattgaagtgatgtatccgccgccgtatctggataacgaaaaaagcaacggcaccattattcatgtgaaaggcaaacatctgtgcccgagcccgctgtttccgggcccgagcaaaccgttttgggtgctggtggtggtgggcggcgtgctggcgtgctatagcctgctggtgaccgtggcgtttattattttttgggtgcgcagcaaacgcagccgcctgctgcatagcgattatatgaacatgaccccgcgccgcccgggcccgacccgcaaacattatcagccgtatgcgccgccgcgcgattttgcggcgtatcgcagc

SEQ ID NO: 61 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 62 - DNA, 654 bp, Mus musculus
  source 1..654: mol_type = other DNA
atgaccctgcgcctgctgtttctggcgctgaacttttttagcgtgcaggtgaccgaaaacaaaattctggtgaaacagagcccgctgctggtggtggatagcaacgaagtgagcctgagctgccgctatagctataacctgctggcgaaagaatttcgcgcgagcctgtataaaggcgtgaacagcgatgtggaagtgtgcgtgggcaacggcaactttacctatcagccgcagtttcgcagcaacgcggaatttaactgcgatggcgattttgataacgaaaccgtgacctttcgcctgtggaacctgcatgtgaaccataccgatatttatttttgcaaaattgaatttatgtatccgccgccgtatctggataacgaacgcagcaacggcaccattattcatattaaagaaaaacatctgtgccatacccagagcagcccgaaactgttttgggcgctggtggtggtggcgggcgtgctgttttgctatggcctgctggtgaccgtggcgctgtgcgtgatttggaccaacagccgccgcaaccgcctgctgcagagcgattatatgaacatgaccccgcgccgcccgggcctgacccgcaaaccgtatcagccgtatgcgccggcgcgcgattttgcggcgtatcgcccg

SEQ ID NO: 63 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 64 - DNA, 768 bp, Homo sapiens
  source 1..768: mol_type = other DNA
atgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtga

SEQ ID NO: 65 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 66 - DNA, 768 bp, Mus musculus
  source 1..768: mol_type = other DNA
atgggcaacaactgctataacgtggtggtgattgtgctgctgctggtgggctgcgaaaaagtgggcgcggtgcagaacagctgcgataactgccagccgggcaccttttgccgcaaatataacccggtgtgcaaaagctgcccgccgagcacctttagcagcattggcggccagccgaactgcaacatttgccgcgtgtgcgcgggctattttcgctttaaaaaattttgcagcagcacccataacgcggaatgcgaatgcattgaaggctttcattgcctgggcccgcagtgcacccgctgcgaaaaagattgccgcccgggccaggaactgaccaaacagggctgcaaaacctgcagcctgggcacctttaacgatcagaacggcaccggcgtgtgccgcccgtggaccaactgcagcctggatggccgcagcgtgctgaaaaccggcaccaccgaaaaagatgtggtgtgcggcccgccggtggtgagctttagcccgagcaccaccattagcgtgaccccggaaggcggcccgggcggccatagcctgcaggtgctgaccctgtttctggcgctgaccagcgcgctgctgctggcgctgatttttattaccctgctgtttagcgtgctgaaatggattcgcaaaaaatttccgcatatttttaaacagccgtttaaaaaaaccaccggcgcggcgcaggaagaagatgcgtgcagctgccgctgcccgcaggaagaagaaggcggcggcggcggctatgaactg

SEQ ID NO: 67 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 68 - DNA, 831 bp, Homo sapiens
  source 1..831: mol_type = other DNA
atgtgcgtgggcgcgcgccgcctgggccgcggcccgtgcgcggcgctgctgctgctgggcctgggcctgagcaccgtgaccggcctgcattgcgtgggcgatacctatccgagcaacgatcgctgctgccatgaatgccgcccgggcaacggcatggtgagccgctgcagccgcagccagaacaccgtgtgccgcccgtgcggcccgggcttttataacgatgtggtgagcagcaaaccgtgcaaaccgtgcacctggtgcaacctgcgcagcggcagcgaacgcaaacagctgtgcaccgcgacccaggataccgtgtgccgctgccgcgcgggcacccagccgctggatagctataaaccgggcgtggattgcgcgccgtgcccgccgggccattttagcccgggcgataaccaggcgtgcaaaccgtggaccaactgcaccctggcgggcaaacataccctgcagccggcgagcaacagcagcgatgcgatttgcgaagatcgcgatccgccggcgacccagccgcaggaaacccagggcccgccggcgcgcccgattaccgtgcagccgaccgaagcgtggccgcgcaccagccagggcccgagcacccgcccggtggaagtgccgggcggccgcgcggtggcggcgattctgggcctgggcctggtgctgggcctgctgggcccgctggcgattctgctggcgctgtatctgctgcgccgcgatcagcgcctgccgccggatgcgcataaaccgccgggcggcggcagctttcgcaccccgattcaggaagaacaggcggatgcgcatagcaccctggcgaaaatt

SEQ ID NO: 69 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 70 - DNA, 816 bp, Mus musculus
  source 1..816: mol_type = other DNA
atgtatgtgtgggtgcagcagccgaccgcgctgctgctgctggcgctgaccctgggcgtgaccgcgcgccgcctgaactgcgtgaaacatacctatccgagcggccataaatgctgccgcgaatgccagccgggccatggcatggtgagccgctgcgatcatacccgcgataccctgtgccatccgtgcgaaaccggcttttataacgaagcggtgaactatgatacctgcaaacagtgcacccagtgcaaccatcgcagcggcagcgaactgaaacagaactgcaccccgacccaggataccgtgtgccgctgccgcccgggcacccagccgcgccaggatagcggctataaactgggcgtggattgcgtgccgtgcccgccgggccattttagcccgggcaacaaccaggcgtgcaaaccgtggaccaactgcaccctgagcggcaaacagacccgccatccggcgagcgatagcctggatgcggtgtgcgaagatcgcagcctgctggcgaccctgctgtgggaaacccagcgcccgacctttcgcccgaccaccgtgcagagcaccaccgtgtggccgcgcaccagcgaactgccgagcccgccgaccctggtgaccccggaaggcccggcgtttgcggtgctgctgggcctgggcctgggcctgctggcgccgctgaccgtgctgctggcgctgtatctgctgcgcaaagcgtggcgcctgccgaacaccccgaaaccgtgctggggcaacagctttcgcaccccgattcaggaagaacataccgatgcgcattttaccctggcgaaaatt

SEQ ID NO: 71 - protein, 272 aa, Mus musculus
MYVWVQQPTALLLLALTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 72 - DNA, 597 bp, Homo sapiens
  source 1..597: mol_type = other DNA
atgaaaagcggcctgtggtatttttttctgttttgcctgcgcattaaagtgctgaccggcgaaattaacggcagcgcgaactatgaaatgtttatttttcataacggcggcgtgcagattctgtgcaaatatccggatattgtgcagcagtttaaaatgcagctgctgaaaggcggccagattctgtgcgatctgaccaaaaccaaaggcagcggcaacaccgtgagcattaaaagcctgaaattttgccatagccagctgagcaacaacagcgtgagcttttttctgtataacctggatcatagccatgcgaactattatttttgcaacctgagcatttttgatccgccgccgtttaaagtgaccctgaccggcggctatctgcatatttatgaaagccagctgtgctgccagctgaaattttggctgccgattggctgcgcggcgtttgtggtggtgtgcattctgggctgcattctgatttgctggctgaccaaaaaaaaatatagcagcagcgtgcatgatccgaacggcgaatatatgtttatgcgcgcggtgaacaccgcgaaaaaaagccgcctgaccgatgtgaccctg

SEQ ID NO: 73 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 74 - DNA, 600 bp, Mus musculus
  source 1..600: mol_type = other DNA
atgaaaccgtatttttgccgcgtgtttgtgttttgctttctgattcgcctgctgaccggcgaaattaacggcagcgcggatcatcgcatgtttagctttcataacggcggcgtgcagattagctgcaaatatccggaaaccgtgcagcagctgaaaatgcgcctgtttcgcgaacgcgaagtgctgtgcgaactgaccaaaaccaaaggcagcggcaacgcggtgagcattaaaaacccgatgctgtgcctgtatcatctgagcaacaacagcgtgagcttttttctgaacaacccggatagcagccagggcagctattatttttgcagcctgagcatttttgatccgccgccgtttcaggaacgcaacctgagcggcggctatctgcatatttatgaaagccagctgtgctgccagctgaaactgtggctgccggtgggctgcgcggcgtttgtggtggtgctgctgtttggctgcattctgattatttggtttagcaaaaaaaaatatggcagcagcgtgcatgatccgaacagcgaatatatgtttatggcggcggtgaacaccaacaaaaaaagccgcctggcgggcgtgaccagc

SEQ ID NO: 75 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 76 - DNA, 279 bp, Homo sapiens
  source 1..279: mol_type = other DNA
atgattcatctgggccatattctgtttctgctgctgctgccggtggcggcggcgcagaccaccccgggcgaacgcagcagcctgccggcgttttatccgggcaccagcggcagctgcagcggctgcggcagcctgagcctgccgctgctggcgggcctggtggcggcggatgcggtggcgagcctgctgattgtgggcgcggtgtttctgtgcgcgcgcccgcgccgcagcccggcgcaggaagatggcaaagtgtatattaacatgccgggccgcggc

SEQ ID NO: 77 - protein, 93 aa, Homo sapiens
MIHLGHILFLLLLPVAAAQTTPGERSSLPAFYPGTSGSCSGCGSLSLPLLAGLVAADAVASLLIVGAVFLCARPRRSPAQEDGKVYINMPGRG

SEQ ID NO: 78 - DNA, 237 bp, Mus musculus
  source 1..237: mol_type = other DNA
atggatccgccgggctatctgctgtttctgctgctgctgccggtggcggcgagccagaccagcgcgggcagctgcagcggctgcggcaccctgagcctgccgctgctggcgggcctggtggcggcggatgcggtgatgagcctgctgattgtgggcgtggtgtttgtgtgcatgcgcccgcatggccgcccggcgcaggaagatggccgcgtgtatattaacatgccgggccgcggc

SEQ ID NO: 79 - protein, 79 aa, Mus musculus
MDPPGYLLFLLLLPVAASQTSAGSCSGCGTLSLPLLAGLVAADAVMSLLIVGVVFVCMRPHGRPAQEDGRVYINMPGRG

SEQ ID NO: 80 - DNA, 342 bp, Homo sapiens
  source 1..342: mol_type = other DNA
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatga

SEQ ID NO: 81 - protein, 113 aa, Homo sapiens
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 82 - DNA, 345 bp, Mus musculus
  source 1..345: mol_type = other DNA
atgggggctctggagccctcctggtgccttctgttccttcctgtcctcctgactgtgggaggattaagtcccgtacaggcccagagtgacactttcccaagatgcgactgttcttccgtgagccctggtgtactggctgggattgttctgggtgacttggtgttgactctgctgattgccctggctgtgtactctctgggccgcctggtctcccgaggtcaagggacagcggaagggacccggaaacaacacattgctgagactgagtcgccttatcaggagcttcagggtcagagaccagaagtatacagtgacctcaacacacagaggcaatattacagatga

SEQ ID NO: 83 - protein, 114 aa, Mus musculus
MGALEPSWCLLFLPVLLTVGGLSPVQAQSDTFPRCDCSSVSPGVLAGIVLGDLVLTLLIALAVYSLGRLVSRGQGTAEGTRKQHIAETESPYQELQGQRPEVYSDLNTQRQYYR

SEQ ID NO: 84 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 85 - DNA, 492 bp, Homo sapiens
  source 1..492: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 86 - protein, 164 aa, Mus musculus
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 87 - DNA, 495 bp, Mus musculus
  source 1..495: mol_type = other DNA
atgaagtggaaagtgtctgttctcgcctgcatcctccacgtgcggttcccaggagcagaggcacagagctttggtctgctggatcccaaactctgctacttgctagatggaatcctcttcatctacggagtcatcatcacagccctgtacctgagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgctaa

SEQ ID NO: 88 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 89 - DNA, 762 bp, Homo sapiens
  source 1..762: mol_type = other DNA
atgtggcagctgctgctgccgaccgcgctgctgctgctggtgagcgcgggcatgcgcaccgaagatctgccgaaagcggtggtgtttctggaaccgcagtggtatcgcgtgctggaaaaagatagcgtgaccctgaaatgccagggcgcgtatagcccggaagataacagcacccagtggtttcataacgaaagcctgattagcagccaggcgagcagctattttattgatgcggcgaccgtggatgatagcggcgaatatcgctgccagaccaacctgagcaccctgagcgatccggtgcagctggaagtgcatattggctggctgctgctgcaggcgccgcgctgggtgtttaaagaagaagatccgattcatctgcgctgccatagctggaaaaacaccgcgctgcataaagtgacctatctgcagaacggcaaaggccgcaaatattttcatcataacagcgatttttatattccgaaagcgaccctgaaagatagcggcagctatttttgccgcggcctgtttggcagcaaaaacgtgagcagcgaaaccgtgaacattaccattacccagggcctggcggtgagcaccattagcagcttttttccgccgggctatcaggtgagcttttgcctggtgatggtgctgctgtttgcggtggataccggcctgtattttagcgtgaaaaccaacattcgcagcagcacccgcgattggaaagatcataaatttaaatggcgcaaagatccgcaggataaa

SEQ ID NO: 90 - protein, 261 aa, Mus musculus
MFQNAHSGSQWLLPPLTILLLFAFADRQSAALPKAVVKLDPPWIQVLKEDMVTLMCEGTHNPGNSSTQWFHNGRSIRSQVQASYTFKATVNDSGEYRCQMEQTRLSDPVDLGVISDWLLLQTPQRVFLEGETITLRCHSWRNKLLNRISFFHNEKSVRYHHYKSNFSIPKANHSHSGDYYCKGSLGSTQHQSKPVTITVQDPATTSSISLVWYHTAFSLVMCLLFAVDTGLYFYVRRNLQTPREYWRKSLSIRKHQAPQDK

SEQ ID NO: 91 - DNA, 786 bp, Mus musculus
  source 1..786: mol_type = other DNA
atgtttcagaatgcacactctggaagccaatggctacttccaccactgacaattctgctgctgtttgcttttgcagacaggcagagtgcagctcttccgaaggctgtggtgaaactggaccccccatggatccaggtgctcaaggaagacatggtgacactgatgtgcgaagggacccacaaccctgggaactcttctacccagtggttccacaacgggaggtccatccggagccaggtccaagccagttacacgtttaaggccacagtcaatgacagtggagaatatcggtgtcaaatggagcagacccgcctcagcgaccctgtagatctgggagtgatttctgactggctgctgctccagacccctcagcgggtgtttctggaaggggaaaccatcacgctaaggtgccatagctggaggaacaaactactgaacaggatctcattcttccataatgaaaaatccgtgaggtatcatcactacaaaagtaatttctctatcccaaaagccaaccacagtcacagtggggactactactgcaaaggaagtctaggaagtacacagcaccagtccaagcctgtcaccatcactgtccaagatccagcaactacatcctccatctctctagtctggtaccacactgctttctccctagtgatgtgcctcctgtttgcagtggacacgggcctttatttctacgtacggagaaatcttcaaaccccgagggagtactggaggaagtccctgtcaatcagaaagcaccaggctcctcaagacaagtga

SEQ ID NO: 92 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 93 - DNA, 648 bp, Homo sapiens
  source 1..648: mol_type = other DNA
atgggctggattcgcggccgccgcagccgccatagctgggaaatgagcgaatttcataactataacctggatctgaaaaaaagcgattttagcacccgctggcagaaacagcgctgcccggtggtgaaaagcaaatgccgcgaaaacgcgagcccgttttttttttgctgctttattgcggtggcgatgggcattcgctttattattatggtggcgatttggagcgcggtgtttctgaacagcctgtttaaccaggaagtgcagattccgctgaccgaaagctattgcggcccgtgcccgaaaaactggatttgctataaaaacaactgctatcagttttttgatgaaagcaaaaactggtatgaaagccaggcgagctgcatgagccagaacgcgagcctgctgaaagtgtatagcaaagaagatcaggatctgctgaaactggtgaaaagctatcattggatgggcctggtgcatattccgaccaacggcagctggcagtgggaagatggcagcattctgagcccgaacctgctgaccattattgaaatgcagaaaggcgattgcgcgctgtatgcgagcagctttaaaggctatattgaaaactgcagcaccccgaacacctatatttgcatgcagcgcaccgtg

SEQ ID NO: 94 - protein, 232 aa, Mus musculus
MALIRDRKSHHSEMSKCHNYDLKPAKWDTSQEQQKQRLALTTSQPGENGIIRGRYPIEKLKISPMFVVRVLAIALAIRFTLNTLMWLAIFKETFQPVLCNKEVPVSSREGYCGPCPNNWICHRNNCYQFFNEEKTWNQSQASCLSQNSSLLKIYSKEEQDFLKLVKSYHWMGLVQIPANGSWQWEDGSSLSYNQLTLVEIPKGSCAVYGSSFKAYTEDCANLNTYICMKRAV

SEQ ID NO: 95 - DNA, 696 bp, Mus musculus
  source 1..696: mol_type = other DNA
atggcgctgattcgcgatcgcaaaagccatcatagcgaaatgagcaaatgccataactatgatctgaaaccggcgaaatgggataccagccaggaacagcagaaacagcgcctggcgctgaccaccagccagccgggcgaaaacggcattattcgcggccgctatccgattgaaaaactgaaaattagcccgatgtttgtggtgcgcgtgctggcgattgcgctggcgattcgctttaccctgaacaccctgatgtggctggcgatttttaaagaaacctttcagccggtgctgtgcaacaaagaagtgccggtgagcagccgcgaaggctattgcggcccgtgcccgaacaactggatttgccatcgcaacaactgctatcagttttttaacgaagaaaaaacctggaaccagagccaggcgagctgcctgagccagaacagcagcctgctgaaaatttatagcaaagaagaacaggattttctgaaactggtgaaaagctatcattggatgggcctggtgcagattccggcgaacggcagctggcagtgggaagatggcagcagcctgagctataaccagctgaccctggtggaaattccgaaaggcagctgcgcggtgtatggcagcagctttaaagcgtataccgaagattgcgcgaacctgaacacctatatttgcatgaaacgcgcggtg

SEQ ID NO: 96 - protein, 4 aa, synthetic construct
YMNM

SEQ ID NO: 97 - protein, 4 aa, synthetic construct
PYAP

SEQ ID NO: 98 - protein, 4 aa, synthetic construct
FMNM

SEQ ID NO: 99 - protein, 4 aa, synthetic construct
AYAA

SEQ ID NO: 100 - protein, 21 aa, synthetic construct
ATMGWSCIILFLVATATGVHS

SEQ ID NO: 101 - DNA, 57 bp, synthetic construct
  source 1..57: mol_type = other DNA
atgggatggagctgtatcatcctcttcttggtagcaacagctaccggtgtgcactcc

SEQ ID NO: 102 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 103 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 445 aa, synthetic construct
EVQLLESGGGLVQPGGSLRLSCAASGFTFSSYAMSWVRQAPGKGLEWVSAIIGSGASTYYADSVKGRFTISRDNSKNTLYLQMNSLRAEDTAVYYCAKGWFGGFNYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 105 - protein, 215 aa, synthetic construct
EIVLTQSPGTLSLSPGERATLSCRASQSVTSSYLAWYQQKPGQAPRLLINVGSRRATGIPDRFSGSGSGTDFTLTISRLEPEDFAVYYCQQGIMLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 449 aa, synthetic construct
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 107 - protein, 223 aa, synthetic construct
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 449 aa, synthetic construct
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 109 - protein, 218 aa, synthetic construct
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 449 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 111 - protein, 215 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 447 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 113 - protein, 213 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 449 aa, synthetic construct
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARLYGYAYYGAFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 115 - protein, 214 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQNGLQPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116-118 - skipped (3 entries)

SEQ ID NO: 119 - protein, 4 aa, synthetic construct
GSGN

SEQ ID NO: 120 - protein, 328 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 121 - protein, 276 aa, Homo sapiens
MRNQAPGRPKGATFPPRRPTGSRAPPLAPELRAKQRPGERVMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 122 - DNA, 828 bp, Homo sapiens
  source 1..828: mol_type = other DNA
atgcgcaaccaggcgccgggccgcccgaaaggcgcgacctttccgccgcgccgcccgaccggcagccgcgcgccgccgctggcgccggaactgcgcgcgaaacagcgcccgggcgaacgcgtgatggcgctgccggtgaccgcgctgctgctgccgctggcgctgctgctgcatgcggcgcgcccgagccagtttcgcgtgagcccgctggatcgcacctggaacctgggcgaaaccgtggaactgaaatgccaggtgctgctgagcaacccgaccagcggctgcagctggctgtttcagccgcgcggcgcggcggcgagcccgacctttctgctgtatctgagccagaacaaaccgaaagcggcggaaggcctggatacccagcgctttagcggcaaacgcctgggcgatacctttgtgctgaccctgagcgattttcgccgcgaaaacgaaggctattatttttgcagcgcgctgagcaacagcattatgtattttagccattttgtgccggtgtttctgccggcgaaaccgaccaccaccccggcgccgcgcccgccgaccccggcgccgaccattgcgagccagccgctgagcctgcgcccggaagcgtgccgcccggcggcgggcggcgcggtgcatacccgcggcctggattttgcgtgcgatatttatatttgggcgccgctggcgggcacctgcggcgtgctgctgctgagcctggtgattaccctgtattgcaaccatcgcaaccgccgccgcgtgtgcaaatgcccgcgcccggtggtgaaaagcggcgataaaccgagcctgagcgcgcgctatgtg

SEQ ID NO: 123 - protein, 247 aa, Homo sapiens
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 124 - DNA, 741 bp, Homo sapiens
  source 1..741: mol_type = other DNA
atggcgagcccgctgacccgctttctgagcctgaacctgctgctgatgggcgaaagcattattctgggcagcggcgaagcgaaaccgcaggcgccggaactgcgcatttttccgaaaaaaatggatgcggaactgggccagaaagtggatctggtgtgcgaagtgctgggcagcgtgagccagggctgcagctggctgtttcagaacagcagcagcaaactgccgcagccgacctttgtggtgtatatggcgagcagccataacaaaattacctgggatgaaaaactgaacagcagcaaactgtttagcgcggtgcgcgataccaacaacaaatatgtgctgaccctgaacaaatttagcaaagaaaacgaaggctattatttttgcagcgtgattagcaacagcgtgatgtattttagcagcgtggtgccggtgctgcagaaagtgaacagcaccaccaccaaaccggtgctgcgcaccccgagcccggtgcatccgaccggcaccagccagccgcagcgcccggaagattgccgcccgcgcggcagcgtgaaaggcaccggcctggattttgcgtgcgatatttatatttgggcgccgctggcgggcatttgcgtggcgccgctgctgagcctgattattaccctgatttgctatcatcgcagccgcaaacgcgtgtgcaaatgcccgcgcccgctggtgcgccaggaaggcaaaccgcgcccgagcgaaaaaattgtg

SEQ ID NO: 125 - protein, 476 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSLKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 248 aa, synthetic construct
EVQLVESGGGLVQPGGSLRLSCAASGFDFSRYWMNWVRQAPGKGLEWVGEITPDSSTINYTPSLKGRFTISRDNAKNSLYLQMNSLRAEDTAVYYCVRPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 127 - protein, 476 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVLGGGGSLKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 248 aa, synthetic construct
EVQLVESGGGLVQPGGSLRLSCAASGFDFSRYWMNWVRQAPGKGLEWVGEITPDSSTINYAPSLKGRFTISRDNAKNSLYLQMNSLRAEDTAVYYCVRPYDYGAWFASWGQGTLVTVSSGGGGSGGGGSGGGGSGGGGSQAVVTQEPSLTVSPGGTVTLTCRSSTGAVTTSNYANWVQEKPDHLFTGLIGGTNKRAPGTPARFSGSLLGGKAALTLSGAQPEDEAEYYCALWYSNHWVFGGGTKLTVL

SEQ ID NO: 129 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 130 - protein, 360 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDEL

SEQ ID NO: 131 - protein, 219 aa, synthetic construct; identical to SEQ ID NO: 103

SEQ ID NO: 132 - protein, 447 aa, synthetic construct; identical to SEQ ID NO: 102

SEQ ID NO: 133 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

(19) United States
(12) Patent Application Publication
(10) Pub. No.: US 2024/0269176 A1
(43) Pub. Date: Aug. 15, 2024

(54) HETERODIMERIC FC DOMAIN ANTIBODIES

(71) Applicant: Hoffmann-La Roche Inc., Little Falls, NJ (US)

(72) Inventors: Diana DAROWSKI, Basel (CH); Anne FREIMOSER-GRUNDSCHOBER, Zurich (CH); Christian KLEIN, Bonstetten (CH); Ekkehard MOESSNER, Kreuzlingen (CH)

(73) Assignee: Hoffmann-La Roche Inc., Little Falls, NJ (US)

(21) Appl. No.: 18/417,644

(22) Filed: Jan. 19, 2024

Related U.S. Application Data
(63) Continuation of application No. PCT/EP2022/070338, filed on Jul. 20, 2022.

(30) Foreign Application Priority Data
Jul. 22, 2021 (EP) 21187191.8

(51) Int. Cl. A61K 35/17; A61K 39/00; A61P 35/00; C07K 16/28

(52) U.S. Cl. CPC A61K 35/17; A61K 39/4611; A61K 39/4631; A61K 39/464411; A61P 35/00; C07K 16/283; C07K 16/2887; A61K 2039/505; A61K 2039/5156; A61K 2239/13; A61K 2239/21; C07K 2317/24; C07K 2317/31; C07K 2317/41; C07K 2317/52; C07K 2317/732

(57) ABSTRACT

The present invention generally relates to heterodimeric Fc domain antibodies as well as to combination with antigen binding receptors capable of specific binding to such antibodies comprising the amino acid mutation P329G according to EU numbering. The present invention also relates to T cells, transduced with such antigen binding receptor and kits comprising the transduced T cells and tumor targeting antibodies comprising such heterodimeric Fc domains.

### CROSS-REFERENCE TO RELATED APPLICATIONS

This application is a continuation of International Application No. PCT/EP2022/070338 having an international filing date of Jul. 20, 2022, which claims benefit of priority to European Patent Application No. 21187191.8, filed Jul. 22, 2021, each of which is incorporated herein by reference in its entirety.

### SEQUENCE LISTING

This application contains a Sequence Listing which has been submitted electronically in XML format and is hereby incorporated by reference in its entirety. Said XML copy, created on Jan. 19, 2024, is named P36828-US_Seq_List.xml and is 156,801 bytes in size.

### FIELD OF THE INVENTION

The present invention generally relates to heterodimeric Fc domain antibodies as well as to combination with antigen binding receptors capable of specific binding to such antibodies comprising the amino acid mutation P329G according to EU numbering. The present invention also relates to T cells, transduced with such antigen binding receptor and kits comprising the transduced T cells and tumor targeting antibodies comprising such heterodimeric Fc domains.

### BACKGROUND

Adoptive T cell therapy (ACT) is a powerful treatment approach using cancer-specific T cells (Rosenberg and Restifo, Science 348(6230) (2015), 62-68). ACT may use naturally occurring tumor-specific cells or T cells rendered specific by genetic engineering using T cell or chimeric antigen receptors (Rosenberg and Restifo, Science 348(6230) (2015), 62-68). ACT can successfully treat and induce remission in patients suffering even from advanced and otherwise treatment refractory diseases such as acute lymphatic leukemia, non-hodgkins lymphoma or melanoma (Dudley et al., J Clin Oncol 26(32) (2008), 5233-5239; Grupp et al., N Engl J Med 368 (16) (2013), 1509-1518; Kochenderfer et al., J Clin Oncol. (2015) 33(6):540-549, doi: 10.1200/JCO.2014.56.2025. Epub 2014 Aug. 25).

However, despite impressive clinical efficacy, ACT is limited by treatment-related toxicities. The specificity, and resulting on-target and off-target effects, of engineered T cells used in ACT is mainly driven by the tumor targeting antigen binding moiety implemented in the antigen binding receptors. Non-exclusive expression of the tumor antigen or temporal difference in the expression level can result with serious side effects or even abortion of ACT due to non-tolerable toxicity of the treatment.

Additionally, the availability of tumor-specific T cells for efficient tumor cells lysis is dependent on the long-term survival and proliferation capacity of engineered T cells in vivo. On the other hand, in vivo survival and proliferation of T cells may also result in unwanted long-term effects due to the persistence of an uncontrolled T cell response which can result in damage of healthy tissue (Grupp et al. 2013 N Engl J Med 368(16):1509-18, Maude et al. 2014 2014 N Engl J Med 371(16):1507-17).

One approach for limiting serious treatment-related toxicities and to improve safety of ACT is to restrict the activation and proliferation of T cells by introducing adaptor molecules in the immunological synapse. Such adaptor molecules comprise small molecular bimodular switches as e.g. recently described folate-FITC switch (Kim et al. J Am Chem Soc 2015; 137:2832-2835). A further approach included artificially modified antibodies comprising a tag to guide and direct the specificity of the T cells to target tumor cells (Ma et al. PNAS 2016; 113(4):E450-458, Cao et al. Angew Chem 2016; 128:1-6, Rogers et al. PNAS 2016; 113(4):E459-468, Tamada et al. Clin Cancer Res 2012; 18(23):6436-6445).

However, existing approaches have several limitations. Immunological synapses relying on molecular switches require introduction of additional elements that might elicit an immune response or result with non-specific off-target effects. Furthermore, the complexity of such multicomponent systems may limit treatment efficacy and tolerability. On the other hand, the introduction of tag structure in existing therapeutic monoclonal antibodies may affect the efficacy and safety profile of these constructs. Further, adding tags require additional modification and purification steps making the production of such antibodies more complex and further require additional safety testing.

Furthermore, antigen binding receptors capable of specific binding to mutated domains with reduced Fc receptor binding have been described earlier by the present inventors (WO2018/177966).

There is still a need for improved adoptive T cell therapies having the potential to improve safety and/or efficacy in the treatment of cancer patients.

### SUMMARY OF THE INVENTION

Herein provided are antibodies comprising a heterodimeric Fc domain composed of a first and a second subunit, wherein the first subunit comprises the amino acid mutation P329G according to EU numbering and wherein the second subunit comprises a proline (P) at position 329 according to EU numbering. The antibodies according to the present invention are able to efficiently recruit anti-P329G CAR-T cells for killing. Furthermore, the antibodies according to the present invention are able to efficiently recruit innate immune cells such as NK cells or monocytes for FcgR dependent ADCC without unspecific cross-activation.

Recruiting innate immune cells at the same time with CAR-T cells may inter alia help to reduce adverse events (e.g. cytokine release syndrome) by giving first the antibody and infusing the CAR-T cells only at a later time point when the antibody has already induced ADCC-mediated anti-tumor efficacy and debulking. Furthermore, recruiting innate immune cells at the same time with CAR-T cells may inter alia help in generating a secondary immune response by activating antigen presenting cells such as FcgR expressing monocytes, macrophages and dendritic cells in the tumor microenvironment.

Accordingly, provided is an antibody comprising a heterodimeric Fc domain composed of a first and a second subunit, wherein the first subunit comprises the amino acid mutation P329G according to EU numbering and wherein the second subunit comprises a proline (P) at position 329 according to EU numbering.

In one aspect, the Fc domain is an IgG, particularly an IgG₁, Fc domain.

In one aspect, the Fc domain is a human Fc domain.

In one aspect, the Fc domain comprises a modification promoting the association of the first and the second subunit of the Fc domain.

In one aspect, the antibody is defucosylated.

In one aspect, the heterodimeric Fc domain exhibits increased binding affinity to an Fc receptor and/or increased effector function, as compared to a native IgG₁ Fc domain, in particular wherein the effector function is ADCC.

In one aspect, the heterodimeric Fc domain comprises one or more amino acid mutations that increase binding to an Fc receptor and/or effector function, in particular wherein the effector function is ADCC.

In one aspect, the antibody comprises at least one antigen binding moiety capable of specific binding to an antigen on a target cell.

In one aspect, the target cell is a cancer cell.

In one aspect, the antigen is selected from the group consisting of FAP, CEA, p95 HER2, BCMA, EpCAM, MSLN, MCSP, HER-1, HER-2, HER-3, CD19, CD20, CD22, CD33, CD38, CD52Flt3, EpCAM, IGF-1R, FOLR1, Trop-2, CA-12-5, HLA-DR, MUC-1 (mucin), GD2, A33-antigen, PSMA, PSCA, transferrin-receptor, TNC (tenascin) and CA-IX.

In one aspect, the antigen binding moiety is a scFv, a Fab, a crossFab or a scFab.

In one aspect, the antibody is a human, humanized or chimeric antibody.

In one aspect, the antibody is a multispecific antibody.

Further provided is an isolated polynucleotide encoding the antibody as herein described.

Further provided is a host cell comprising the isolated polynucleotide as herein described.

Further provided is a method of producing an antibody, comprising the steps of (a) culturing the host cell as herein described under conditions suitable for the expression of the antibody and optionally (b) recovering the antibody.

Further provided is an antibody produced by the method as herein described.

Further provided is a pharmaceutical composition comprising the antibody as herein described and a pharmaceutically acceptable carrier.

Further provided is the antibody as herein described and a transduced T cell for use in combination in the treatment of cancer, wherein the transduced T cell expresses an antigen binding receptor capable of specific binding to the first subunit.

In one aspect, the antigen binding receptor is capable of specific binding to an Fc domain subunit comprising the amino acid mutation P329G according to EU numbering.

In one aspect, the antigen binding receptor comprises a heavy chain variable domain (VH) comprising:
(a) a heavy chain complementarity determining region (CDR H) 1 amino acid sequence of RYWMN (SEQ ID NO:1);
(b) a CDR H2 amino acid sequence of EITPDSSTINYAPSLKG (SEQ ID NO:2) or of EITPDSSTINYTPSLKG (SEQ ID NO:40);
(c) a CDR H3 amino acid sequence of PYDYGAWFAS (SEQ ID NO:3); and a light chain variable domain (VL) comprising:
(d) a light chain (CDR L)1 amino acid sequence of RSSTGAVTTSNYAN (SEQ ID NO:4);
(e) a CDR L2 amino acid sequence of GTNKRAP (SEQ ID NO:5); and
(f) a CDR L3 amino acid sequence of ALWYSNHWV (SEQ ID NO:6).

In one aspect, the antigen binding receptor comprises
(i) a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof, in particular the CD28 transmembrane domain or a fragment thereof,
(ii) at least one stimulatory signaling domain selected from the group consisting of the intracellular domain of CD3z, of FCGR3A and of NKG2D, or fragments thereof, in particular wherein the at least one stimulatory signaling domain is the CD3z intracellular domain or a fragment thereof, and/or
(iii) at least one co-stimulatory signaling domain individually selected from the group consisting of the intracellular domain of CD27, of CD28, of CD137, of OX40, of ICOS, of DAP10 and of DAP12, or fragments thereof, in particular wherein the at least one co-stimulatory signaling domain is the CD28 intracellular domain or a fragment thereof.

In one aspect, the transduced T cell is administered before, simultaneously with or after administration of the antibody.

Further provided is a method of treating or delaying progression of a cancer in an individual comprising administering to said individual an effective amount of an antibody and a transduced T cell, wherein the antibody comprises a heterodimeric Fc domain composed of a first and a second subunit, wherein the first subunit comprises the amino acid mutation P329G according to EU numbering, wherein the second subunit comprises a proline (P) at position 329 according to EU numbering, and wherein the transduced T cell expresses an antigen binding receptor capable of specific binding to the first subunit.

In one aspect of the method, the antigen binding receptor is capable of specific binding to an Fc domain subunit comprising the amino acid mutation P329G according to EU numbering.

In one aspect of the method, the antigen binding receptor comprises a heavy chain variable domain (VH) comprising:
(a) a heavy chain complementarity determining region (CDR H) 1 amino acid sequence of RYWMN (SEQ ID NO:1);
(b) a CDR H2 amino acid sequence of EITPDSSTINYAPSLKG (SEQ ID NO:2) or of EITPDSSTINYTPSLKG (SEQ ID NO:40);
(c) a CDR H3 amino acid sequence of PYDYGAWFAS (SEQ ID NO:3); and a light chain variable domain (VL) comprising:
(d) a light chain (CDR L)1 amino acid sequence of RSSTGAVTTSNYAN (SEQ ID NO:4);
(e) a CDR L2 amino acid sequence of GTNKRAP (SEQ ID NO:5); and
(f) a CDR L3 amino acid sequence of ALWYSNHWV (SEQ ID NO:6).

In one aspect of the method, the antigen binding receptor comprises:
(i) a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof, in particular the CD28 transmembrane domain or a fragment thereof,
(ii) at least one stimulatory signaling domain selected from the group consisting of the intracellular domain of CD3z, of FCGR3A and of NKG2D, or fragments thereof, in particular wherein the at least one stimulatory signaling domain is the CD3z intracellular domain or a fragment thereof, and/or
(iii) at least one co-stimulatory signaling domain individually selected from the group consisting of the intracellular domain of CD27, of CD28, of CD137, of OX40, of ICOS, of DAP10 and of DAP12, or fragments thereof, in particular wherein the at least one co-stimulatory signaling domain is the CD28 intracellular domain or a fragment thereof.

In one aspect, the transduced T cell is administered before, simultaneously with or after administration of the antibody.

Further provided is the use of an antibody in the manufacture of a medicament for use in combination with a transduced T cell in the treatment of cancer, wherein the antibody comprises a hetereomeric Fc domain composed of a first and a second subunit, wherein the first subunit comprises the amino acid mutation P329G according to EU numbering, wherein the second subunit comprises a proline (P) at position 329 according to EU numbering, and wherein the transduced T cell expresses an antigen binding receptor capable of specific binding to the first subunit.

In one aspect of the use, the antigen binding receptor is capable of specific binding to an Fc domain subunit comprising the amino acid mutation P329G according to EU numbering.

In one aspect, the antigen binding receptor comprises a heavy chain variable domain (VH) comprising:
(a) a heavy chain complementarity determining region (CDR H) 1 amino acid sequence of RYWMN (SEQ ID NO:1);
(b) a CDR H2 amino acid sequence of EITPDSSTINYAPSLKG (SEQ ID NO:2) or of EITPDSSTINYTPSLKG (SEQ ID NO:40);
(c) a CDR H3 amino acid sequence of PYDYGAWFAS (SEQ ID NO:3); and a light chain variable domain (VL) comprising:
(d) a light chain (CDR L)1 amino acid sequence of RSSTGAVTTSNYAN (SEQ ID NO:4);
(e) a CDR L2 amino acid sequence of GTNKRAP (SEQ ID NO:5); and
(f) a CDR L3 amino acid sequence of ALWYSNHWV (SEQ ID NO:6).

In one aspect of the use, the antigen binding receptor comprises:
(i) a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof, in particular the CD28 transmembrane domain or a fragment thereof,
(ii) at least one stimulatory signaling domain selected from the group consisting of the intracellular domain of CD3z, of FCGR3A and of NKG2D, or fragments thereof, in particular wherein the at least one stimulatory signaling domain is the CD3z intracellular domain or a fragment thereof, and/or
(iii) at least one co-stimulatory signaling domain individually selected from the group consisting of the intracellular domain of CD27, of CD28, of CD137, of OX40, of ICOS, of DAP10 and of DAP12, or fragments thereof, in particular wherein the at least one co-stimulatory signaling domain is the CD28 intracellular domain or a fragment thereof.

In one aspect, the transduced T cell is administered before, simultaneously with or after administration of the antibody.

Further provided is a kit comprising:
(a) an antibody comprising a heterodimeric Fc domain composed of a first and a second subunit, wherein the first subunit comprises the amino acid mutation P329G according to EU numbering, wherein the second subunit comprises a proline (P) at position 329 according to EU numbering.
(b) a transduced T cell capable of expressing an antigen binding receptor capable of specific binding to the first subunit.

Further provided is a kit comprising:
(a) an antibody comprising a heterodimeric Fc domain composed of a first and a second subunit, wherein the first subunit comprises the amino acid mutation P329G according to EU numbering, wherein the second subunit comprises a proline (P) at position 329 according to EU numbering.
(b) an isolated polynucleotide encoding an antigen binding receptor capable of specific binding to the first subunit.

Further provided is an antibody comprising a heterodimeric Fc domain and an antigen binding receptor substantially as hereinbefore described with reference to any of the Examples or to any one of the accompanying drawings.

### SHORT DESCRIPTION OF THE FIGURES

FIG. 1A, FIG. 1B, FIG. 1C and FIG. 1D: Schematic representation of second generation chimeric antigen binding receptor with anti-P329G binding moiety in the scFv format. In VH x VL scFv (FIG. 1A) orientation and VL x VH (FIG. 1B) orientation. FIG. 1C and FIG. 1D show DNA constructs encoding the antigen binding receptors depicted in FIG. 1A and FIG. 1B, respectively.

FIG. 2A and FIG. 2B: depicted is the CAR surface expression of different humanized scFv variants (FIG. 2A) and the correlating GFP expression serving as transduction control (FIG. 2B).

FIG. 3A, FIG. 3B, FIG. 3C, FIG. 3D, FIG. 3E and FIG. 3F: Evaluation of unspecific signaling of anti-P329G CAR Jurkat reporter T cells employing different humanized versions of the P329G binder as binding moiety. Activation was assessed by quantification of the intensity of CD3 downstream signaling using anti-P329G CAR Jurkat-NFAT reporter assay either in the presence of antibodies possessing different Fc variants or with P329G Fc variants but without target cells. Depicted are technical average values from triplicates, error bars indicate SD.

FIG. 4A, FIG. 4B, FIG. 4C, FIG. 4D, FIG. 4E, FIG. 4F, FIG. 4G, FIG. 4H and FIG. 4I: Activation of anti-P329G CAR Jurkat reporter T cells employing different humanized versions of the P329G binder in the presence of FolR1⁺ target cells with high (HeLa-FolR1), medium (Skov3) and low (HT29) target expression levels in combination with antibodies that possess high (16D5), medium (16D5 W96Y) or low (16D5 G49S/K53A) affinities towards FolR1. Activation was assessed by quantification of the intensity of CD3 downstream signaling using anti-P329G CAR Jurkat-NFAT reporter assay. Depicted are technical average values from triplicates, error bars indicate SD.

FIG. 5A and FIG. 5B: Activation of anti-P329G CAR Jurkat NFAT reporter T cells employing different humanized versions of the P329G binder as binding moiety. Activity of the reporter cells was evaluated in the presence of anti-FolR1 (16D5) P329G IgG1 targeting IgG and HeLa (FolR1) target cells (FIG. 5A). Antibody does-dependent activation was assessed by quantification of the intensity of CD3 downstream signaling using anti-P329G CAR Jurkat-NFAT reporter assay and the area under the curve was calculated (FIG. 5B). Depicted are technical average values from triplicates, error bars indicate SD.

FIG. 6A and FIG. 6B: Activation of anti-P329G CAR Jurkat NFAT reporter T cells employing different humanised versions of the P329G binder as binding moiety. Activity of the reporter cells was evaluated in the presence of anti-HER2 (Pertuzumab) P329G IgG1 targeting IgG and HeLa (HER2+) target cells (FIG. 6A). Antibody does-dependent activation was assessed by quantification of the intensity of CD3 downstream signaling using anti-P329G CAR Jurkat-NFAT reporter assay and the area under the curve was calculated (FIG. 6B). Depicted are technical average values from triplicates, error bars indicate SD.

FIG. 7A and FIG. 7B: depicted is the heterodimeric IgG, generated with knobs into hole technology. FIG. 7A: IgG type antibody according to the invention. One heavy chain comprises a proline at position 329 (numbering according to Kabat) which is the wildtype amino acid at this position. In the other heavy chain the P329G (numbering according to Eu nomenclature) is present. This mutation is known to disrupt FcγR interactions. FIG. 7B: In a further embodiment, the antibody additionally possess an altered glycosylation pattern. Due to the expression cell line non-fucosylated oligosaccharides are present to asparagine 297 in the Fc region (afucosylated Fc). This glycoengineered variant binds with increased affinity to FcgRIII.

FIG. 8A and FIG. 8B: Schematic representation of second generation chimeric antigen binding receptor with anti-P329G binding moiety in the scFv format binding to the P329G mutation in the heterodimeric IgG (FIG. 8A). Schematic representation of second generation chimeric antigen binding receptor with the CD16 extracellular moiety binding to the non-fucosylated olicosaccharides present in the heterodimeric IgG (FIG. 8B).

FIG. 9A and FIG. 9B: Activation of CD16-CAR Jurkat reporter T cells (FIG. 9A) used as ADCC reporter cell line and anti-P329G CAR Jurkat reporter T cells (FIG. 9B) in the presence of WSUDLCL2 CD20⁺ target cells and different concentrations of anti-CD20 heterodimeric IgG1, anti-CD20 P329G LALA IgG1, anti-CD20 glycomodified IgG1 or anti-CD20 wild type IgG1. Activation was assessed by quantification of the intensity of CD3 downstream signaling using the CAR Jurkat-NFAT reporter assay. Depicted are technical average values from triplicates, error bars indicate SD.

FIG. 10: Depicted is the WSUDLCL2 target cell lysis by CD16 CAR T cells in the presence of anti-CD20 heterodimeric IgG1, anti-CD20 P329G LALA IgG1 or anti-CD20 glycomodified IgG1. Depicted are technical duplicates, error bars indicate SD.

FIG. 11A and FIG. 11B: Depicted is a bar diagram that demonstrates the ability of the anti-CD20 heterodimeric IgG1, the anti-CD20 P329G LALA IgG1, the anti CD20 defucosylated IgG1 and the wildtype IgG1 to induce ADCC in a co-culture of WSUDLCL2 (CD20) and PBMCs. Values are calculated from technical triplicates and error bars indicate % SD.

FIG. 12: Activation of NK cells in the presence of anti-CD20 heterodimeric IgG1, anti-CD20 P329G LALA IgG, anti-CD20 defucosylated IgG1 and the wildtype IgG1. The activation of NK cells demonstrated by upregulation of CD107a and downregulation of CD16 receptor. Depicted are technical average values from triplicates, error bars indicate SD.

FIG. 13A and FIG. 13B: Levels of IFN-7, IL-2, TNF-α, IL-6, IL-8 and MCP-1 in a whole blood assay for donor 1 (FIG. 13A) and donor 2 (FIG. 13B) after treatment with anti-CD20 heterodimeric GA101, anti-CD20 P329G LALA GA101, anti-CD20 defucosylated GA101 or anti-CD20 wildtype GA101 (wild type Fc). Fresh whole blood is incubated with escalating concentrations of the different anti-CD20 antibodies. At 24 h serum from the technical duplicates were pooled and the levels of cytokines were measured by Luminex.

### DETAILED DESCRIPTION

### Definitions

Terms are used herein as generally used in the art, unless otherwise defined in the following.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

“Antibody-dependent cell-mediated cytotoxicity” (“ADCC”) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

An “effective amount” of an agent, e.g., a pharmaceutical composition, refers to an amount effective, at dosages and for periods of time necessary, to achieve the desired therapeutic or prophylactic result.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g. hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid. Amino acids may be referred to herein by either their commonly known three letter symbols or by the one-letter symbols recommended by the IUPAC-IUB Biochemical Nomenclature Commission.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G₃₂₉, P329G, or Pro329Gly.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable domain (VL) and an antibody heavy chain variable domain (VH).

As used herein, the term “antigen binding molecule” refers in its broadest sense to a molecule that specifically binds an antigenic determinant. Examples of antigen binding molecules are immunoglobulins and derivatives, e.g., fragments, thereof as well as antigen binding receptors and derivatives thereof.

As used herein, the term “antigen binding moiety” refers to a polypeptide molecule that specifically binds to an antigenic determinant. In one embodiment, an antigen binding moiety is able to direct the entity to which it is attached (e.g. a cell expressing an antigen binding receptor comprising the antigen binding moiety) to a target site, for example to a specific type of tumor cell or tumor stroma bearing the antigenic determinant. Antigen binding moieties include antibodies and fragments thereof as further defined herein. Particular antigen binding moieties include an antigen binding domain of an antibody, comprising an antibody heavy chain variable region and an antibody light chain variable region (e.g. a scFv fragment). In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

In the context of the present invention the term “antigen binding receptor” relates to an antigen binding molecule comprising an anchoring transmembrane domain and an extracellular domain comprising at least one antigen binding moiety. An antigen binding receptor can be made of polypeptide parts from different sources. Accordingly, it may be also understood as a “fusion protein” and/or a “chimeric protein”. Usually, fusion proteins are proteins created through the joining of two or more genes (or preferably cDNAs) that originally coded for separate proteins. Translation of this fusion gene (or fusion cDNA) results in a single polypeptide, preferably with functional properties derived from each of the original proteins. Recombinant fusion proteins are created artificially by recombinant DNA technology for use in biological research or therapeutics. Further details to the antigen binding receptors of the present invention are described herein below. In the context of the present invention a CAR (chimeric antigen receptor) is understood to be an antigen binding receptor comprising an extracellular portion comprising an antigen binding moiety fused by a spacer sequence to an anchoring transmembrane domain which is itself fused to intracellular signaling domains.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

The term “antigen binding domain” refers to the part of an antibody or an antigen binding receptor that comprises the area which specifically binds to and is complementary to part or all of an antigen. An antigen binding domain may be provided by, for example, one or more immunoglobulin variable domains (also called variable regions). Particularly, an antigen binding domain comprises an immunoglobulin light chain variable domain (VL) and an immunoglobulin heavy chain variable domain (VH).

As used herein, the term “antigenic determinant” is synonymous with “antigen” and “epitope” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

“Antibodies comprising a heterodimeric Fc domain” according to the present invention may have one, two, three or more binding domains and may be monospecific, bispecific or multispecific. The antibodies can be full length from a single species, or be chimerized or humanized. For an antibody with more than two antigen binding domains, some binding domains may be identical and/or have the same specificity.

The term “ATD” as used herein refers to “anchoring transmembrane domain” which defines a polypeptide stretch capable of integrating in (the) cellular membrane(s) of a cell. The ATM can be fused to extracellular and/or intracellular polypeptide domains wherein these extracellular and/or intracellular polypeptide domains will be confined to the cell membrane. In the context of the antigen binding receptors of the present invention the ATM confers membrane attachment and confinement of the antigen binding receptor of the present invention. The antigen binding receptors of the present invention comprise at least one ATM and an extracellular domain comprising an antigen binding moiety. Additionally, the ATM may be fused to intracellular signaling domains.

By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) Of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M).

The term “CDR” as employed herein relates to “complementary determining region”, which is well known in the art. The CDRs are parts of immunoglobulins or antigen binding receptors that determine the specificity of said molecules and make contact with a specific ligand. The CDRs are the most variable part of the molecule and contribute to the antigen binding diversity of these molecules. There are three CDR regions CDR1, CDR2 and CDR3 in each V domain. CDR-H depicts a CDR region of a variable heavy chain and CDR-L relates to a CDR region of a variable light chain. VH means the variable heavy chain and VL means the variable light chain. The CDR regions of an Ig-derived region may be determined as described in “Kabat” (Sequences of Proteins of Immunological Interest”, 5th edit. NIH Publication no. 91-3242 U.S. Department of Health and Human Services (1991); Chothia J. Mol. Biol. 196 (1987), 901-917) or “Chothia” (Nature 342 (1989), 877-883).

The term “CD3z” refers to T-cell surface glycoprotein CD3 zeta chain, also known as “T-cell receptor T3 zeta chain” and “CD247”.

The term “chimeric antibody” refers to an antibody in which a portion of the heavy and/or light chain is derived from a particular source or species, while the remainder of the heavy and/or light chain is derived from a different source or species.

The term “chimeric antigen receptor” or “chimeric receptor” or “CAR” refers to an antigen binding receptor constituted of an extracellular portion of an antigen binding moiety (e.g. a single chain antibody domain) fused by a spacer sequence to intracellular signaling/co-signalling domains (such as e.g. of CD3z and CD28).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. The heavy chain constant domains that correspond to the different classes of immunoglobulins are called α, δ, δ, γ, and μ, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

By a “crossover” Fab molecule (also termed “Crossfab”) is meant a Fab molecule wherein the variable domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the crossover Fab molecule.

The term “CSD” as used herein refers to co-stimulatory signaling domain.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

A “Fab molecule” refers to a protein consisting of the VH and CH1 domain of the heavy chain (the “Fab heavy chain”) and the VL and CL domain of the light chain (the “Fab light chain”) of an immunoglobulin.

The term “Fc domain” or “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

By “fused” is meant that the components (e.g., a Fab and a transmembrane domain) are linked by peptide bonds, either directly or via one or more peptide linkers.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “heterodimeric” Fc domain as herein described refers to an Fc domain composed of two non-identical subunits. For example one of the Fc domain subunits may comprise a mutation whereas the other Fc domain subunit does not comprise the (same) mutation.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed below for polypeptides (e.g., ALIGN-2).

By an “isolated polypeptide” or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017, doi:10.1038/nm.4356 or EP 2 101 823 B1).

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

The term “polypeptide” refers to any chain of two or more amino acids, and does not refer to a specific length of the product. Thus, peptides, dipeptides, tripeptides, oligopeptides, “protein”, “amino acid chain”, or any other term used to refer to a chain of two or more amino acids, are included within the definition of “polypeptide”, and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

The term “polynucleotide” refers to an isolated nucleic acid molecule or construct, e.g., messenger RNA (mRNA), virally-derived RNA, or plasmid DNA (pDNA). A polynucleotide may comprise a conventional phosphodiester bond or a non-conventional bond (e.g., an amide bond, such as found in peptide nucleic acids (PNA). The term nucleic acid molecule refers to any one or more nucleic acid segments, e.g., DNA or RNA fragments, present in a polynucleotide.

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

The term “regulatory sequence” refers to DNA sequences, which are necessary to effect the expression of coding sequences to which they are ligated. The nature of such control sequences differs depending upon the host organism. In prokaryotes, control sequences generally include promoter, ribosomal binding site, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule. In a preferred embodiment, the antigen binding moiety is a scFv fragment.

The term “SSD” as used herein refers to “stimulatory signaling domain”.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The immune activating Fc domain binding molecules of the invention are capable of inducing T cell activation. Suitable assays to measure T cell activation are known in the art described herein.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

The term “valent” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### Compositions and Methods

In one aspect, the invention is based, in part, on antibodies comprising a heterodimeric Fc domain. In certain aspects, antibodies comprising the amino acid mutation P329G according to EU numbering are provided. In particular, the invention provided antibodies comprising the amino acid mutation P329G according to EU numbering in one of the two Fc domain subunits. Antibodies of the invention are useful, e.g., for the treatment of cancer.

Recruiting innate immune cells at the same time with CAR-T cells may inter alia help to reduce adverse events (e.g. cytokine release syndrome) by giving first the antibody and infusing the CAR-T cells only at a later time point when the antibody has already induced ADCC-mediated anti-tumor efficacy and debulking. Furthermore, recruiting innate immune cells at the same time with CAR-T cells may inter alia help in generating a secondary immune response by activating antigen presenting cells such as FcgR expressing monocytes, macrophages and dendritic cells in the tumor microenvironment.

The herein provided antibodies comprise a heterodimeric Fc domain (e.g. a human IgG1) Fc region comprising the P329G mutation according to EU numbering.

The P329G mutation reduces binding to Fcγ receptors and associated effector function. A mutated Fc domain comprising the P329G mutation, in particular in both Fc domain subunits, binds to Fcγ receptors with reduced or abolished affinity compared to the non-mutated Fc domain. However, Fcγ receptors mediated receptor function might be desired as herein above described.

According to the present invention, provided is an antibody comprising a heterodimeric Fc domain composed of a first and a second subunit, wherein the first subunit comprises the amino acid mutation P329G according to EU numbering and wherein the second subunit comprises a proline (P) at position 329 according to EU numbering. In one embodiment, the Fc domain is an IgG, particularly an IgG₁, Fc domain. In one embodiment, the Fc domain is a human Fc domain.

Antibodies comprising heterodimeric Fc domains according to the invention comprise different subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of (multispecific, e.g. bispecific) antibodies in recombinant production, it will thus be advantageous to introduce in the heterodimeric Fc domain of the (multispecific, e.g. bispecific) antibody further modifications promoting the association of the desired polypeptides.

Accordingly, in preferred aspects, the Fc domain of the (multispecific, e.g. bispecific) antibody according to the invention comprises a modification promoting the association of the first and the second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

There exist several approaches for modifications in the CH3 domain of the Fc domain in order to enforce heterodimerization, which are well described e.g. in WO 96/27011, WO 98/050431, EP 1870459, WO 2007/110205, WO 2007/147901, WO 2009/089004, WO 2010/129304, WO 2011/90754, WO 2011/143545, WO 2012058768, WO 2013157954, WO 2013096291. Typically, in all such approaches the CH3 domain of the first subunit of the Fc domain and the CH3 domain of the second subunit of the Fc domain are both engineered in a complementary manner so that each CH3 domain (or the heavy chain comprising it) can no longer homodimerize with itself but is forced to heterodimerize with the complementarily engineered other CH3 domain (so that the first and second CH3 domain heterodimerize and no homodimers between the two first or the two second CH3 domains are formed). These different approaches for improved heavy chain heterodimerization are contemplated as different alternatives in combination with the heavy-light chain modifications (e.g. VH and VL exchange/replacement in one binding arm and the introduction of substitutions of charged amino acids with opposite charges in the CH1/CL interface) in the (multispecific, e.g. bispecific) antibody which reduce heavy/light chain mispairing and Bence Jones-type side products.

In a specific aspect said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a preferred aspect, in the CH3 domain of the first subunit of the Fc domain of the (multispecific, e.g. bispecific) antibody an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

Preferably said amino acid residue having a larger side chain volume is selected from the group consisting of arginine (R), phenylalanine (F), tyrosine (Y), and tryptophan (W).

Preferably said amino acid residue having a smaller side chain volume is selected from the group consisting of alanine (A), serine (S), threonine (T), and valine (V).

The protuberance and cavity can be made by altering the nucleic acid encoding the polypeptides, e.g. by site-specific mutagenesis, or by peptide synthesis.

In a specific aspect, in (the CH3 domain of) the first subunit of the Fc domain (the “knobs” subunit) the threonine residue at position 366 is replaced with a tryptophan residue (T366W), and in (the CH3 domain of) the second subunit of the Fc domain (the “hole” subunit) the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A) (numberings according to Kabat EU index).

In yet a further aspect, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numberings according to Kabat EU index). Introduction of these two cysteine residues results in formation of a disulfide bridge between the two subunits of the Fc domain, further stabilizing the dimer (Carter, J Immunol Methods 248, 7-15 (2001)).

In a preferred aspect, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In a preferred aspect the antigen binding domain that binds to CD3 is fused (optionally via the second antigen binding domain, which binds to a second antigen (i.e. FolR1), and/or a peptide linker) to the first subunit of the Fc domain (comprising the “knob” modification). Without wishing to be bound by theory, fusion of the antigen binding domain that binds CD3 to the knob-containing subunit of the Fc domain will (further) minimize the generation of antibodies comprising two antigen binding domains that bind to CD3 (steric clash of two knob-containing polypeptides).

Other techniques of CH3-modification for enforcing the heterodimerization are contemplated as alternatives according to the invention and are described e.g. in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291.

In one aspect, the heterodimerization approach described in EP 1870459, is used alternatively. This approach is based on the introduction of charged amino acids with opposite charges at specific amino acid positions in the CH3/CH3 domain interface between the two subunits of the Fc domain. A particular aspect for the (multispecific) antibody of the invention are amino acid mutations R409D; K370E in one of the two CH3 domains (of the Fc domain) and amino acid mutations D399K; E357K in the other one of the CH3 domains of the Fc domain (numbering according to Kabat EU index).

In another aspect, the (multispecific, e.g. bispecific) antibody of the invention comprises amino acid mutation T366W in the CH3 domain of the first subunit of the Fc domain and amino acid mutations T366S, L368A, Y407V in the CH3 domain of the second subunit of the Fc domain, and additionally amino acid mutations R409D; K370E in the CH3 domain of the first subunit of the Fc domain and amino acid mutations D399K; E357K in the CH3 domain of the second subunit of the Fc domain (numberings according to Kabat EU index).

In another aspect, the (multispecific, e.g. bispecific) antibody of the invention comprises amino acid mutations S354C, T366W in the CH3 domain of the first subunit of the Fc domain and amino acid mutations Y349C, T366S, L368A, Y407V in the CH3 domain of the second subunit of the Fc domain, or said (multispecific, e.g. bispecific) antibody comprises amino acid mutations Y349C, T366W in the CH3 domain of the first subunit of the Fc domain and amino acid mutations S354C, T366S, L368A, Y407V in the CH3 domains of the second subunit of the Fc domain and additionally amino acid mutations R409D; K370E in the CH3 domain of the first subunit of the Fc domain and amino acid mutations D399K; E357K in the CH3 domain of the second subunit of the Fc domain (all numberings according to Kabat EU index).

In one aspect, the heterodimerization approach described in WO 2013/157953 is used alternatively. In one aspect, a first CH3 domain comprises amino acid mutation T366K and a second CH3 domain comprises amino acid mutation L351D (numberings according to Kabat EU index). In a further aspect, the first CH3 domain comprises further amino acid mutation L351K. In a further aspect, the second CH3 domain comprises further an amino acid mutation selected from Y349E, Y349D and L368E (particularly L368E) (numberings according to Kabat EU index).

In one aspect, the heterodimerization approach described in WO 2012/058768 is used alternatively. In one aspect a first CH3 domain comprises amino acid mutations L351Y, Y407A and a second CH3 domain comprises amino acid mutations T366A, K409F. In a further aspect the second CH3 domain comprises a further amino acid mutation at position T411, D399, 5400, F405, N390, or K392, e.g. selected from a) T411N, T411R, T411Q, T411K, T411D, T411E or T411W, b) D399R, D399W, D399Y or D399K, c) S400E, S400D, S400R, or S400K, d) F405I, F405M, F405T, F405S, F405V or F405W, e) N390R, N390K or N390D, f) K392V, K392M, K392R, K392L, K392F or K392E (numberings according to Kabat EU index). In a further aspect a first CH3 domain comprises amino acid mutations L351Y, Y407A and a second CH3 domain comprises amino acid mutations T366V, K409F. In a further aspect, a first CH3 domain comprises amino acid mutation Y407A and a second CH3 domain comprises amino acid mutations T366A, K409F. In a further aspect, the second CH3 domain further comprises amino acid mutations K392E, T411E, D399R and S400R (numberings according to Kabat EU index).

In one aspect, the heterodimerization approach described in WO 2011/143545 is used alternatively, e.g. with the amino acid modification at a position selected from the group consisting of 368 and 409 (numbering according to Kabat EU index).

In one aspect, the heterodimerization approach described in WO 2011/090762, which also uses the knobs-into-holes technology described above, is used alternatively. In one aspect a first CH3 domain comprises amino acid mutation T366W and a second CH3 domain comprises amino acid mutation Y407A. In one aspect, a first CH3 domain comprises amino acid mutation T366Y and a second CH3 domain comprises amino acid mutation Y407T (numberings according to Kabat EU index).

In one aspect, the (multispecific, e.g. bispecific) antibody or its Fc domain is of IgG₂ subclass and the heterodimerization approach described in WO 2010/129304 is used alternatively.

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable. In one such aspect, a first CH3 domain comprises amino acid substitution of K392 or N392 with a negatively charged amino acid (e.g. glutamic acid (E), or aspartic acid (D), particularly K392D or N392D) and a second CH3 domain comprises amino acid substitution of D399, E356, D356, or E357 with a positively charged amino acid (e.g. lysine (K) or arginine (R), particularly D399K, E356K, D356K, or E357K, and more particularly D399K and E356K). In a further aspect, the first CH3 domain further comprises amino acid substitution of K409 or R409 with a negatively charged amino acid (e.g. glutamic acid (E), or aspartic acid (D), particularly K409D or R409D). In a further aspect the first CH3 domain further or alternatively comprises amino acid substitution of K439 and/or K370 with a negatively charged amino acid (e.g. glutamic acid (E), or aspartic acid (D)) (all numberings according to Kabat EU index).

In yet a further aspect, the heterodimerization approach described in WO 2007/147901 is used alternatively. In one aspect, a first CH3 domain comprises amino acid mutations K253E, D282K, and K322D and a second CH3 domain comprises amino acid mutations D239K, E240K, and K292D (numberings according to Kabat EU index).

In still another aspect, the heterodimerization approach described in WO 2007/110205 can be used alternatively.

In one aspect, the first subunit of the Fc domain comprises amino acid substitutions K392D and K409D, and the second subunit of the Fc domain comprises amino acid substitutions D356K and D399K (numbering according to Kabat EU index).

In certain aspects, an antibody provided herein is altered to increase or decrease the extent to which the antibody is glycosylated. Addition or deletion of glycosylation sites to an antibody may be accomplished by altering the amino acid sequence such that one or more glycosylation sites is created or removed.

Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

The herein provided antibodies comprise an Fc domain (e.g. a human IgG1) Fc region comprising the P329G mutation according to EU numbering. In certain aspects, one or more additional amino acid modifications may be introduced into the Fc region of an antibody provided herein. The Fc region variant may comprise a human Fc region sequence (e.g., a human IgG₁ Fc region) comprising an amino acid modification (e.g., a substitution) at one or more amino acid positions.

In certain aspects, an heterodimeric antibody variant comprises an Fc region with one or more amino acid substitutions, which increase FcRn binding. In one embodiment the mutated Fc domain exhibits increased binding affinity to an Fc receptor and/or reduced effector function, as compared to a native IgG₁ Fc domain. In one embodiment the Fc domain comprises one or more amino acid mutations that increase binding to an Fc receptor and/or effector function.

In certain aspects, an antibody variant comprises an Fc region with one or more amino acid substitutions which improve ADCC, e.g., substitutions at positions 298, 333, and/or 334 of the Fc region (EU numbering of residues). In vitro and/or in vivo cytotoxicity assays can be conducted to confirm the increase of CDC and/or ADCC activities. For example, Fc receptor (FcR) binding assays can be conducted to ensure that the antibody has improved FcγR binding (hence likely improved ADCC activity). The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96© non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some aspects, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues 1253, H310, H433, N434, and H435 (EU numbering of residues) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### Antigen Binding Moiety

In one aspect, the antigen binding moiety is a scFv, a Fab, a crossFab or a scFab, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain.”

In a further aspect, the antigen binding moiety is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antigen binding moiety fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antigen binding moiety is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

In another aspect, the antigen binding moiety is a crossFab. By a “crossover Fab molecule” (also termed “crossFab” or “crossover Fab fragment”) is meant a Fab molecule wherein either the variable regions or the constant regions of the Fab heavy and light chain are exchanged, i.e. the crossFab fragment comprises a peptide chain composed of the light chain variable region and the heavy chain constant region, and a peptide chain composed of the heavy chain variable region and the light chain constant region. Accordingly, a crossFab fragment comprises a polypeptide composed of the heavy chain variable and the light chain constant regions (VH-CL), and a polypeptide composed of the light chain variable and the heavy chain constant regions (VL-CH1). For clarity, the polypeptide chain comprising the heavy chain constant region is referred to herein as the heavy chain and the polypeptide chain comprising the light chain constant regions is referred to herein as the light chain of the crossFab fragment.

### Target Cell Antigens

The herein provided antigen binding moiety has specificity for a target cell surface molecule, e.g. a tumor-specific antigen that naturally occurs on the surface of a tumor cell. In the context of the present invention, such antibodies comprising such antigen binding moieties will bring transduced T cells as described herein in physical contact with a target cell (e.g. a tumor cell), wherein the transduced T cell becomes activated. Activation of transduced T cells of the present invention preferentially results in lysis of the target cell as described herein.

Examples of target cell antigens (e.g., tumor markers) that naturally occur on the surface of target (e.g. tumor) cells are given herein below and comprise, but are not limited to FAP (fibroblast activation protein), CEA (carcinoembryonic antigen), p95 (p95HER2), BCMA (B-cell maturation antigen), EpCAM (epithelial cell adhesion molecule), MSLN (mesothelin), MCSP (melanoma chondroitin sulfate proteoglycan), HER-1 (human epidermal growth factor 1), HER-2 (human epidermal growth factor 2), HER-3 (human epidermal growth factor 3), CD19, CD20, CD22, CD33, CD38, CD52Flt3, folate receptor 1 (FOLR1), human trophoblast cell-surface antigen 2 (Trop-2) cancer antigen 12-5 (CA-12-5), human leukocyte antigen-antigen D related (HLA-DR), MUC-1 (Mucin-1), A33-antigen, PSMA (prostate-specific membrane antigen), FMS-like tyrosine kinase 3 (FLT-3), PSMA (prostate specific membrane antigen), PSCA (prostate stem cell antigen), transferrin-receptor, TNC (tenascin), carbon anhydrase IX (CA-IX), and/or peptides bound to a molecule of the human major histocompatibility complex (MHC).

The sequences of the above mentioned antigens are available in the UniProtKB/Swiss-Prot database and can be retrieved from http://www.uniprot.org/uniprot/?query=reviewed %3Ayes. These (protein) sequences also relate to annotated modified sequences. The present invention also provides techniques and methods wherein homologous sequences, and also genetic allelic variants and the like of the concise sequences provided herein are used. Preferably such variants and the like of the concise sequences herein are used. Preferably, such variants are genetic variants. The skilled person may easily deduce the relevant coding region of these (protein) sequences in these databank entries, which may also comprise the entry of genomic DNA as well as mRNA/cDNA. The sequence(s) of the (human) FAP (fibroblast activation protein) can be obtained from the Swiss-Prot database entry Q12884 (entry version 168, sequence version 5); The sequence(s) of the (human) CEA (carcinoembryonic antigen) can be obtained from the Swiss-Prot database entry P06731 (entry version 171, sequence version 3); the sequence(s) of the (human) EpCAM (Epithelial cell adhesion molecule) can be obtained from the Swiss-Prot database entry P16422 (entry version 117, sequence version 2); the sequence(s) of the (human) MSLN (mesothelin) can be obtained from the UniProt Entry number Q13421 (version number 132; sequence version 2); the sequence(s) of the (human) FMS-like tyrosine kinase 3 (FLT-3) can be obtained from the Swiss-Prot database entry P36888 (primary citable accession number) or Q13414 (secondary accession number) with the version number 165 and the sequence version 2; the sequences of (human) MCSP (melanoma chondroitin sulfate proteoglycan) can be obtained from the UniProt Entry number Q6UVK1 (version number 118; sequence version 2); the sequence(s) of the (human) folate receptor 1 (FOLR1) can be obtained from the UniProt Entry number P15328 (primary citable accession number) or Q53EW2 (secondary accession number) with the version number 153 and the sequence version 3; the sequence(s) of the (human) trophoblast cell-surface antigen 2 (Trop-2) can be obtained from the UniProt Entry number P09758 (primary citable accession number) or Q15658 (secondary accession number) with the version number 172 and the sequence version 3; the sequence(s) of the (human) PSCA (prostate stem cell antigen) can be obtained from the UniProt Entry number 043653 (primary citable accession number) or Q6UW92 (secondary accession number) with the version number 134 and the sequence version 1; the sequence(s) of the (human) HER-1 (Epidermal growth factor receptor) can be obtained from the Swiss-Prot database entry P00533 (entry version 177, sequence version 2); the sequence(s) of the (human) HER-2 (Receptor tyrosine-protein kinase erbB-2) can be obtained from the Swiss-Prot database entry P04626 (entry version 161, sequence version 1); the sequence(s) of the (human) HER-3 (Receptor tyrosine-protein kinase erbB-3) can be obtained from the Swiss-Prot database entry P21860 (entry version 140, sequence version 1); the sequence(s) of the (human) CD20 (B-lymphocyte antigen CD20) can be obtained from the Swiss-Prot database entry P11836 (entry version 117, sequence version 1); the sequence(s) of the (human) CD22 (B-lymphocyte antigen CD22) can be obtained from the Swiss-Prot database entry P20273 (entry version 135, sequence version 2); the sequence(s) of the (human) CD33 (B-lymphocyte antigen CD33) can be obtained from the Swiss-Prot database entry P20138 (entry version 129, sequence version 2); the sequence(s) of the (human) CA-12-5 (Mucin 16) can be obtained from the Swiss-Prot database entry Q8WXI7 (entry version 66, sequence version 2); the sequence(s) of the (human) HLA-DR can be obtained from the Swiss-Prot database entry Q29900 (entry version 59, sequence version 1); the sequence(s) of the (human) MUC-1 (Mucin-1) can be obtained from the Swiss-Prot database entry P15941 (entry version 135, sequence version 3); the sequence(s) of the (human) A33 (cell surface A33 antigen) can be obtained from the Swiss-Prot database entry Q99795 (entry version 104, sequence version 1); the sequence(s) of the (human) PSMA (Glutamate carboxypeptidase 2) can be obtained from the Swiss-Prot database entry Q04609 (entry version 133, sequence version 1), the sequence(s) of the (human) transferrin receptor can be obtained from the Swiss-Prot database entries Q9UP52 (entry version 99, sequence version 1) and P02786 (entry version 152, sequence version 2); the sequence of the (human) TNC (tenascin) can be obtained from the Swiss-Prot database entry P24821 (entry version 141, sequence version 3); or the sequence(s) of the (human) CA-IX (carbonic anhydrase IX) can be obtained from the Swiss-Prot database entry Q16790 (entry version 115, sequence version 2).

In a preferred embodiment, the target cell antigen is selected from the group consisting of fibroblast activation protein (FAP), carcinoembryonic antigen (CEA), mesothelin (MSLN), CD20, folate receptor 1 (FOLR1), and tenascin (TNC).

Antigen binding moieties (e.g. a scFv, a Fab, a crossFab or a scFab) capable of specific binding to any of the above mentioned target cell antigens can be generated using methods well known in the art such as immunizing a mammalian immune system and/or phage display using recombinant libraries.

### Library-Derived Antigen Binding Moieties

In certain aspects, an antigen binding moiety provided herein is derived from a library. Antigen binding moieties of the invention may be isolated by screening combinatorial libraries for antigen binding moieties with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antigen binding moieties possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, N J, 2001) and in Marks and Bradbury in Methods in Molecular Biology 248:161-175 (Lo, ed., Human Press, Totowa, N J, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antigen binding moieties to the immunogen without the requirement of constructing hybridomas. Alternatively, the naive repertoire can be cloned (e.g., from human) to provide a single source of antigen binding moieties to a wide range of non-self and also self antigens without any immunization as described by Griffiths et al. in EMBO Journal 12: 725-734 (1993). Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936.

Further examples of methods known in the art for screening combinatorial libraries for antigen binding moieties with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Antigen binding moieties or antibody fragments isolated from human antibody libraries are considered human antibodies or human antibody fragments herein.

### Affinity

In certain aspects, an antigen binding moiety provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In one aspect, K_{D} is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one aspect, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 10⁶ M⁻¹ s−1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In an alternative method, K_{D} is measured by a radiolabeled antigen binding assay (RIA). In one aspect, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

### Chimeric and Humanized Antibodies

In certain aspects, a heterodimeric antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In certain aspects, a heterodimeric antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### Multispecific Antibodies

In certain aspects, a heterodimeric antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); crosslinking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Hence, in certain aspects, an antibody provided herein is a multispecific antibody, particularly a bispecific antibody.

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### Antibody Variants

In certain aspects, amino acid sequence variants of the heterodimeric antibodies provided herein are contemplated. For example, it may be desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### Substitution, Insertion, and Deletion Variants

In certain aspects, antibody variants having one or more amino acid substitutions are provided. Sites of interest for substitutional mutagenesis include the CDRs and FRs. Conservative substitutions are shown in Table 1 under the heading of “preferred substitutions”. More substantial changes are provided in Table 1 under the heading of“exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 8,30,930, 7,855,275, 9,000,130, or WO 2016040856.

### Antibody Derivatives

In certain aspects, a heterodimeric antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

Exemplary heterodimeric Antibodies

In one aspect, the invention provides heterodimeric antibodies that bind to CD20. In one aspect, provided are isolated heterodimeric antibodies that bind to CD20. In one aspect, the invention provides heterodimeric antibodies that specifically bind to CD20. In one aspect, the heterodimeric anti-CD20 antibody is humanized. In a further aspect of the invention, a heterodimeric anti-CD20 antibody according to any of the above aspects is a monoclonal antibody, including a chimeric, humanized or human antibody. In one embodiment the heterodimeric anti-CD20 antibody comprises a polypeptide sequence that is at least about 95%, 96%, 97%, 98%, 99% or 100% identical to SEQ ID NO: 129, a polypeptide sequence that is at least about 95%, 96%, 97%, 98%, 99% or 100% identical to SEQ ID NO: 130 and a polypeptide sequence that is at least about 95%, 96%, 97%, 98%, 99% or 100% identical to SEQ ID NO: 131.

In another aspect, any one of the above exemplary heterodimeric antibodies is a full length antibody. In one aspect, additionally the C-terminal glycine (Gly446) is present. In one aspect, additionally the C-terminal glycine (Gly446) and the C-terminal lysine (Lys447) is present

### Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

In case of a bispecific antibody with heterodimeric heavy chains four nucleic acids are required, one for the first light chain, one for the first heavy chain comprising the first heteromonomeric Fc-region polypeptide, one for the second light chain, and one for the second heavy chain comprising the second heteromonomeric Fc-region polypeptide. The four nucleic acids can be comprised in one or more nucleic acid molecules or expression vectors. Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

In one aspect, isolated nucleic acids encoding an antibody as used in the methods as reported herein are provided.

In one aspect, a method of making an antibody comprising a heterodimeric Fc domain is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of an antibody comprising a heterodimeric Fc domain, nucleic acids encoding the antibody, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### Pharmaceutical Compositions

In a further aspect, provided are pharmaceutical compositions comprising any of the antibodies provided herein, e.g., for use in any of the below therapeutic methods. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Pharmaceutical compositions of an antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody compositions are described in U.S. Pat. No. 6,267,958. Aqueous antibody compositions include those described in U.S. Pat. No. 6,171,586 and WO 2006/044908, the latter compositions including a histidine-acetate buffer.

The pharmaceutical composition herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Pharmaceutical compositions for sustained-release may be prepared. Suitable examples of sustained-release preparations include semipermeable matrices of solid hydrophobic polymers containing the antibody, which matrices are in the form of shaped articles, e.g., films, or microcapsules.

The pharmaceutical compositions to be used for in vivo administration are generally sterile. Sterility may be readily accomplished, e.g., by filtration through sterile filtration membranes.

### Therapeutic Methods and Routes of Administration

Any of the heterodimeric antibodies provided herein may be used in therapeutic methods. The heterodimeric antibodies of the invention are combined with antigen binding receptors capable of specific binding to the mutated Fc domain as herein described.

In one aspect, a heterodimeric antibody for use as a medicament is provided. In further aspects, a heterodimeric antibody for use in treating cancer is provided. In certain aspects, heterodimeric antibody for use in a method of treatment is provided. In certain aspects, the invention provides a heterodimeric antibody for use in a method of treating an individual having cancer comprising administering to the individual an effective amount of the heterodimeric antibody. In one such aspect, the method further comprises administering to the individual an effective amount of at least one additional therapeutic agent (e.g., one, two, three, four, five, or six additional therapeutic agents), e.g., as described below. In further aspects, the invention provides a heterodimeric antibody for use in treatment of cancer, in particular cancer of epithelial, endothelial or mesothelial origin and cancer of the blood. In certain aspects, the invention provides a heterodimeric antibody for use in a method of treating cancer, in particular cancer of epithelial, endothelial or mesothelial origin and cancer of the blood in an individual comprising administering to the individual an effective amount of the heterodimeric antibody to treat the cancer. An “individual” according to any of the above aspects is preferably a human.

In a further aspect, the invention provides for the use of a heterodimeric antibody in the manufacture or preparation of a medicament. In one aspect, the medicament is for treatment of cancer. In a further aspect, the medicament is for use in a method of treating cancer comprising administering to an individual having cancer an effective amount of the medicament. In one such aspect, the method further comprises administering to the individual an effective amount of at least one additional therapeutic agent, e.g., as described below. In a further aspect, the medicament is for treatment of cancer, in particular cancer of epithelial, endothelial or mesothelial origin and cancer of the blood. In a further aspect, the medicament is for use in a method of treating cancer, in particular cancer of epithelial, endothelial or mesothelial origin and cancer of the blood in an individual comprising administering to the individual an effective amount of the medicament to treat the cancer. An “individual” according to any of the above aspects may be a human.

In a further aspect, the invention provides a method for treating a cancer. In one aspect, the method comprises administering to an individual having such cancer an effective amount of a heterodimeric antibody. In one such aspect, the method further comprises administering to the individual an effective amount of at least one additional therapeutic agent, as described below.

An “individual” according to any of the above aspects may be a human.

In a further aspect, the invention provides pharmaceutical compositions comprising any of the heterodimeric antibodies provided herein, e.g., for use in any of the above therapeutic methods. In one aspect, a pharmaceutical composition comprises any of the heterodimeric antibodies provided herein and a pharmaceutically acceptable carrier. In another aspect, a pharmaceutical composition comprises any of the heterodimeric antibodies provided herein and at least one additional therapeutic agent, e.g., as described below.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every three weeks (e.g., such that the patient receives from about two to about twenty, or, e.g., about six doses of the antibody). An initial higher loading dose, followed by one or more lower doses may be administered. The progress of this therapy is easily monitored by conventional techniques and assays.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Combination with Antigen Binding Receptors

The heterodimeric antibodies according to the present invention can be combined with cells expressing antigen binding receptors capable of specific binding to the mutated Fc subunit (e.g. comprising the amino acid mutation P329G according to EU numbering) for increased pharmacological activity (as also further described below). Such combination therapies noted above encompass combined administration (where the heterodimeric antibodies and cells are included in the same or separate pharmaceutical compositions), and separate administration, in which case, administration of the heterodimeric antibody of the invention can occur prior to, simultaneously, and/or following, administration of the cells expressing the antigen binding receptors as herein below described.

As herein described, the antibodies according to the present invention are able to efficiently recruit anti-P329G CAR-T cells for killing. Furthermore, the antibodies according to the present invention are able to efficiently recruit innate immune cells such as NK cells or monocytes for FcgR dependent ADCC without unspecific cross-activation.

Recruiting innate immune cells at the same time with CAR-T cells may inter alia help to reduce adverse events (e.g. cytokine release syndrome) by giving first the antibody and infusing the CAR-T cells only at a later time point when the antibody has already induced ADCC-mediated anti-tumor efficacy and debulking. Furthermore, recruiting innate immune cells at the same time with CAR-T cells may inter alia help in generating a secondary immune response by activating antigen presenting cells such as FcgR expressing monocytes, macrophages and dendritic cells in the tumor microenvironment.

In one aspect, administration of the heterodimeric antibody and administration of the cells occur within about one month, or within about one, two or three weeks, or within about one, two, three, four, five, or six days, of each other. In one aspect, the heterodimeric antibody and cells are administered to the patient on Day 1 of the treatment.

The antigen binding receptors of the present invention comprise an extracellular domain comprising at least one antigen binding moiety capable of specific binding to the mutated Fc domain but not capable of specific binding to the parent non-mutated Fc domain. In preferred embodiments, the antigen binding moiety of the antigen binding receptor is a humanized or human antigen binding moiety, e.g. a humanized or human scFv.

The present invention further relates to the transduction of T cells, such as CD8+ T cells, CD4+ T cells, CD3+ T cells, 76 T cells or natural killer (NK) T cells, preferably CD8+ T cells, with the herein provided antigen binding receptor their targeted recruitment, e.g., to a tumor, by the antibody provided herein.

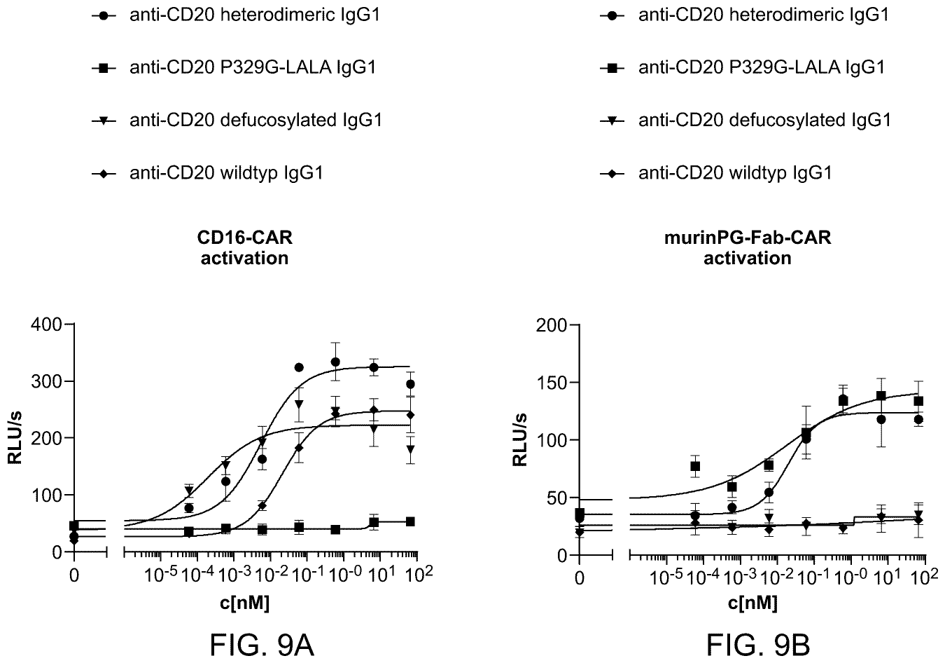

As shown in the appended Examples, the antigen binding receptor comprising an anchoring transmembrane domain and a humanized extracellular domain according to the invention (SEQ ID NO:7 as encoded by the DNA sequence shown in SEQ ID NO:20) was constructed which is capable of specific binding to a therapeutic antibody (represented by the heterodimeric anti-CD20 antibody comprising a heavy chain of SEQ ID NO ID: 129 (comprising the P329G mutation), a heavy chain of SEQ ID NO:130 and two light chains of SEQ ID NO:131). Transduced T cells (Jurkat NFAT T cells) expressing the VH3VL1-CD8ATD-CD137CSD-CD3zSSD fusion protein (SEQ ID NO:7 as encoded by the DNA sequence shown in SEQ ID NO:20) could be strongly activated by co-incubation with the anti-CD20 antibody comprising the P329G mutation in the Fc domain together with CD20 positive tumor cells (see for example FIG. 9B). Additionally, and surprisingly, ADCC effector function as evidenced by CD16-CAR activation (see for example FIG. 9A) could be strongly activated by the heterodimeric anti-CD20 antibody.

Furthermore, the treatment of tumor cells by the combination of an antibody directed against a tumor antigen wherein the antibody comprises the P329G mutation together with transduced T cells expressing the VH3VL1-CD8ATD-CD137CSD-CD3zSSD fusion protein (SEQ ID NO:7 as encoded by the DNA sequence shown in SEQ ID NO:20) surprisingly leads to stronger activation of the transduced T cell compared to the transduced T cells expressing the VL1VH3-CD8ATD-CD137CSD-CD3zSSD (SEQ ID NO:31 as encoded by the DNA sequence shown in SEQ ID NO:33).

In the VH3VL1-CD8ATD-CD137CSD-CD3zSSD fusion protein, the VH domain (V13) is fused at its C-terminus to the N-terminus of the VL domain (VL1) through a peptide linker to form a scFv. The scFv is fused at its C-terminus (the C-terminus of the VL domain) through a peptide linker to the anchoring transmembrane domain (ATD). On the other hand, the VL1VH3-CD8ATD-CD137CSD-CD3zSSD fusion protein, the VL domain (VL1) is fused at its C-terminus to the N-terminus of the VH domain (V13) through a peptide linker to form a scFv. The scFv is fused at its C-terminus (the C-terminus of the VH domain) through a peptide linker to the anchoring transmembrane domain (ATD). Without being bound to theory, the observation that the VH3VL1-CD8ATD-CD137CSD-CD3zSSD fusion protein leads to stronger activation of the transduced T cell compared to the VL1VH3-CD28ATD-CD137CSD-CD3zSSD suggests that fusion of the VL domain to the anchoring domain (through a peptide linker) leads to a more potent antigen binding receptor. This is unexpected and surprising.

Combination of the VH domain VH3 with the VL domain VL1, both identified by the present inventors is especially favorable since these variable domains are humanized antibody domains. Without being bound to theory humanized antibody domains are preferable since less side effect can be expected when applying antigen binding moieties comprising such humanized antibody domains to human patients (such as e.g. less formation of anti-drug antibodies (ADA)). However, humanization can result in loss of binding of an antigen binding moiety (e.g. one deriving from a non-human source). As shown in the appended Examples, the humanized VH3 and VL1 domains retain binding to an Fc domain comprising the comprising the amino acid mutation P329G according to EU numbering. This result is unexpected as shown for example by the failure of other humanized VH and VL domains to retain comparable binding to an Fc domain comprising the amino acid mutation P329G according to EU numbering.

Hence, in a preferred embodiment of the present invention the heterodimeric antibody is combined with an antigen binding receptor comprising a humanized antigen binding moiety.

Pairing of a tumor-specific antibody, i.e. a antibody, comprising a heterodimeric Fc domain (e.g. comprising the amino acid mutation P329G according to EU numbering with T cells transduced with an antigen binding receptor which comprise/consist of an extracellular domain comprising an antigen binding moiety capable of specific binding to the mutated Fc domain results in a specific activation of the T cells and subsequent lysis of the tumor cell. This approach bears significant safety advantages over conventional T cell based approaches, as the T cell would be inert in the absence of the antibody comprising the mutated Fc domain. Accordingly, the invention provides a versatile therapeutic platform wherein IgG type antibodies are used to mark or label tumor cells as a guidance for T cell and wherein transduced T cells are specifically targeted toward the tumor cells by providing specificity for a mutated Fc domain of the IgG type antibody. After binding to the mutated Fc domain of the antibody on the surface of a tumor cell, the transduced T cell as described herein becomes activated and the tumor cell will subsequently be lysed.

### Antigen Binding Moieties for Antigen Binding Receptors

In an illustrative embodiment of the present invention, as a proof of concept, provided are humanized antigen binding receptors capable of specific binding to a mutated Fc domain comprising the amino acid mutation P329G and effector cells expressing said antigen binding receptors. The P329G mutation reduces binding to Fcγ receptors and associated effector function. Accordingly, the mutated Fc domain comprising the P329G mutation binds to Fcγ receptors with reduced or abolished affinity compared to the non-mutated Fc domain.

In one embodiment the antigen binding moiety is capable of specific binding to a mutated Fc domain composed of a first and a second subunit capable of stable association. In one embodiment the Fc domain is an IgG, specifically an IgG₁ domain. In one embodiment the Fc domain is a human Fc domain.

In a preferred embodiment, the Fc domain comprises the P329G mutation.

In one embodiment, the antigen binding receptor comprises an extracellular domain comprising an antigen binding moiety. In one embodiment, the antigen binding moiety is capable of specific binding to an Fc domain comprising the amino acid mutation P329G according to EU numbering

In one embodiment, the antigen binding moiety comprises a heavy chain variable domain (VH) comprising at least one of:
(a) a heavy chain complementarity determining region (CDR H) 1 amino acid sequence of RYWMN (SEQ ID NO:1);
(b) a CDR H2 amino acid sequence of EITPDSSTINYAPSLKG (SEQ ID NO:2) or of EITPDSSTINYTPSLKG (SEQ ID NO:40); and
(c) a CDR H3 amino acid sequence of PYDYGAWFAS (SEQ ID NO:3).

In one embodiment the antigen binding moiety comprises a light chain variable domain (VL) comprising at least one of:
(d) a light chain (CDR L)1 amino acid sequence of RSSTGAVTTSNYAN (SEQ ID NO:4);
(e) a CDR L2 amino acid sequence of GTNKRAP (SEQ ID NO:5); and
(f) a CDR L3 amino acid sequence of ALWYSNHWV (SEQ ID NO:6).

In a preferred embodiment the antigen binding moiety comprises a heavy chain variable domain (VH) comprising:
(a) a heavy chain complementarity determining region (CDR H) 1 amino acid sequence of RYWMN (SEQ ID NO:1);
(b) a CDR H2 amino acid sequence of EITPDSSTINYAPSLKG (SEQ ID NO:2) or of EITPDSSTINYTPSLKG (SEQ ID NO:40);
(c) a CDR H3 amino acid sequence of PYDYGAWFAS (SEQ ID NO:3); and a light chain variable domain (VL) comprising:
(d) a light chain (CDR L)1 amino acid sequence of RSSTGAVTTSNYAN (SEQ ID NO:4);
(e) a CDR L2 amino acid sequence of GTNKRAP (SEQ ID NO:5); and
(f) a CDR L3 amino acid sequence of ALWYSNHWV (SEQ ID NO:6).

In a preferred embodiment the antigen binding moiety comprises a heavy chain variable domain (VH) comprising:
(a) a heavy chain complementarity determining region (CDR H) 1 amino acid sequence of RYWMN (SEQ ID NO:1);
(b) a CDR H2 amino acid sequence of EITPDSSTINYAPSLKG (SEQ ID NO:2);
(c) a CDR H3 amino acid sequence of PYDYGAWFAS (SEQ ID NO:3); and a light chain variable domain (VL) comprising:
(d) a light chain (CDR L)1 amino acid sequence of RSSTGAVTTSNYAN (SEQ ID NO:4);
(e) a CDR L2 amino acid sequence of GTNKRAP (SEQ ID NO:5); and
(f) a CDR L3 amino acid sequence of ALWYSNHWV (SEQ ID NO:6).

In another particular embodiment the antigen binding moiety comprises a heavy chain variable domain (VH) comprising:
(a) a heavy chain complementarity determining region (CDR H) 1 amino acid sequence of RYWMN (SEQ ID NO:1);
(b) a CDR H2 amino acid sequence of EITPDSSTINYTPSLKG (SEQ ID NO:40);
(c) a CDR H3 amino acid sequence of PYDYGAWFAS (SEQ ID NO:3); and a light chain variable domain (VL) comprising:
(d) a light chain (CDR L)1 amino acid sequence of RSSTGAVTTSNYAN (SEQ ID NO:4);
(e) a CDR L2 amino acid sequence of GTNKRAP (SEQ ID NO:5); and
(f) a CDR L3 amino acid sequence of ALWYSNHWV (SEQ ID NO:6).

In one embodiment the antigen binding moiety comprises a heavy chain variable domain (VH) comprising an amino acid sequence that is at least about 95%, 96%, 97%, 98%, 99% or 100% identical to an amino acid sequence selected from the group consisting of SEQ ID NO:8, SEQ ID NO:41 and SEQ ID NO:44.

In one embodiment the antigen binding moiety comprises a heavy chain variable domain (VH) comprising an amino acid sequence that is at least about 95%, 96%, 97%, 98%, 99% or 100% identical to the amino acid sequence of SEQ ID NO:8.

In one embodiment the antigen binding moiety comprises a heavy chain variable domain (VH) comprising an amino acid sequence that is at least about 95%, 96%, 97%, 98%, 99% or 100% identical to the amino acid sequence of SEQ ID NO:41.

In one embodiment the antigen binding moiety comprises a heavy chain variable domain (VH) comprising an amino acid sequence that is at least about 95%, 96%, 97%, 98%, 99% or 100% identical to the amino acid sequence of SEQ ID NO:44.

In one embodiment the antigen binding moiety comprises a light chain variable domain (VL) comprising an amino acid sequence that is at least about 95%, 96%, 97%, 98%, 99% or 100% identical to the amino acid sequence of SEQ ID NO:9.

In one embodiment, the antigen binding moiety comprises a heavy chain variable domain (VH) comprising an amino acid sequence that is at least about 95%, 96%, 97%, 98%, 99% or 100% identical to the amino acid sequence of SEQ ID NO:8 and a light chain variable domain (VL) comprising an amino acid sequence that is at least about 95%, 96%, 97%, 98%, 99% or 100% identical to the amino acid sequence of SEQ ID NO:9.

In one embodiment, the antigen binding moiety comprises a heavy chain variable domain (VH) comprising an amino acid sequence that is at least about 95%, 96%, 97%, 98%, 99% or 100% identical to the amino acid sequence of SEQ ID NO:41 and a light chain variable domain (VL) comprising an amino acid sequence that is at least about 95%, 96%, 97%, 98%, 99% or 100% identical to the amino acid sequence of SEQ ID NO:9.

In one embodiment, the antigen binding moiety comprises a heavy chain variable domain (VH) comprising an amino acid sequence that is at least about 95%, 96%, 97%, 98%, 99% or 100% identical to the amino acid sequence of SEQ ID NO:44 and a light chain variable domain (VL) comprising an amino acid sequence that is at least about 95%, 96%, 97%, 98%, 99% or 100% identical to the amino acid sequence of SEQ ID NO:9.

In a preferred embodiment the antigen binding moiety comprises a heavy chain variable domain (VH) comprising the amino acid sequence of SEQ ID NO: 8, and a light chain variable domain (VL) comprising the amino acid sequence of SEQ ID NO:9.

In one embodiment, the antigen binding moiety is a scFv, or a scFab. In a preferred embodiment, the antigen binding moiety is a scFv.

In one embodiment, the antigen binding moiety comprises a heavy chain variable domain (VH) and a light chain variable domain (VL), wherein the VH domain is connected to the VL domain, in particular through a peptide linker. In one embodiment, the C-terminus of the VL domain is connected to the N-terminus of the VH domain, in particular through a peptide linker. In a preferred embodiment, the C-terminus of the VH domain is connected to the N-terminus of the VL domain, in particular through a peptide linker. In one embodiment, the peptide linker comprises the amino acid sequence GGGGSGGGGSGGGGSGGGGS (SEQ ID NO:16).

In one embodiment the antigen binding moiety is a scFv which is a polypeptide consisting of an heavy chain variable domain (VH), an light chain variable domain (VL) and a linker, wherein said variable domains and said linker have one of the following configurations in N-terminal to C-terminal direction: a) VH-linker-VL or b) VL-linker-VH. In a preferred embodiment, the scFv has the configuration VH-linker-VL.

In one embodiment, the antigen binding moiety comprises an amino acid sequence that is at least about 95%, 96%, 97%, 98%, 99% or 100% identical to an amino acid sequence selected from the group consisting of SEQ ID NO:10, SEQ ID NO:126 and SEQ ID NO:128.

In one embodiment, the antigen binding moiety comprises an amino acid sequence that is at least about 95%, 96%, 97%, 98%, 99% or 100% identical to the amino acid sequence of SEQ ID NO:10. In one embodiment, the antigen binding moiety comprises the amino acid sequence of SEQ ID NO: 10.

In one embodiment, the antigen binding moiety comprises an amino acid sequence that is at least about 95%, 96%, 97%, 98%, 99% or 100% identical to the amino acid sequence of SEQ ID NO:126. In one embodiment, the antigen binding moiety comprises the amino acid sequence of SEQ ID NO:126.

In one embodiment, the antigen binding moiety comprises an amino acid sequence that is at least about 95%, 96%, 97%, 98%, 99% or 100% identical to the amino acid sequence of SEQ ID NO:128. In one embodiment, the antigen binding moiety comprises the amino acid sequence of SEQ ID NO:128.

Antigen binding moieties comprising a heavy chain variable domain (VH) and a light chain variable domain (VL), such as the scFv and scFab fragments as described herein may be further stabilized by introducing interchain disulfide bridges between the VH and the VL domain. Accordingly, in one embodiment, the scFv fragment(s) and/or the scFab fragment(s) comprised in the antigen binding receptors according to the invention are further stabilized by generation of interchain disulfide bonds via insertion of cysteine residues (e.g., position 44 in the variable heavy chain and position 100 in the variable light chain according to Kabat numbering). In one embodiment, provided is any one of the above provided VH and/or VL sequences comprising at least one substitution of an amino acid with cysteine (in particular at position 44 in the variable heavy chain and/or position 100 in the variable light chain according to Kabat numbering).

### Anchoring Transmembrane Domain (ATD)

In the context of the present invention, the anchoring transmembrane domain of the antigen binding receptors may be characterized by not having a cleavage site for mammalian proteases. In the context of the present invention, proteases refer to proteolytic enzymes that are able to hydrolyze the amino acid sequence of a transmembrane domain comprising a cleavage site for the protease. The term proteases include both endopeptidases and exopeptidases. In the context of the present invention any anchoring transmembrane domain of a transmembrane protein as laid down among others by the CD-nomenclature may be used to generate the antigen binding receptors of the invention.

Accordingly, in the context of the present invention, the anchoring transmembrane domain may comprise part of a murine/mouse or preferably of a human transmembrane domain. An example for such an anchoring transmembrane domain is a transmembrane domain of CD8, for example, having the amino acid sequence as shown herein in SEQ ID NO:11 (as encoded by the DNA sequence shown in SEQ ID NO:24). In the context of the present invention, the anchoring transmembrane domain of the antigen binding receptor of the present invention may comprise/consist of an amino acid sequence as shown in SEQ ID NO:11 (as encoded by the DNA sequence shown in SEQ ID NO:24).

In another embodiment, the herein provided antigen binding receptor may comprise the transmembrane domain of CD28 which is located at amino acids 153 to 179, 154 to 179, 155 to 179, 156 to 179, 157 to 179, 158 to 179, 159 to 179, 160 to 179, 161 to 179, 162 to 179, 163 to 179, 164 to 179, 165 to 179, 166 to 179, 167 to 179, 168 to 179, 169 to 179, 170 to 179, 171 to 179, 172 to 179, 173 to 179, 174 to 179, 175 to 179, 176 to 179, 177 to 179 or 178 to 179 of the human full length CD28 protein as shown in SEQ ID NO:61 (as encoded by the cDNA shown in SEQ ID NO:70).

Alternatively, any protein having a transmembrane domain, as provided among others by the CD nomenclature, may be used as an anchoring transmembrane domain of the antigen binding receptor protein of the invention.

In some embodiments, the anchoring transmembrane domain comprises the transmembrane domain of any one of the group consisting of CD27 (SEQ ID NO:59 as encoded by SEQ ID NO:58), CD137 (SEQ ID NO:67 as encoded by SEQ ID NO:66), OX40 (SEQ ID NO:71, as encoded by SEQ ID NO:70), ICOS (SEQ ID NO:75 as encoded by SEQ ID NO:74), DAP10 (SEQ ID NO:80 as encoded by SEQ ID NO:79), DAP12 (SEQ ID NO:83 as encoded by SEQ ID NO:82), CD3z (SEQ ID NO:88 as encoded by SEQ ID NO:87), FCGR3A (SEQ ID NO:90 as encoded by SEQ ID NO:91), NKG2D (SEQ ID NO:94 as encoded by SEQ ID NO:95), CD8 (SEQ ID NO:123 as encoded by SEQ ID NO: 124), or a fragment of the transmembrane thereof that retains the capability to anchor the antigen binding receptor to the membrane.

Human sequences might be beneficial in the context of the common invention, for example because (parts) of the anchoring transmembrane domain might be accessible from the extracellular space and hence to the immune system of a patient. In a preferred embodiment, the anchoring transmembrane domain comprises a human sequence. In such embodiments, the anchoring transmembrane domain comprises the transmembrane domain of any one of the group consisting of human CD27 (SEQ ID NO:57 as encoded by SEQ ID NO:56), human CD137 (SEQ ID NO:65 as encoded by SEQ ID NO:64), human OX40 (SEQ ID NO:69, as encoded by SEQ ID NO:68), human ICOS (SEQ ID NO:73 as encoded by SEQ ID NO:72), human DAP10 (SEQ ID NO:78 as encoded by SEQ ID NO:77), human DAP12 (SEQ ID NO:81 as encoded by SEQ ID NO:80), human CD3z (SEQ ID NO:86 as encoded by SEQ ID NO:85), human FCGR3A (SEQ ID NO:88 as encoded by SEQ ID NO:89), human NKG2D (SEQ ID NO:92 as encoded by SEQ ID NO:93), human CD8 (SEQ ID NO:121 as encoded by SEQ ID NO:122), or a fragment of the transmembrane thereof that retains the capability to anchor the antigen binding receptor to the membrane.

### Stimulatory Signaling Domain (SSD) and Co-Stimulatory Signaling Domain (CSD)

Preferably, the antigen binding receptor comprises at least one stimulatory signaling domain and/or at least one co-stimulatory signaling domain. Accordingly, the herein provided antigen binding receptor preferably comprises a stimulatory signaling domain, which provides T cell activation. The herein provided antigen binding receptor may comprise a stimulatory signaling domain which is a fragment/polypeptide part of murine/mouse or human CD3z (the UniProt Entry of the human CD3z is P20963 (version number 177 with sequence number 2; the UniProt Entry of the murine/mouse CD3z is P24161 (primary citable accession number) or Q9D3G3 (secondary citable accession number) with the version number 143 and the sequence number 1)), FCGR3A (the UniProt Entry of the human FCGR3A is P08637 (version number 178 with sequence number 2)), or NKG2D (the UniProt Entry of the human NKG2D is P26718 (version number 151 with sequence number 1); the UniProt Entry of the murine/mouse NKG2D is 054709 (version number 132 with sequence number 2)).

Thus, the stimulatory signaling domain which is comprised in the herein provided antigen binding receptor may be a fragment/polypeptide part of the full length of CD3z, FCGR3A or NKG2D. The amino acid sequences of the murine/mouse full length of CD3z, or NKG2D are shown herein as SEQ ID NOs: 86 (CD3z), 90 (FCGR3A) or 94 (NKG2D) (murine/mouse as encoded by the DNA sequences shown in SEQ ID NOs:87 (CD3z), 91 (FCGR3A) or 95 (NKG2D). The amino acid sequences of the human full length CD3z, FCGR3A or NKG2D are shown herein as SEQ ID NOs:84 (CD3z), 88 (FCGR3A) or 92 (NKG2D) (human as encoded by the DNA sequences shown in SEQ ID NOs:85 (CD3z), 89 (FCGR3A) or 93 (NKG2D)). The antigen binding receptor of the present invention may comprise fragments of CD3z, FCGR3A or NKG2D as stimulatory domain, provided that at least one signaling domain is comprised. In particular, any part/fragment of CD3z, FCGR3A, or NKG2D is suitable as stimulatory domain as long as at least one signaling motive is comprised. However, more preferably, the antigen binding receptor of the present invention comprises polypeptides which are derived from human origin. Thus, more preferably, the herein provided antigen binding receptor comprises the amino acid sequences as shown herein as SEQ ID NOs:84 (CD3z), 88 (FCGR3A) or 92 (NKG2D) (human as encoded by the DNA sequences shown in SEQ ID NOs:85 (CD3z), 89 (FCGR3A) or 93 (NKG2D)). In one embodiment, the antigen binding receptor of the present invention may comprise or consist of the amino acid sequence shown in SEQ ID NO:13 (as encoded by the DNA sequence shown in SEQ ID NO:26). In further embodiments the antigen binding receptor comprises the sequence as shown in SEQ ID NO:13 or a sequence which has up to 1, 2, 3, 4, 5, 6, 7, 8, 9, 10, 11, 12, 13, 14, 15, 16, 17, 18, 19, 20, 21, 22, 23, 23, 24, 25, 26, 27, 28, 29 or 30 substitutions, deletions or insertions in comparison to SEQ ID NO:13 and which is characterized by having a stimulatory signaling activity. Specific configurations of antigen binding receptors comprising a stimulatory signaling domain (SSD) are provided herein below and in the Examples and Figures. The stimulatory signaling activity can be determined; e.g., by enhanced cytokine release, as measured by ELISA (IL-2, IFNγ, TNFα), enhanced proliferative activity (as measured by enhanced cell numbers), or enhanced lytic activity as measured by LDH release assays.

Furthermore, the herein provided antigen binding receptor preferably comprises at least one co-stimulatory signaling domain which provides additional activity to the T cell. The herein provided antigen binding receptor may comprise a co-stimulatory signaling domain which is a fragment/polypeptide part of murine/mouse or human CD28 (the UniProt Entry of the human CD28 is P10747 (version number 173 with sequence number 1); the UniProt Entry of the murine/mouse CD28 is P31041 (version number 134 with sequence number 2)), CD137 (the UniProt Entry of the human CD137 is Q07011 (version number 145 with sequence number 1); the UniProt Entry of murine/mouse CD137 is P20334 (version number 139 with sequence number 1)), OX40 (the UniProt Entry of the human OX40 is P23510 (version number 138 with sequence number 1); the UniProt Entry of murine/mouse OX40 is P43488 (version number 119 with sequence number 1)), ICOS (the UniProt Entry of the human ICOS is Q9Y6W8 (version number 126 with sequence number 1)); the UniProt Entry of the murine/mouse ICOS is Q9WV40 (primary citable accession number) or Q9JL17 (secondary citable accession number) with the version number 102 and sequence version 2)), CD27 (the UniProt Entry of the human CD27 is P26842 (version number 160 with sequence number 2); the Uniprot Entry of the murine/mouse CD27 is P41272 (version number 137 with sequence version 1)), 4-1-BB (the UniProt Entry of the murine/mouse 4-1-BB is P20334 (version number 140 with sequence version 1); the UniProt Entry of the human 4-1-BB is Q07011 (version number 146 with sequence version)), DAP10 (the UniProt Entry of the human DAP10 is Q9UBJ5 (version number 25 with sequence number 1); the UniProt entry of the murine/mouse DAP10 is Q9QUJ0 (primary citable accession number) or Q9R1E7 (secondary citable accession number) with the version number 101 and the sequence number 1)) or DAP12 (the UniProt Entry of the human DAP12 is 043914 (version number 146 and the sequence number 1); the UniProt entry of the murine/mouse DAP12 is 0054885 (primary citable accession number) or Q9R1E7 (secondary citable accession number) with the version number 123 and the sequence number 1). In certain embodiments of the present invention the antigen binding receptor of the present invention may comprise one or more, i.e. 1, 2, 3, 4, 5, 6 or 7 of the herein defined co-stimulatory signaling domains. Accordingly, in the context of the present invention, the antigen binding receptor of the present invention may comprise a fragment/polypeptide part of a murine/mouse or preferably of a human CD137 as first co-stimulatory signaling domain and the second co-stimulatory signaling domain is selected from the group consisting of the murine/mouse or preferably of the human CD27, CD28, CD137, OX40, ICOS, DAP10 and DAP12, or fragments thereof. Preferably, the antigen binding receptor of the present invention comprises a co-stimulatory signaling domain which is derived from a human origin. Thus, more preferably, the co-stimulatory signaling domain(s) which is (are) comprised in the antigen binding receptor of the present invention may comprise or consist of the amino acid sequence as shown in SEQ ID NO:12 (as encoded by the DNA sequence shown in SEQ ID NO:25).

Thus, the co-stimulatory signaling domain which may be optionally comprised in the herein provided antigen binding receptor is a fragment/polypeptide part of the full length CD27, CD28, CD137, OX40, ICOS, DAP10 or DAP12. The amino acid sequences of the murine/mouse full length CD27, CD28, CD137, OX40, ICOS, CD27, DAP10 and DAP12 are shown herein as SEQ ID NOs:59 (CD27), 63 (CD28), 67 (CD137), 71 (OX40), 75 (ICOS), 79 (DAP10) or 83 (DAP12) (murine/mouse as encoded by the DNA sequences shown in SEQ ID NOs:58 (CD27), 62 (CD28), 66 (CD137), 70 (OX40), 74 (ICOS), 78 (DAP10) or 82 (DAP12)). However, because human sequences are most preferred in the context of the present invention, the co-stimulatory signaling domain which may be optionally comprised in the herein provided antigen binding receptor protein is a fragment/polypeptide part of the human full length CD27, CD28, CD137, OX40, ICOS, DAP10 or DAP12. The amino acid sequences of the human full length CD27, CD28, CD137, OX40, ICOS, DAP10 or DAP12 are shown herein as SEQ ID NOs: 57, (CD27), 61 (CD28), 65 (CD137), 69 (OX40), 73 (ICOS), 77 (DAP10) or 81 (DAP12) (human as encoded by the DNA sequences shown in SEQ ID NOs: 56 (CD27), 60 (CD28), 64 (CD137), 68 (OX40), 72 (ICOS), 76 (DAP10) or 80 (DAP12)).

In one preferred embodiment, the antigen binding receptor comprises CD28 or a fragment thereof as co-stimulatory signaling domain. The herein provided antigen binding receptor may comprise a fragment of CD28 as co-stimulatory signaling domain, provided that at least one signaling domain of CD28 is comprised. In particular, any part/fragment of CD28 is suitable for the antigen binding receptor of the invention as long as at least one of the signaling motives of CD28 is comprised. The co-stimulatory signaling domains PYAP (AA 208 to 211 of CD28) and YMNM (AA 191 to 194 of CD28) are beneficial for the function of the CD28 polypeptide and the functional effects enumerated above. The amino acid sequence of the YMNM domain is shown in SEQ ID NO:96; the amino acid sequence of the PYAP domain is shown in SEQ ID NO:97. Accordingly, in the antigen binding receptor of the present invention, the CD28 polypeptide preferably comprises a sequence derived from intracellular domain of a CD28 polypeptide having the sequences YMNM (SEQ ID NO:96) and/or PYAP (SEQ ID NO:97). In other embodiments, in the antigen binding receptor of the present invention, one or both of these domains are mutated to FMNM (SEQ ID NO:98) and/or AYAA (SEQ ID NO:99), respectively. Either of these mutations reduces the ability of a transduced cell comprising the antigen binding receptor to release cytokines without affecting its ability to proliferate and can advantageously be used to prolong the viability and thus the therapeutic potential of the transduced cells. Or, in other words, such a non-functional mutation preferably enhances the persistence of the cells which are transduced with the herein provided antigen binding receptor in vivo. These signaling motives may, however, be present at any site within the intracellular domain of the herein provided antigen binding receptor.

In another preferred embodiment, the antigen binding receptor comprises CD137 or a fragment thereof as co-stimulatory signaling domain. The herein provided antigen binding receptor may comprise a fragment of CD137 as co-stimulatory signaling domain, provided that at least one signaling domain of CD137 is comprised. In particular, any part/fragment of CD137 is suitable for the antigen binding receptor of the invention as long as at least one of the signaling motives of CD137 is comprised. In a preferred embodiment, the CD137 polypeptide which is comprised in the antigen binding receptor protein of the present invention comprises or consists of the amino acid sequence shown in SEQ ID NO:12 (as encoded by the DNA sequence shown in SEQ ID NO:25).

Specific configurations of antigen binding receptors comprising a co-stimulatory signaling domain (CSD) are provided herein below and in the Examples and Figures. The co-stimulatory signaling activity can be determined; e.g., by enhanced cytokine release, as measured by ELISA (IL-2, IFNγ, TNFα), enhanced proliferative activity (as measured by enhanced cell numbers), or enhanced lytic activity as measured by LDH release assays. As mentioned above, in an embodiment of the present invention, the co-stimulatory signaling domain of the antigen binding receptor may be derived from the human CD28 and/or CD137 gene T cell activity, defined as cytokine production, proliferation and lytic activity of the transduced cell described herein, like a transduced T cell. CD28 and/or CD137 activity can be measured by release of cytokines by ELISA or flow cytometry of cytokines such as interferon-gamma (IFN-D Q or interleukin 2 (IL-2), proliferation of T cells measured e.g. by ki67-measurement, cell quantification by flow cytometry, or lytic activity as assessed by real time impedance measurement of the target cell (by using e.g. an ICELLligence instrument as described e.g. in Thakur et al., Biosens Bioelectron. 35(1) (2012), 503-506; Krutzik et al., Methods Mol Biol. 699 (2011), 179-202; Ekkens et al., Infect Immun. 75(5) (2007), 2291-2296; Ge et al., Proc Natl Acad Sci USA. 99(5) (2002), 2983-2988; Duwell et al., Cell Death Differ. 21(12) (2014), 1825-1837, Erratum in: Cell Death Differ. 21(12) (2014), 161).

### Linker and Signal Peptides

Moreover, the herein provided antigen binding receptor may comprise at least one linker (or “spacer”). A linker is usually a peptide having a length of up to 20 amino acids. Accordingly, in the context of the present invention the linker may have a length of 1, 2, 3, 4, 5, 6, 7, 8, 9, 10, 11, 12, 13, 14, 15, 16, 17, 18, 19 or 20 amino acids. For example, the herein provided antigen binding receptor may comprise a linker between the extracellular domain comprising at least one antigen binding moiety capable of specific binding to a mutated Fc domain, the anchoring transmembrane domain, the co-stimulatory signaling domain and/or the stimulatory signaling domain. Furthermore, the herein provided antigen binding receptor may comprise a linker in the antigen binding moiety, in particular between immunoglobulin domains of the antigen binding moiety (such as between VH and VL domains of a scFv). Such linkers have the advantage that they increase the probability that the different polypeptides of the antigen binding receptor (i.e. the extracellular domain comprising at least one antigen binding moiety, the anchoring transmembrane domain, the co-stimulatory signaling domain and/or the stimulatory signaling domain) fold independently and behave as expected. Thus, in the context of the present invention, the extracellular domain comprising at least one antigen binding moiety, the anchoring transmembrane domain, the co-stimulatory signaling domain and the stimulatory signaling domain may be comprised in a single-chain multi-functional polypeptide. A single-chain fusion construct e.g. may consist of (a) polypeptide(s) comprising (an) extracellular domain(s) comprising at least one antigen binding moiety, (an) anchoring transmembrane domain(s), (a) co-stimulatory signaling domain(s) and/or (a) stimulatory signaling domain(s). Accordingly, the antigen binding moiety, the anchoring transmembrane domain, the co-stimulatory signaling domain and the stimulatory signaling domain may be connected by one or more identical or different peptide linker as described herein. For example, in the herein provided antigen binding receptor the linker between the extracellular domain comprising at least one antigen binding moiety and the anchoring transmembrane domain may comprise or consist of the amino and amino acid sequence as shown in SEQ ID NO:17. In another embodiment, the linker between the antigen binding moiety and the anchoring transmembrane domain comprises or consists of the amino and amino acid sequence as shown in SEQ ID NO:19. Accordingly, the anchoring transmembrane domain, the co-stimulatory signaling domain and/or the stimulatory domain may be connected to each other by peptide linkers or alternatively, by direct fusion of the domains.

In preferred embodiments according to the invention the antigen binding moiety comprised in the extracellular domain is a single-chain variable fragment (scFv) which is a fusion protein of the variable domains of the heavy (VH) and light chains (VL) of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. In a preferred embodiment, the linker connects the N-terminus of the VL domain with the C-terminus of the VH domain. For example, in the herein provided antigen binding receptor the linker may have the amino and amino acid sequence as shown in SEQ ID NO:16. scFv antibodies are, e.g. described in Houston, J. S., Methods in Enzymol. 203 (1991) 46-96).

In some embodiments according to the invention the antigen binding moiety comprised in the extracellular domain is a single chain Fab fragment or scFab which is a polypeptide consisting of an heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain.

The herein provided antigen binding receptor or parts thereof may comprise a signal peptide. Such a signal peptide will bring the protein to the surface of the T cell membrane. For example, in the herein provided antigen binding receptor the signal peptide may have the amino and amino acid sequence as shown in SEQ ID NO:100 (as encoded by the DNA sequence shown in SEQ ID NO:101).

### Specific Configurations of Antigen Binding Receptors

The components of the antigen binding receptors as described herein can be fused to each other in a variety of configurations to generate T cell activating antigen binding receptors.

In some embodiments, the antigen binding receptor comprises an extracellular domain composed of a heavy chain variable domain (VH) and a light chain variable domain (VL) connected to an anchoring transmembrane domain. In preferred embodiments, the VH domain is fused at the C-terminus to the N-terminus of the VL domain, optionally through a peptide linker. In other embodiments, the antigen binding receptor further comprises a stimulatory signaling domain and/or a co-stimulatory signaling domain. In a specific such embodiment, the antigen binding receptor essentially consists of a VH domain and a VL domain, an anchoring transmembrane domain, and optionally a stimulatory signaling domain connected by one or more peptide linkers, wherein the VH domain is fused at the C-terminus to the N-terminus of the VL domain, and the VL domain is fused at the C-terminus to the N-terminus of the anchoring transmembrane domain, wherein the anchoring transmembrane domain is fused at the C-terminus to the N-terminus of the stimulatory signaling domain. Optionally, the antigen binding receptor further comprises a co-stimulatory signaling domain. In one such specific embodiment, the antigen binding receptor essentially consists of a VH domain and a VL domain, an anchoring transmembrane domain, a stimulatory signaling domain and a co-stimulatory signaling domain connected by one or more peptide linkers, wherein the VH domain is fused at the C-terminus to the N-terminus of the VL domain, and the VL domain is fused at the C-terminus to the N-terminus of the anchoring transmembrane domain, wherein the anchoring transmembrane domain is fused at the C-terminus to the N-terminus of the stimulatory signaling domain, wherein the stimulatory signaling domain is fused at the C-terminus to the N-terminus of the co-stimulatory signaling domain. In an alternative embodiment, the co-stimulatory signaling domain is connected to the anchoring transmembrane domain instead of the stimulatory signaling domain. In a preferred embodiment, the antigen binding receptor essentially consists of a VH domain and a VL domain, an anchoring transmembrane domain, a co-stimulatory signaling domain and a stimulatory signaling domain connected by one or more peptide linkers, wherein the VH domain is fused at the C-terminus to the N-terminus of the VL domain, and the VL domain is fused at the C-terminus to the N-terminus of the anchoring transmembrane domain, wherein the anchoring transmembrane domain is fused at the C-terminus to the N-terminus of the co-stimulatory signaling domain, wherein the co-stimulatory signaling domain is fused at the C-terminus to the N-terminus of the stimulatory signaling domain.

The antigen binding moiety, the anchoring transmembrane domain and the stimulatory signaling and/or co-stimulatory signaling domains may be fused to each other directly or through one or more peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G4S)₁ or G4(SG4)ₙ peptide linkers, wherein “n” is generally a number between 1 and 10, typically between 2 and 4. A preferred peptide linker for connecting the antigen binding moiety and the anchoring transmembrane moiety is GGGGS (G₄S) according to SEQ ID NO 17. Another preferred peptide linker for connecting the antigen binding moiety and the anchoring transmembrane moiety is KPTTTPAPRPPTPAPTIASQPLSLRPEACRPAAGGAVHTRGLDFACD (CD8stalk) according to SEQ ID NO 19. An exemplary peptide linker suitable for connecting variable heavy chain domain (VH) and the variable light chain domain (VL) is GGGSGGGSGGGSGGGS (G4S)4 according to SEQ ID NO 16.

Additionally, linkers may comprise (a portion of) an immunoglobulin hinge region. Particularly where an antigen binding moiety is fused to the N-terminus of an anchoring transmembrane domain, it may be fused via an immunoglobulin hinge region or a portion thereof, with or without an additional peptide linker.

As described herein, the antigen binding receptors of the present invention comprise an extracellular domain comprising at least one antigen binding moiety. An antigen binding receptor with a single antigen binding moiety capable of specific binding to a target cell antigen is useful and preferred, particularly in cases where high expression of the antigen binding receptor is needed. In such cases, the presence of more than one antigen binding moiety specific for the target cell antigen may limit the expression efficiency of the antigen binding receptor. In other cases, however, it will be advantageous to have an antigen binding receptor comprising two or more antigen binding moieties specific for a target cell antigen, for example to optimize targeting to the target site or to allow crosslinking of target cell antigens.

In one particular embodiment, the antigen binding receptor comprises one antigen binding moiety capable of specific binding to a mutated Fc domain, in particular an IgG1 Fc domain, comprising the P329G mutation (according to EU numbering). In one embodiment, the antigen binding moiety capable of specific binding to a mutated Fc domain but not capable of specific binding to the non-mutated parent Fc domain is a scFv.

In one embodiment, the antigen binding moiety is fused at the C-terminus of the scFv fragment to the N-terminus of an anchoring transmembrane domain, optionally through a peptide linker. In one embodiment the peptide linker comprises the amino acid sequence KPTTTPAPRPPTPAPTIASQPLSLRPEACRPAAGGAVHTRGLDFACD (SEQ ID NO:19). In one embodiment, the anchoring transmembrane domain is a transmembrane domain selected from the group consisting of the CD8, the CD4, the CD3z, the FCGR3A, the NKG2D, the CD27, the CD28, the CD137, the OX40, the ICOS, the DAP10 or the DAP12 transmembrane domain or a fragment thereof. In a preferred embodiment, the anchoring transmembrane domain is the CD8 transmembrane domain or a fragment thereof. In a particular embodiment, the anchoring transmembrane domain comprises or consist of the amino acid sequence of IYIWAPLAGTCGVLLLSLVIT (SEQ ID NO:11). In one embodiment, the antigen binding receptor further comprises a co-stimulatory signaling domain (CSD). In one embodiment, the anchoring transmembrane domain of the antigen binding receptor is fused at the C-terminus to the N-terminus of a co-stimulatory signaling domain. In one embodiment, the co-stimulatory signaling domain is individually selected from the group consisting of the intracellular domain of CD27, of CD28, of CD137, of OX40, of ICOS, of DAP10 and of DAP12, or fragments thereof as described herein before. In a preferred embodiment, the co-stimulatory signaling domain is the intracellular domain of CD28 or a fragment thereof. In one preferred embodiment, the co-stimulatory signaling domain comprises the intracellular domain of CD28 or a fragment thereof that retains CD28 signaling. In another preferred embodiment, the co-stimulatory signaling domain comprises the intracellular domain of CD137 or a fragment thereof that retains CD137 signaling. In a particular embodiment the co-stimulatory signaling domain comprises or consists of SEQ ID NO:12. In one embodiment, the antigen binding receptor further comprises a stimulatory signaling domain. In one embodiment, the co-stimulatory signaling domain of the antigen binding receptor is fused at the C-terminus to the N-terminus of the stimulatory signaling domain. In one embodiment, the at least one stimulatory signaling domain is individually selected from the group consisting of the intracellular domain of CD3z, FCGR3A and NKG2D, or fragments thereof. In a preferred embodiment, the co-stimulatory signaling domain is the intracellular domain of CD3z or a fragment thereof that retains CD3z signaling. In a particular embodiment the co-stimulatory signaling domain comprises or consists of SEQ ID NO:13.

In one embodiment, the antigen binding receptor is fused to a reporter protein, particularly to GFP or enhanced analogs thereof. In one embodiment, the antigen binding receptor is fused at the C-terminus to the N-terminus of eGFP (enhanced green fluorescent protein), optionally through a peptide linker as described herein. In a preferred embodiment, the peptide linker is GEGRGSLLTCGDVEENPGP (T2A) according to SEQ ID NO:18.

In a particular embodiment, the antigen binding receptor comprises an anchoring transmembrane domain and an extracellular domain comprising at least one antigen binding moiety, wherein the at least one antigen binding moiety is a scFv capable of specific binding to a mutated Fc domain but not capable of specific binding to the non-mutated parent Fc domain, wherein the mutated Fc domain comprises the P329G mutation (according to EU numbering). The P329G mutation reduces Fcγ receptor binding. In one embodiment, the antigen binding receptor of the invention comprises an anchoring transmembrane domain (ATD), a co-stimulatory signaling domain (CSD) and a stimulatory signaling domain (SSD). In one such embodiment, the antigen binding receptor has the configuration scFv-ATD-CSD-SSD. In a preferred embodiment, the antigen binding receptor has the configuration VH-VL-ATD-CSD-SSD. In a more specific such embodiment, the antigen binding receptor has the configuration VH-linker-VL-linker-ATD-CSD-SSD.

In a particular embodiment, the antigen binding moiety is a scFv capable of specific binding to a mutated Fc domain comprising the P329G mutation, wherein the antigen binding moiety comprises at least one heavy chain complementarity determining region (CDR) selected from the group consisting of SEQ ID NO:1, SEQ ID NO:2 and SEQ ID NO:3 and at least one light chain CDR selected from the group of SEQ ID NO:4, SEQ ID NO:5, SEQ ID NO:6.

In another particular embodiment, the antigen binding moiety is a scFv capable of specific binding to a mutated Fc domain comprising the P329G mutation, wherein the antigen binding moiety comprises at least one heavy chain complementarity determining region (CDR) selected from the group consisting of SEQ ID NO:1, SEQ ID NO:40 and SEQ ID NO:3 and at least one light chain CDR selected from the group of SEQ ID NO:4, SEQ ID NO:5, SEQ ID NO:6.

In a preferred embodiment, the antigen binding moiety is a scFv capable of specific binding to a mutated Fc domain comprising the P329G mutation, wherein the antigen binding moiety comprises the complementarity determining region (CDR H) 1 amino acid sequence RYWMN (SEQ ID NO:1), the CDR H2 amino acid sequence EITPDSSTINYAPSLKG (SEQ ID NO:2), the CDR H3 amino acid sequence PYDYGAWFAS (SEQ ID NO:3), the light chain complementary-determining region (CDR L) 1 amino acid sequence RSSTGAVTTSNYAN (SEQ ID NO:4), the CDR L2 amino acid sequence GTNKRAP (SEQ ID NO:5) and the CDR L3 amino acid sequence ALWYSNHWV (SEQ ID NO:6).

In a preferred embodiment, the antigen binding receptor comprises in order from the N-terminus to the C-terminus:
(i) a heavy chain variable domain (VH) comprising the heavy chain complementarity determining region (CDR) 1 of SEQ ID NO:1, the heavy chain CDR 2 of SEQ ID NO:2, the heavy chain CDR 3 of SEQ ID NO:3,
(ii) a peptide linker, in particular the peptide linker of SEQ ID NO:16,
(iii) a light chain variable domain (VL) comprising the light chain CDR 1 of SEQ ID NO:4, the light chain CDR 2 of SEQ ID NO:5 and the light chain CDR 3 of SEQ ID NO:6,
(iv) a peptide linker, in particular the peptide linker of SEQ ID NO:19,
(v) an anchoring transmembrane domain, in particular the anchoring transmembrane domain of SEQ ID NO:11,
(vi) a co-stimulatory signaling domain, in particular the co-stimulatory signaling domain of SEQ ID NO:12, and
(vii) a stimulatory signaling domain, in particular the stimulatory signaling domain of SEQ ID NO:13.

In one embodiment, the antigen binding receptor comprises in order from the N-terminus to the C-terminus:
(i) a heavy chain variable domain (VH) comprising the heavy chain complementarity determining region (CDR) 1 of SEQ ID NO:1, the heavy chain CDR 2 of SEQ ID NO:40, the heavy chain CDR 3 of SEQ ID NO:3,
(ii) a peptide linker, in particular the peptide linker of SEQ ID NO:16,
(iii) a light chain variable domain (VL) comprising the light chain CDR 1 of SEQ ID NO:4, the light chain CDR 2 of SEQ ID NO:5 and the light chain CDR 3 of SEQ ID NO:6,
(iv) a peptide linker, in particular the peptide linker of SEQ ID NO:19,
(v) an anchoring transmembrane domain, in particular the anchoring transmembrane domain of SEQ ID NO:11,
(vi) a co-stimulatory signaling domain, in particular the co-stimulatory signaling domain of SEQ ID NO:12, and
(vii) a stimulatory signaling domain, in particular the stimulatory signaling domain of SEQ ID NO:13.

In one embodiment, the antigen binding receptor comprises in order from the N-terminus to the C-terminus
(i) a heavy chain variable domain (VH),
(ii) a peptide linker, in particular the peptide linker of SEQ ID NO:16,
(iii) a light chain variable domain (VL) that is at least about 95%, 96%, 97%, 98%, 99% or 100% identical to the amino acid sequence of SEQ ID NO:9, wherein the VH and VL domains are capable of forming an antigen binding moiety that binds to an Fc domain comprising the amino acid mutation P329G according to EU numbering,
(iv) a peptide linker, in particular the peptide linker of SEQ ID NO:19,
(v) an anchoring transmembrane domain, in particular an anchoring transmembrane domain that is at least about 95%, 96%, 97%, 98%, 99% or 100% identical to the amino acid sequence of SEQ ID NO:11,
(vi) a co-stimulatory signaling domain, in particular a co-stimulatory signaling domain that is at least about 95%, 96%, 97%, 98%, 99% or 100% identical to the amino acid sequence of SEQ ID NO:12, and
(vii) a stimulatory signaling domain, in particular a stimulatory signaling domain that is at least about 95%, 96%, 97%, 98%, 99% or 100% identical to the amino acid sequence of SEQ ID NO:13.

In one embodiment, the antigen binding receptor comprises in order from the N-terminus to the C-terminus
(i) a heavy chain variable domain (VH) that is at least about 95%, 96%, 97%, 98%, 99% or 100% identical to the amino acid sequence of SEQ ID NO:8,
(ii) a peptide linker, in particular the peptide linker of SEQ ID NO:16,
(iii) a light chain variable domain (VL) that is at least about 95%, 96%, 97%, 98%, 99% or 100% identical to the amino acid sequence of SEQ ID NO:9,
(iv) a peptide linker, in particular the peptide linker of SEQ ID NO:19,
(v) an anchoring transmembrane domain, in particular an anchoring transmembrane domain that is at least about 95%, 96%, 97%, 98%, 99% or 100% identical to the amino acid sequence of SEQ ID NO:11,
(vi) a co-stimulatory signaling domain, in particular a co-stimulatory signaling domain that is at least about 95%, 96%, 97%, 98%, 99% or 100% identical to the amino acid sequence of SEQ ID NO:12, and
(vii) a stimulatory signaling domain, in particular a stimulatory signaling domain that is at least about 95%, 96%, 97%, 98%, 99% or 100% identical to the amino acid sequence of SEQ ID NO:13.

In one embodiment, the antigen binding receptor comprises in order from the N-terminus to the C-terminus
(i) a heavy chain variable domain (VH) that is at least about 95%, 96%, 97%, 98%, 99% or 100% identical to the amino acid sequence of SEQ ID NO:41,
(ii) a peptide linker, in particular the peptide linker of SEQ ID NO:16,
(iii) a light chain variable domain (VL) that is at least about 95%, 96%, 97%, 98%, 99% or 100% identical to the amino acid sequence of SEQ ID NO:9,
(iv) a peptide linker, in particular the peptide linker of SEQ ID NO:19,
(v) an anchoring transmembrane domain, in particular an anchoring transmembrane domain that is at least about 95%, 96%, 97%, 98%, 99% or 100% identical to the amino acid sequence of SEQ ID NO:11,
(vi) a co-stimulatory signaling domain, in particular a co-stimulatory signaling domain that is at least about 95%, 96%, 97%, 98%, 99% or 100% identical to the amino acid sequence of SEQ ID NO:12, and
(vii) a stimulatory signaling domain, in particular a stimulatory signaling domain that is at least about 95%, 96%, 97%, 98%, 99% or 100% identical to the amino acid sequence of SEQ ID NO:13.

In one embodiment, the antigen binding receptor comprises in order from the N-terminus to the C-terminus
(i) a heavy chain variable domain (VH) that is at least about 95%, 96%, 97%, 98%, 99% or 100% identical to the amino acid sequence of SEQ ID NO:44,
(ii) a peptide linker, in particular the peptide linker of SEQ ID NO:16,
(iii) a light chain variable domain (VL) that is at least about 95%, 96%, 97%, 98%, 99% or 100% identical to the amino acid sequence of SEQ ID NO:9,
(iv) a peptide linker, in particular the peptide linker of SEQ ID NO:19,
(v) an anchoring transmembrane domain, in particular an anchoring transmembrane domain that is at least about 95%, 96%, 97%, 98%, 99% or 100% identical to the amino acid sequence of SEQ ID NO:11,
(vi) a co-stimulatory signaling domain, in particular a co-stimulatory signaling domain that is at least about 95%, 96%, 97%, 98%, 99% or 100% identical to the amino acid sequence of SEQ ID NO:12, and
(vii) a stimulatory signaling domain, in particular a stimulatory signaling domain that is at least about 95%, 96%, 97%, 98%, 99% or 100% identical to the amino acid sequence of SEQ ID NO:13.

In one embodiment, the antigen binding receptor comprises an amino acid sequence that is at least about 95%, 96%, 97%, 98%, 99% or 100% identical to the amino acid sequence of: SEQ ID NO:7. In one embodiment, the antigen binding receptor comprises the amino acid sequence of: SEQ ID NO: 7.

In one embodiment, the antigen binding receptor comprises an amino acid sequence that is at least about 95%, 96%, 97%, 98%, 99% or 100% identical to the amino acid sequence of: SEQ ID NO:125. In one embodiment, the antigen binding receptor comprises the amino acid sequence of: SEQ ID NO:125.

In one embodiment, the antigen binding receptor comprises an amino acid sequence that is at least about 95%, 96%, 97%, 98%, 99% or 100% identical to the amino acid sequence of: SEQ ID NO:127. In one embodiment, provided is an antigen binding receptor comprising the amino acid sequence of: SEQ ID NO:127.

In one embodiment, the antigen binding receptor is fused to a reporter protein, particularly to GFP or enhanced analogs thereof. In one embodiment, the antigen binding receptor is fused at the C-terminus to the N-terminus of eGFP (enhanced green fluorescent protein), optionally through a peptide linker as described herein. In a preferred embodiment, the peptide linker is GEGRGSLLTCGDVEENPGP (T2A) of SEQ ID NO:18.

### Transduced Cells Capable of Expressing Antigen Binding Receptors

A further aspect of the present invention are transduced T cells capable of expressing an antigen binding receptor as herein described. These antigen binding receptors relate to molecules which are naturally not comprised in and/or on the surface of T cells and which are not (endogenously) expressed in or on normal (non-transduced) T cells. Thus, the antigen binding receptor in and/or on T cells is artificially introduced into T cells. In the context of the present invention said T cells, preferably CD8+ T cells, may be isolated/obtained from a subject to be treated as defined herein. Accordingly, the antigen binding receptors as described herein which are artificially introduced and subsequently presented in and/or on the surface of said T cells comprise domains comprising one or more antigen binding moiety accessible (in vitro or in vivo) to (Ig-derived) immunoglobulins, preferably antibodies, in particular to the Fc domain of the antibodies. In the context of the present invention, these artificially introduced molecules are presented in and/or on the surface of said T cells after (retroviral, lentiviral or non-viral) transduction as described herein below. Accordingly, after transduction, T cells according to the invention can be activated by immunoglobulins, preferably antibodies comprising specific mutations in the Fc domain as described herein and in the presence of target cells.

The invention also relates to transduced T cells expressing an antigen binding receptor encoded by (a) nucleic acid molecule(s) encoding the antigen binding receptor of the present invention. Accordingly, in the context of the present invention, the transduced cell may comprise a nucleic acid molecule encoding the antigen binding receptor of the present invention or a vector of the present invention which expresses an antigen binding receptor of the present invention.

In the context of the present invention, the term “transduced T cell” relates to a genetically modified T cell (i.e. a T cell wherein a nucleic acid molecule has been introduced deliberately). The herein provided transduced T cell may comprise the vector of the present invention. Preferably, the herein provided transduced T cell comprises the nucleic acid molecule encoding the antigen binding receptor of the present invention and/or the vector of the present invention. The transduced T cell of the invention may be a T cell which transiently or stably expresses the foreign DNA (i.e. the nucleic acid molecule which has been introduced into the T cell). In particular, the nucleic acid molecule encoding the antigen binding receptor of the present invention can be stably integrated into the genome of the T cell by using a retroviral or lentiviral transduction. By using mRNA transfection, the nucleic acid molecule encoding the antigen binding receptor of the present invention may be expressed transiently. Preferably, the herein provided transduced T cell has been genetically modified by introducing a nucleic acid molecule in the T cell via a viral vector (e.g. a retroviral vector or a lentiviral vector). Accordingly, the expression of the antigen binding receptors may be constitutive and the extracellular domain of the antigen binding receptor may be detectable on the cell surface. This extracellular domain of the antigen binding receptor may comprise the complete extracellular domain of an antigen binding receptor as defined herein but also parts thereof. The minimal size required being the antigen binding site of the antigen binding moiety in the antigen binding receptor.

The expression may also be conditional or inducible in the case that the antigen binding receptor is introduced into T cells under the control of an inducible or repressible promoter. Examples for such inducible or repressible promoters can be a transcriptional system containing the alcohol dehydrogenase I (alcA) gene promoter and the transactivator protein AlcR. Different agricultural alcohol-based formulations are used to control the expression of a gene of interest linked to the alcA promoter. Furthermore, tetracycline-responsive promoter systems can function either to activate or repress gene expression system in the presence of tetracycline. Some of the elements of the systems include a tetracycline repressor protein (TetR), a tetracycline operator sequence (tetO) and a tetracycline transactivator fusion protein (tTA), which is the fusion of TetR and a herpes simplex virus protein 16 (VP16) activation sequence. Further, steroid-responsive promoters, metal-regulated or pathogenesis-related (PR) protein related promoters can be used.

The expression can be constitutive or constitutional, depending on the system used. The antigen binding receptors of the present invention can be expressed on the surface of the herein provided transduced T cell. The extracellular portion of the antigen binding receptor (i.e. the extracellular domain of the antigen binding receptor can be detected on the cell surface, while the intracellular portion (i.e. the co-stimulatory signaling domain(s) and the stimulatory signaling domain) are not detectable on the cell surface. The detection of the extracellular domain of the antigen binding receptor can be carried out by using an antibody which specifically binds to this extracellular domain or by the mutated Fc domain which the extracellular domain is capable to bind. The extracellular domain can be detected using these antibodies or Fc domains by flow cytometry or microscopy.

Other cells can also be transduced with the antigen binding receptors of the invention and thereby be directed against target cells. These further cells include but are not limited to B-cells, Natural Killer (NK) cells, innate lymphoid cells, macrophages, monocytes, dendritic cells, or neutrophils. Preferentially, said immune cell would be a lymphocyte. Triggering of the antigen binding receptor of the present invention on the surface of the leukocyte will render the cell cytotoxic against a target cell in conjunction with an antibody comprising a heterodimeric Fc domain irrespective of the lineage the cell originated from. Cytotoxicity will happen irrespective of the stimulatory signaling domain or co-stimulatory signaling domain chosen for the antigen binding receptor and is not dependent on the exogenous supply of additional cytokines. Accordingly, the transduced cell of the present invention may be, e.g., a CD4+ T cell, a CD8+-T cell, a γδ T cell, a Natural Killer (NK) T cell, a Natural Killer (NK) cell, a tumor-infiltrating lymphocyte (TIL) cell, a myeloid cell, or a mesenchymal stem cell. Preferably, the herein provided transduced cell is a T cell (e.g. an autologous T cell), more preferably, the transduced cell is a CD8+ T cell. Accordingly, in the context of the present invention, the transduced cell is a CD8+ T cell. Further, in the context of the present invention, the transduced cell is an autologous T cell. Accordingly, in the context of the present invention, the transduced cell is preferably an autologous CD8+ T cell. In addition to the use of autologous cells (e.g. T cells) isolated from the subject, the present invention also comprehends the use of allogeneic cells. Accordingly, in the context of the present invention the transduced cell may also be an allogeneic cell, such as an allogeneic CD8+ T cell. The term allogeneic refers to cells coming from an unrelated donor individual/subject which is human leukocyte antigen (HLA) compatible to the individual/subject which will be treated by e.g. the herein described antigen binding receptor expressing transduced cell. Autologous cells refer to cells which are isolated/obtained as described herein above from the subject to be treated with the transduced cell described herein.

The transduced cell of the invention may be co-transduced with further nucleic acid molecules, e.g. with a nucleic acid molecule encoding a cytokine.

The present invention also relates to a method for the production of a transduced T cell expressing an antigen binding receptor of the invention, comprising the steps of transducing a T cell with a vector of the present invention, culturing the transduced T cell under conditions allowing the expressing of the antigen binding receptor in or on said transduced cell and recovering said transduced T cell.

In the context of the present invention, the transduced cell of the present invention is preferably produced by isolating cells (e.g., T cells, preferably CD8+ T cells) from a subject (preferably a human patient). Methods for isolating/obtaining cells (e.g. T cells, preferably CD8+ T cells) from patients or from donors are well known in the art and in the context of the present cells (e.g. T cells, preferably CD8+ T cells) from patients or from donors, e.g. cells may be isolated by blood draw or removal of bone marrow. After isolating/obtaining cells as a sample of the patient, the cells (e.g. T cells) are separated from the other ingredients of the sample. Several methods for separating cells (e.g. T cells) from the sample are known and include, without being limiting, e.g. leukapheresis for obtaining cells from the peripheral blood sample from a patient or from a donor, isolating/obtaining cells by using a FACS cell sorting apparatus. The isolated/obtained cells T cells, are subsequently cultivated and expanded, e.g., by using an anti-CD3 antibody, by using anti-CD3 and anti-CD28 monoclonal antibodies and/or by using an anti-CD3 antibody, an anti-CD28 antibody and interleukin-2 (IL-2) (see, e.g., Dudley, Immunother. 26 (2003), 332-342 or Dudley, Clin. Oncol. 26 (2008), 5233-5239).

In a subsequent step the cells (e.g. T cells) are artificially/genetically modified/transduced by methods known in the art (see, e.g., Lemoine, J Gene Med 6 (2004), 374-386). Methods for transducing cells (e.g. T cells) are known in the art and include, without being limited, in a case where nucleic acid or a recombinant nucleic acid is transduced, for example, an electroporation method, calcium phosphate method, cationic lipid method or liposome method. The nucleic acid to be transduced can be conventionally and highly efficiently transduced by using a commercially available transfection reagent, for example, Lipofectamine (manufactured by Invitrogen, catalogue no.: 11668027). In a case where a vector is used, the vector can be transduced in the same manner as the above-mentioned nucleic acid as long as the vector is a plasmid vector (i.e. a vector which is not a viral vector In the context of the present invention, the methods for transducing cells (e.g. T cells) include retroviral or lentiviral T cell transduction, non-viral vectors (e.g., sleeping beauty minicircle vector) as well as mRNA transfection. “mRNA transfection” refers to a method well known to those skilled in the art to transiently express a protein of interest, like in the present case the antigen binding receptor of the present invention, in a cell to be transduced. In brief cells may be electroporated with the mRNA coding for the antigen binding receptor of the present by using an electroporation system (such as e.g. Gene Pulser, Bio-Rad) and thereafter cultured by standard cell (e.g. T cell) culture protocol as described above (see Zhao et al., Mol Ther. 13(1) (2006), 151-159.) The transduced cell of the invention can be generated by lentiviral, or most preferably retroviral transduction.

In this context, suitable retroviral vectors for transducing cells are known in the art such as SAMEN CMV/SRa (Clay et al., J. Immunol. 163 (1999), 507-513), LZRS-id3-IHRES (Heemskerk et al., J. Exp. Med. 186 (1997), 1597-1602), FeLV (Neil et al., Nature 308 (1984), 814-820), SAX (Kantoff et al., Proc. Natl. Acad. Sci. USA 83 (1986), 6563-6567), pDOL (Desiderio, J. Exp. Med. 167 (1988), 372-388), N2 (Kasid et al., Proc. Natl. Acad. Sci. USA 87 (1990), 473-477), LNL6 (Tiberghien et al., Blood 84 (1994), 1333-1341), pZipNEO (Chen et al., J. Immunol. 153 (1994), 3630-3638), LASN (Mullen et al., Hum. Gene Ther. 7 (1996), 1123-1129), pG1XsNa (Taylor et al., J. Exp. Med. 184 (1996), 2031-2036), LCNX (Sun et al., Hum. Gene Ther. 8 (1997), 1041-1048), SFG (Gallardo et al., Blood 90 (1997), and LXSN (Sun et al., Hum. Gene Ther. 8 (1997), 1041-1048), SFG (Gallardo et al., Blood 90 (1997), 952-957), HMB-Hb-Hu (Vieillard et al., Proc. Natl. Acad. Sci. USA 94 (1997), 11595-11600), pMV7 (Cochlovius et al., Cancer Immunol. Immunother. 46 (1998), 61-66), pSTITCH (Weitjens et al., Gene Ther 5 (1998), 1195-1203), pLZR (Yang et al., Hum. Gene Ther. 10 (1999), 123-132), pBAG (Wu et al., Hum. Gene Ther. 10 (1999), 977-982), rKat.43.267bn (Gilham et al., J. Immunother. 25 (2002), 139-151), pLGSN (Engels et al., Hum. Gene Ther. 14 (2003), 1155-1168), pMP71 (Engels et al., Hum. Gene Ther. 14 (2003), 1155-1168), pGCSAM (Morgan et al., J. Immunol. 171 (2003), 3287-3295), pMSGV (Zhao et al., J. Immunol. 174 (2005), 4415-4423), or pMX (de Witte et al., J. Immunol. 181 (2008), 5128-5136). In the context of the present invention, suitable lentiviral vector for transducing cells (e.g. T cells) are, e.g. PL-SIN lentiviral vector (Hotta et al., Nat Methods. 6(5) (2009), 370-376), p156RRL-sinPPT-CMV-GFP-PRE/NheI (Campeau et al., PLoS One 4(8) (2009), e6529), pCMVR8.74 (Addgene Catalogoue No.: 22036), FUGW (Lois et al., Science 295(5556) (2002), 868-872, pLVX-EF1 (Addgene Catalogue No.: 64368), pLVE (Brunger et al., Proc Natl Acad Sci USA 111(9) (2014), E798-806), pCDH1-MCS1-EF1 (Hu et al., Mol Cancer Res. 7(11) (2009), 1756-1770), pSLIK (Wang et al., Nat Cell Biol. 16(4) (2014), 345-356), pLJM1 (Solomon et al., Nat Genet. 45(12) (2013), 1428-30), pLX302 (Kang et al., Sci Signal. 6(287) (2013), rs13), pHR-IG (Xie et al., J Cereb Blood Flow Metab. 33(12) (2013), 1875-85), pRRLSIN (Addgene Catalogoue No.: 62053), pLS (Miyoshi et al., J Virol. 72(10) (1998), 8150-8157), pLL3.7 (Lazebnik et al., J Biol Chem. 283(7) (2008), 11078-82), FRIG (Raissi et al., Mol Cell Neurosci. 57 (2013), 23-32), pWPT (Ritz-Laser et al., Diabetologia. 46(6) (2003), 810-821), pBOB (Marr et al., J Mol Neurosci. 22(1-2) (2004), 5-11), or pLEX (Addgene Catalogue No.: 27976).

The transduced cells of the present invention is/are preferably grown under controlled conditions, outside of their natural environment. In particular, the term “culturing” means that cells (e.g. the transduced cell(s) of the invention) which are derived from multicellular eukaryotes (preferably from a human patient) are grown in vitro. Culturing cells is a laboratory technique of keeping cells alive which are separated from their original tissue source. Herein, the transduced cell of the present invention is cultured under conditions allowing the expression of the antigen binding receptor of the present invention in or on said transduced cells. Conditions which allow the expression or a transgene (i.e. of the antigen binding receptor of the present invention) are commonly known in the art and include, e.g., agonistic anti-CD3- and anti-CD28 antibodies and the addition of cytokines such as interleukin 2 (IL-2), interleukin 7 (IL-7), interleukin 12 (IL-12) and/or interleukin 15 (IL-15). After expression of the antigen binding receptor of the present invention in the cultured transduced cell (e.g., a CD8+T), the transduced cell is recovered (i.e. re-extracted) from the culture (i.e. from the culture medium).

Accordingly, also encompassed by the invention is a transduced cell, preferably a T cell, in particular a CD8+T expressing an antigen binding receptor encoded by a nucleic acid molecule of the invention obtainable by the method of the present invention.

### Nucleic Acid Molecules

A further aspect of the present invention are nucleic acids and vectors encoding one or several antigen binding receptors as herein described. An exemplary nucleic acid molecules encoding the antigen binding receptors is shown in SEQ ID NOs:20. The nucleic acid molecules may be under the control of regulatory sequences. For example, promoters, transcriptional enhancers and/or sequences which allow for induced expression of the antigen binding receptor of the invention may be employed. In the context of the present invention, the nucleic acid molecules are expressed under the control of constitutive or inducible promoter. Suitable promoters are e.g. the CMV promoter (Qin et al., PLoS One 5(5) (2010), e10611), the UBC promoter (Qin et al., PLoS One 5(5) (2010), e10611), PGK (Qin et al., PLoS One 5(5) (2010), e10611), the EF1A promoter (Qin et al., PLoS One 5(5) (2010), e10611), the CAGG promoter (Qin et al., PLoS One 5(5) (2010), e10611), the SV40 promoter (Qin et al., PLoS One 5(5) (2010), e10611), the COPIA promoter (Qin et al., PLoS One 5(5) (2010), e10611), the ACT5C promoter (Qin et al., PLoS One 5(5) (2010), e10611), the TRE promoter (Qin et al., PLoS One. 5(5) (2010), e10611), the Oct3/4 promoter (Chang et al., Molecular Therapy 9 (2004), S367-S367 (doi: 10.1016/j.ymthe.2004.06.904)), or the Nanog promoter (Wu et al., Cell Res. 15(5) (2005), 317-24). The present invention therefore also relates to (a) vector(s) comprising the nucleic acid molecule(s) described in the present invention. Herein the term vector relates to a circular or linear nucleic acid molecule which can autonomously replicate in a cell into which it has been introduced. Many suitable vectors are known to those skilled in molecular biology, the choice of which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook et al. (loc cit.) and Ausubel, Current Protocols in Molecular Biology, Green Publishing Associates and Wiley Interscience, N.Y. (1989), (1994). Alternatively, the polynucleotides and vectors of the invention can be reconstituted into liposomes for delivery to target cells. As discussed in further details below, a cloning vector was used to isolate individual sequences of DNA. Relevant sequences can be transferred into expression vectors where expression of a particular polypeptide is required. Typical cloning vectors include pBluescript SK, pGEM, pUC9, pBR322, pGA18 and pGBT9. Typical expression vectors include pTRE, pCAL-n-EK, pESP-1, pOP13CAT.

The invention also relates to (a) vector(s) comprising (a) nucleic acid molecule(s) which is (are) a regulatory sequence operably linked to said nucleic acid molecule(s) encoding an antigen binding receptor as defined herein. In the context of the present invention the vector can be polycistronic. Such regulatory sequences (control elements) are known to the skilled person and may include a promoter, a splice cassette, translation initiation codon, translation and insertion site for introducing an insert into the vector(s). In the context of the present invention, said nucleic acid molecule(s) is (are) operatively linked to said expression control sequences allowing expression in eukaryotic or prokaryotic cells. It is envisaged that said vector(s) is (are) (an) expression vector(s) comprising the nucleic acid molecule(s) encoding the antigen binding receptor as defined herein. Operably linked refers to a juxtaposition wherein the components so described are in a relationship permitting them to function in their intended manner. A control sequence operably linked to a coding sequence is ligated in such a way that expression of the coding sequence is achieved under conditions compatible with the control sequences. In case the control sequence is a promoter, it is obvious for a skilled person that double-stranded nucleic acid is preferably used.

In the context of the present invention the recited vector(s) is (are) an expression vector(s). An expression vector is a construct that can be used to transform a selected cell and provides for expression of a coding sequence in the selected cell. An expression vector(s) can for instance be cloning (a) vector(s), (a) binary vector(s) or (a) integrating vector(s). Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the PL, lac, trp or tac promoter in E. coli, and examples of regulatory elements permitting expression in eukaryotic host cells are the AOX1 or GAL1 promoter in yeast or the CMV-, SV40, RSV-promoter (Rous sarcoma virus), CMV-enhancer, SV40-enhancer or a globin intron in mammalian and other animal cells.

Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences encoding signal peptides capable of directing the polypeptide to a cellular compartment or secreting it into the medium may be added to the coding sequence of the recited nucleic acid sequence and are well known in the art; see also, e.g., appended Examples.

The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode an antigen binding receptor including an N-terminal identification peptide imparting desired characteristics, e.g., stabilization or simplified purification of expressed recombinant product; see supra. In this context, suitable expression vectors are known in the art such as Okayama-Berg cDNA expression vector pcDV1 (Pharmacia), pCDM8, pRc/CMV, pcDNA1, pcDNA3 (In-vitrogene), pEF-DHFR, pEF-ADA or pEF-neo (Raum et al. Cancer Immunol Immunother 50 (2001), 141-150) or pSPORT1 (GIBCO BRL).

In the context of the present invention, the expression control sequences will be eukaryotic promoter systems in vectors capable of transforming or transfecting eukaryotic cells, but control sequences for prokaryotic cells may also be used. Once the vector has been incorporated into the appropriate cell, the cell is maintained under conditions suitable for high level expression of the nucleotide sequences, and as desired. Additional regulatory elements may include transcriptional as well as translational enhancers. Advantageously, the above-described vectors of the invention comprise a selectable and/or scorable marker. Selectable marker genes useful for the selection of transformed cells and, e.g., plant tissue and plants are well known to those skilled in the art and comprise, for example, antimetabolite resistance as the basis of selection for dhfr, which confers resistance to methotrexate (Reiss, Plant Physiol. (Life Sci. Adv.) 13 (1994), 143-149), npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus which confers resistance to Blasticidin S (Tamura, Biosci. Biotechnol. Biochem. 59 (1995), 2336-2338).

Useful scorable markers are also known to those skilled in the art and are commercially available. Advantageously, said marker is a gene encoding luciferase (Giacomin, Pl. Sci. 116 (1996), 59-72; Scikantha, J. Bact. 178 (1996), 121), green fluorescent protein (Gerdes, FEBS Lett. 389 (1996), 44-47) or β-glucuronidase (Jefferson, EMBO J. 6 (1987), 3901-3907). This embodiment is particularly useful for simple and rapid screening of cells, tissues and organisms containing a recited vector.

As described above, the recited nucleic acid molecule(s) can be used alone or as part of (a) vector(s) to express the antigen binding receptors of the invention in cells, for, e.g., adoptive T cell therapy but also for gene therapy purposes. The nucleic acid molecules or vector(s) containing the DNA sequence(s) encoding any one of the herein described antigen binding receptors is introduced into the cells which in turn produce the polypeptide of interest. Gene therapy, which is based on introducing therapeutic genes into cells by ex-vivo or in-vivo techniques is one of the most important applications of gene transfer. Suitable vectors, methods or gene-delivery systems for in methods or gene-delivery systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Verma, Nature 389 (1994), 239; Isner, Lancet 348 (1996), 370-374; Muhlhauser, Circ. Res. 77 (1995), 1077-1086; Onodera, Blood 91 (1998), 30-36; Verma, Gene Ther. 5 (1998), 692-699; Nabel, Ann. N.Y. Acad. Sci. 811 (1997), 289-292; Verzeletti, Hum. Gene Ther. 9 (1998), 2243-51; Wang, Nature Medicine 2 (1996), 714-716; WO 94/29469; WO 97/00957; U.S. Pat. Nos. 5,580,859; 5,589,466; or Schaper, Current Opinion in Biotechnology 7 (1996), 635-640. The recited nucleic acid molecule(s) and vector(s) may be designed for direct introduction or for introduction via liposomes, or viral vectors (e.g., adenoviral, retroviral) into the cell. In the context of the present invention, said cell is a T cells, such as CD8+ T cells, CD4+ T cells, CD3+ T cells, 76 T cells or natural killer (NK) T cells, preferably CD8+ T cells.

In accordance with the above, the present invention relates to methods to derive vectors, particularly plasmids, cosmids and bacteriophages used conventionally in genetic engineering that comprise a nucleic acid molecule encoding the polypeptide sequence of an antigen binding receptor defined herein. In the context of the present invention, said vector is an expression vector and/or a gene transfer or targeting vector. Expression vectors derived from viruses such as retroviruses, vaccinia virus, adeno-associated virus, herpes virus, or bovine papilloma virus, may be used for delivery of the recited polynucleotides or vector into targeted cell populations.

Methods which are well known to those skilled in the art can be used to construct (a) recombinant vector(s); see, for example, the techniques described in Sambrook et al. (loc cit.), Ausubel (1989, loc cit.) or other standard text books. Alternatively, the recited nucleic acid molecules and vectors can be reconstituted into liposomes for delivery to target cells. The vectors containing the nucleic acid molecules of the invention can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra. The recited vector may, inter alia, be the pEF-DHFR, pEF-ADA or pEF-neo. The vectors pEF-DHFR, pEF-ADA and pEF-neo have been described in the art, e.g. in Mack et al. Proc. Natl. Acad. Sci. USA 92 (1995), 7021-7025 and Raum et al. Cancer Immunol Immunother 50 (2001), 141-150.

The invention also provides for a T cell transduced with a vector as described herein. Said T cell may be produced by introducing at least one of the above described vector or at least one of the above described nucleic acid molecules into the T cell or its precursor cell. The presence of said at least one vector or at least one nucleic acid molecule in the T cell mediates the expression of a gene encoding the above described antigen binding receptor comprising an extracellular domain comprising an antigen binding moiety capable of specific binding to a mutated Fc domain. The vector of the present invention can be polycistronic.

The described nucleic acid molecule(s) or vector(s) which is (are) introduced in the T cell or its precursor cell may either integrate into the genome of the cell or it may be maintained extrachromosomally.

### Kits

A further aspect of the present invention are kits comprising or consisting of (an) antibody/antibodies comprising a heterodimeric Fc domain according to the invention and (a) nucleic acid(s) encoding an antigen binding receptor according to the invention and/or cells, preferably T cells for transduction/transduced with said antigen binding receptors.

Accordingly, provided is a kit comprising
(a) an antibody comprising a heterodimeric Fc domain composed of a first and a second subunit, wherein the first subunit comprises the amino acid mutation P329G according to EU numbering, wherein the second subunit comprises a proline (P) at position 329 according to EU numbering.
(b) a transduced T cell capable of expressing an antigen binding receptor capable of specific binding to the first subunit.

Further provided is a kit comprising
(a) an antibody comprising a heterodimeric Fc domain composed of a first and a second subunit, wherein the first subunit comprises the amino acid mutation P329G according to EU numbering, wherein the second subunit comprises a proline (P) at position 329 according to EU numbering.
(b) an isolated polynucleotide encoding an antigen binding receptor capable of specific binding to the first subunit.

In a preferred embodiment, the kits of the present invention comprise transduced T cells, isolated polynucleotides and/or vectors and one or more antibodies comprising a heterodimeric Fc domain composed of a first and a second subunit, wherein the first subunit comprises the amino acid mutation P329G according to EU numbering and wherein the second subunit comprises a proline (P) at position 329 according to EU numbering. In particular, embodiments, the antibody is a therapeutic antibody, e.g. a tumor specific antibody as hereinbefore described. Tumor specific antigens are known in the art and hereinbefore described. In the context of the present invention, the antibody is administered before, simultaneously with or after administration of transduced T cell expressing an antigen binding receptor of the invention. The kits according to the present invention comprise transduced T cells or polynucleotides/vectors to generate transduced T cells. In this context, the transduced T cells are universal T cells since they are not specific to a given tumor but can be targeted to any tumor by use of an antibody comprising the heterodimeric Fc domain. Herein provided are examples of antibodies comprising a heterodimeric Fc domain comprising the amino acid mutation P329G according to EU numbering (for example SEQ ID Nos: 129-131), however, any antibody comprising a heterodimeric Fc domain composed of a first and a second subunit, wherein the first subunit comprises the amino acid mutation P329G according to EU numbering and wherein the second subunit comprises a proline (P) at position 329 according to EU numbering.

Parts of the kit of the invention can be packaged individually in vials or bottles or in combination in containers or multicontainer units. Additionally, the kit of the present invention may comprise a (closed) bag cell incubation system where patient cells, preferably T cells as described herein, can be transduced with (an) antigen binding receptor(s) of the invention and incubated under GMP (good manufacturing practice, as described in the guidelines for good manufacturing practice published by the European Commission under http://ec.europa.eu/health/documents/eudralex/index_en.htm) conditions. Furthermore, the kit of the present invention comprises a (closed) bag cell incubation system where isolated/obtained patients T cells can be transduced with (an) antigen binding receptor(s) of the invention and incubated under GMP. Furthermore, in the context of the present invention, the kit may also comprise a vector encoding (the) antigen binding receptor(s) as described herein. The kit of the present invention may be advantageously used, inter alia, for carrying out the method of the invention and could be employed in a variety of applications referred herein, e.g., as research tools or medical tools. The manufacture of the kits preferably follows standard procedures which are known to the person skilled in the art.

In this context, patient derived cells, preferably T cells, can be transduced with an antigen binding receptor of the invention capable of specific binding to a heterodimeric Fc domain comprising the amino acid mutation P329G according to EU numbering as described above. The extracellular domain comprising an antigen binding moiety capable of specific binding to a mutated heterodimeric Fc domain does not naturally occur in or on T cells. Accordingly, the patient derived cells transduced with the kits of the invention will acquire the capability of specific binding to the antibody comprising a heterodimeric Fc domain according to the invention, e.g. a therapeutic antibody, and will become capable of inducing elimination/lysis of target cells via interaction with said antibody, wherein said antibody is able to bind to a tumor-specific antigen naturally occurring (that is endogenously expressed) on the surface of a tumor cell. Binding of the extracellular domain of the antigen binding receptor as described herein activates that T cell and brings it into physical contact with the tumor cell through the antibody comprising the heterodimeric Fc domain. Non-transduced or endogenous T cells (e.g. CD8+ T cells) are unable to bind to the heterodimeric Fc domain of the antibody comprising the mutated Fc domain. The transduced T cells expressing the antigen binding receptor as herein described remain unaffected by a therapeutic antibody not comprising the mutations in the Fc domain as described herein. Accordingly, T cells expressing the antigen binding receptor molecule as herein described have the ability to lyse target cells in the presence of an antibody as herein described comprising a heterodimeric Fc domain in vivo and/or in vitro. Corresponding target cells comprise cells expressing a surface molecule, i.e. a tumor-specific antigen naturally occurring on the surface of a tumor cell, which is recognized by at least one, preferably two, binding domains of the antibody as described herein.

Lysis of the target cell can be detected by methods known in the art. Accordingly, such methods comprise, inter alia, physiological in vitro assays. Such physiological assays may monitor cell death, for example by loss of cell membrane integrity (e.g. FACS based propidium Iodide assay, trypan blue influx assay, photometric enzyme release assays (LDH), radiometric 51Cr release assay, fluorometric Europium release and CalceinAM release assays). Further assays comprise monitoring of cell viability, for example by photometric MTT, XTT, WST-1 and alamarBlue assays, radiometric 3H-Thd incorporation assay, clonogenic assay measuring cell division activity, and fluorometric Rhodamine123 assay measuring mitochondrial transmembrane gradient. In addition, apoptosis may be monitored for example by FACS-based phosphatidylserin exposure assay, ELISA-based TUNEL test, caspase activity assay (photometric, fluorometric or ELISA-based) or analyzing changed cell morphology (shrinking, membrane blebbing).

### Combination Therapy

The molecules or constructs (e.g., antibodies, antigen binding receptors, transduced T cells and kits) provided herein are particularly useful in medical settings, in particular for treatment of cancer. For example a tumor may be treated with a transduced T cell expressing an antigen binding receptor according to the invention in conjunction with a therapeutic antibody that binds to a target antigen on the tumor cell and comprising a heterodimeric Fc domain. Accordingly, in certain embodiments, the antibodies, the antigen binding receptor, the transduced T cell or the kit are used in the treatment of cancer, in particular cancer of epithelial, endothelial or mesothelial origin and cancer of the blood.

The tumor specificity of the treatment is provided by the antibody comprising the heterodimeric Fc domain and specificity to binds to a target cell antigen. The antibody can be administered before, simultaneously with or after administration of transduced T cell expressing an antigen binding receptor of the invention. In this context, the transduced T cells are universal T cells since they are not specific for a given tumor but can target any tumor depending on the specificity of the antibody used according to the invention.

The cancer may be a cancer/carcinoma of epithelial, endothelial or mesothelial origin or a cancer of the blood. In one embodiment the cancer/carcinoma is selected from the group consisting of gastrointestinal cancer, pancreatic cancer, cholangiocellular cancer, lung cancer, breast cancer, ovarian cancer, skin cancer, oral cancer, gastric cancer, cervical cancer, B and T cell lymphoma, myeloid leukemia, ovarial cancer, leukemia, lymphatic leukemia, nasopharyngeal carcinoma, colon cancer, prostate cancer, renal cell cancer, head and neck cancer, skin cancer (melanoma), cancers of the genitourinary tract, e.g., testis cancer, ovarial cancer, endothelial cancer, cervix cancer and kidney cancer, cancer of the bile duct, esophagus cancer, cancer of the salivatory glands and cancer of the thyroid gland or other tumorous diseases like haematological tumors, gliomas, sarcomas or osteosarcomas.

For example, tumorous diseases and/or lymphomas may be treated with a specific construct directed against these medical indication(s). For example, gastrointestinal cancer, pancreatic cancer, cholangiocellular cancer, lung cancer, breast cancer, ovarian cancer, skin cancer and/or oral cancer may be treated with an antibody directed against (human) EpCAM (as the tumor-specific antigen naturally occurring on the surface of a tumor cell).

Gastrointestinal cancer, pancreatic cancer, cholangiocellular cancer, lung cancer, breast cancer, ovarian cancer, skin cancer and/or oral cancer may be treated with a transduced T cell of the present invention administered before, simultaneously with or after administration of an antibody comprising a heterodimeric Fc domain and directed against HER1, preferably human HER1. Furthermore, gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of an antibody comprising a heterodimeric Fc domain and directed against MCSP, preferably human MCSP. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of an antibody comprising a heterodimeric Fc domain and directed against FOLR1, preferably human FOLR1. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of an antibody comprising a heterodimeric Fc domain and directed against Trop-2, preferably human Trop-2. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of an antibody comprising a heterodimeric Fc domain and directed against PSCA, preferably human PSCA. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of an antibody comprising a heterodimeric Fc domain and directed against EGFRvIII, preferably human EGFRvIII. Gastrointestinal cancer, pancreatic cancer, cholangiocellular cancer, lung cancer, breast cancer, ovarian cancer, skin cancer, glioblastoma and/or oral cancer may be treated with a transduced T cell of the present invention administered before, simultaneously with or after administration of an antibody comprising a heterodimeric Fc domain and directed against MSLN, preferably human MSLN. Gastric cancer, breast cancer and/or cervical cancer may be treated with a transduced T cell of the present invention administered before, simultaneously with or after administration of an antibody comprising a heterodimeric Fc domain and directed against HER2, preferably human HER2. Gastric cancer and/or lung cancer may be treated with a transduced T cell of the present invention administered before, simultaneously with or after administration of an antibody comprising a heterodimeric Fc domain and directed against HER3, preferably human HER3. B-cell lymphoma and/or T cell lymphoma may be treated with a transduced T cell of the present invention administered before, simultaneously with or after administration of an antibody comprising a heterodimeric Fc domain and directed against CD20, preferably human CD20. B-cell lymphoma and/or T cell lymphoma may be treated with a transduced T cell of the present invention administered before, simultaneously with or after administration of an antibody comprising a heterodimeric Fc domain and directed against CD22, preferably human CD22. Myeloid leukemia may be treated with a transduced T cell of the present invention administered before, simultaneously with or after administration of an antibody comprising a heterodimeric Fc domain and directed against CD33, preferably human CD33. Ovarian cancer, lung cancer, breast cancer and/or gastrointestinal cancer may be treated with a transduced T cell of the present invention administered before, simultaneously with or after administration of an antibody comprising a heterodimeric Fc domain and directed against CA12-5, preferably human CA12-5. Gastrointestinal cancer, leukemia and/or nasopharyngeal carcinoma may be treated with a transduced T cell of the present invention administered before, simultaneously with or after administration of an antibody comprising a heterodimeric Fc domain and directed against HLA-DR, preferably human HLA-DR. Colon cancer, breast cancer, ovarian cancer, lung cancer and/or pancreatic cancer may be with a transduced T cell of the present invention administered before, simultaneously with or after administration of an antibody comprising a heterodimeric Fc domain and directed against MUC-1, preferably human MUC-1. Colon cancer may be treated with a transduced T cell of the present invention administered before, simultaneously with or after administration of an antibody comprising a heterodimeric Fc domain and directed against A33, preferably human A33. Prostate cancer may be treated with a transduced T cell of the present invention administered before, simultaneously with or after administration of an antibody comprising a heterodimeric Fc domain and directed against PSMA, preferably human PSMA. Gastrointestinal cancer, pancreatic cancer, cholangiocellular cancer, lung cancer, breast cancer, ovarian cancer, skin cancer and/or oral cancer may be treated with a transduced T cell of the present invention administered before, simultaneously with or after administration of an antibody comprising a heterodimeric Fc domain directed against the transferrin receptor, preferably the human transferring receptor. Pancreatic cancer, lung cancer and/or breast cancer may be treated with a transduced T cell of the present invention administered before, simultaneously with or after administration of an antibody comprising a heterodimeric Fc domain and directed against the transferrin receptor, preferably the human transferring receptor. Renal cancer may be with a transduced T cell of the present invention administered before, simultaneously with or after administration of an antibody comprising a heterodimeric Fc domain and directed against CA-IX, preferably human CA-IX.

The invention also relates to a method for the treatment of a disease, a malignant disease such as cancer of epithelial, endothelial or mesothelial origin and/or cancer of blood. In the context of the present invention, said subject is a human.

In the context of the present invention a particular method for the treatment of a disease comprises the steps of
(a) isolating T cells, preferably CD8+ T cells, from a subject;
(b) transducing said isolated T cells, preferably CD8+ T cells, with an antigen binding receptor as described herein; and
(c) administering the transduced T cells, preferably CD8+ T cells, to said subject.

In the context of the present invention, said transduced T cells, preferably CD8+ T cells, and/or heterodimeric antibody/antibodies are co-administered to said subject by intravenous infusion.

Moreover, in the context of the present invention the present invention, provides a method for the treatment of a disease comprising the steps of
(a) isolating T cells, preferably CD8+ T cells, from a subject;
(b) transducing said isolated T cells, preferably CD8+ T cells, with an antigen binding receptor as described herein;
(c) optionally co-transducing said isolated T cells, preferably CD8+ T cells, with a T cell receptor;
(d) expanding the T cells, preferably CD8+ T cells, by anti-CD3 and anti-CD28 antibodies; and
(e) administering the transduced T cells, preferably CD8+ T cells, to said subject.

The above mentioned step (d) (referring to the expanding step of the T cells such as TIL by anti-CD3 and/or anti-CD28 antibodies) may also be performed in the presence of (stimulating) cytokines such as interleukin-2 and/or interleukin-15 (IL-15). In the context of the present invention, the above mentioned step (d) (referring to the expanding step of the T cells such as TIL by anti-CD3 and/or anti-CD28 antibodies) may also be performed in the presence of interleukin-12 (IL-12), interleukin-7 (IL-7) and/or interleukin-21 (IL-21).

The method for the treatment, in addition, comprise the administration of the antibody used according to the present invention. Said antibody may be administered before, simultaneously with or after the transduced T cells are to be administered. In the context of the present invention the administration of the transduced T cells will be performed by intravenous infusion. In the context of the present invention that transduced T cells are isolated/obtained from the subject to be treated.

The invention further envisages the co-administration protocols with other compounds, e.g., molecules capable of providing an activation signal for immune effector cells, for cell proliferation or for cell stimulation. Said molecule may be, e.g., a further primary activation signal for T cells (e.g. a further costimulatory molecule: molecules of B7 family, Ox40L, 4.1 BBL, CD40L, anti-CTLA-4, anti-PD-1), or a further cytokine interleukin (e.g., IL-2).

The composition of the invention as described above may also be a diagnostic composition further comprising, optionally, means and methods for detection.

These and other embodiments are disclosed and encompassed by the description and Examples of the present invention. Further literature concerning any one of the antibodies, cells, methods, uses and compounds to be employed in accordance with the present invention may be retrieved from public libraries and databases, using for example electronic devices. For example, the public database “Medline”, available on the Internet, may be utilized, for example under http://www.ncbi.nlm.nih.gov/PubMed/medline.html. Further databases and addresses, such as http://www.ncbi.nlm.nih.gov/, http://www.tigr.org/, http://www.infobiogen.fr/, and http://www.fmi.ch/biology/research_tools.html, are known to the person skilled in the art and can also be obtained using, e.g., http://www.lycos.com.

### Exemplary Sequences

TABLE 2
Exemplary VH3VL1 P329G-CAR amino acid
sequences: CDR definition according to Kabat

| | | SEQ |
|---|---|---|
| | | ID |
| Construct | Amino acid sequence | NO |
| VH3 CDR H1 | RYWMN | 1 |
| VH3 CDR H2 | EITPDSSTINYAPSLKG | 2 |
| VH3 CDR H3 | PYDYGAWFAS | 3 |
| VL1 CDR L1 | RSSTGAVTTSNYAN | 4 |
| VL1 CDR L2 | GTNKRAP | 5 |
| VL1 CDR L3 | ALWYSNHWV | 6 |
| VH3VL1- | EVQLVESGGGLVQPGGSLRLSCAASGFTFSRYW | 7 |
| CD8ATD- | MNWVRQAPGKGLEWVGEITPDSSTINYAPSLKG | |
| CD137CSD- | RFTISRDNAKNSLYLQMNSLRAEDTAVYYCARP | |
| CD3zSSD | YDYGAWFASWGQGTLVTVSSGGGGSGGGGSGGG | |
| fusion | GSGGGGSQAVVTQEPSLTVSPGGTVTLTCRSST | |
| | GAVTTSNYANWVQEKPDHLFTGLIGGTNKRAPG | |
| | TPARFSGSLLGGKAALTLSGAQPEDEAEYYCAL | |
| | WYSNHWVFGGGTKLTVLGGGGSLKPTTTPAPRP | |
| | PTPAPTIASQPLSLRPEACRPAAGGAVHTRGLD | |
| | FACDIYIWAPLAGTCGVLLLSLVITKRGRKKLL | |
| | YIFKQPFMRPVQTTQEEDGCSCRFPEEEEGGCE | |
| | LRVKFSRSADAPAYQQGQNQLYNELNLGRREEY | |
| | DVLDKRRGRDPEMGGKPRRKNPQEGLYNELQKD | |
| | KMAEAYSEIGMKGERRRGKGHDGLYQGLSTATK | |
| | DTYDALHMQALPPR | |
| VH3 VH | EVQLVESGGGLVQPGGSLRLSCAASGFTFSRYW | 8 |
| | MNWVRQAPGKGLEWVGEITPDSSTINYAPSLKG | |
| | RFTISRDNAKNSLYLQMNSLRAEDTAVYYCARP | |
| | YDYGAWFASWGQGTLVTVSS | |
| VL1 VL | QAVVTQEPSLTVSPGGTVTLTCRSSTGAVTTSN | 9 |
| | YANWVQEKPDHLFTGLIGGTNKRAPGTPARFSG | |
| | SLLGGKAALTLSGAQPEDEAEYYCALWYSNHWV | |
| | FGGGTKLTVL | |
| VH3VL1 | EVQLVESGGGLVQPGGSLRLSCAASGFTFSRYW | 10 |
| scFv | MNWVRQAPGKGLEWVGEITPDSSTINYAPSLKG | |
| | RFTISRDNAKNSLYLQMNSLRAEDTAVYYCARP | |
| | YDYGAWFASWGQGTLVTVSSGGGGSGGGGSGGG | |
| | GSGGGGSQAVVTQEPSLTVSPGGTVTLTCRSST | |
| | GAVTTSNYANWVQEKPDHLFTGLIGGTNKRAPG | |
| | TPARFSGSLLGGKAALTLSGAQPEDEAEYYCAL | |
| | WYSNHWVFGGGTKLTVL | |
| CD8ATD | IYIWAPLAGTCGVLLLSLVIT | 11 |
| CD137CSD | KRGRKKLLYIFKQPFMRPVQTTQEEDGCSCRFP | 12 |
| | EEEEGGCEL | |
| CD3zSSD | RVKFSRSADAPAYQQGQNQLYNELNLGRREEYD | 13 |
| | VLDKRRGRDPEMGGKPRRKNPQEGLYNELQKDK | |
| | MAEAYSEIGMKGERRRGKGHDGLYQGLSTATKD | |
| | TYDALHMQALPPR | |
| CD28ATD- | KPTTTPAPRPPTPAPTIASQPLSLRPEACRPAA | 14 |
| CD137CSD- | GGAVHTRGLDFACDIYIWAPLAGTCGVLLLSLV | |
| CD3zSSD | ITKRGRKKLLYIFKQPFMRPVQTTQEEDGCSCR | |
| | FPEEEEGGCELRVKFSRSADAPAYQQGQNQLYN | |
| | ELNLGRREEYDVLDKRRGRDPEMGGKPRRKNPQ | |
| | EGLYNELQKDKMAEAYSEIGMKGERRRGKGHDG | |
| | LYQGLSTATKDTYDALHMQALPPR | |
| eGFP | VSKGEELFTGVVPILVELDGDVNGHKFSVSGEG | 15 |
| | EGDATYGKLTLKFICTTGKLPVPWPTLVTTLTY | |
| | GVQCFSRYPDHMKQHDFFKSAMPEGYVQERTIF | |
| | FKDDGNYKTRAEVKFEGDTLVNRIELKGIDFKE | |
| | DGNILGHKLEYNYNSHNVYIMADKQKNGIKVNF | |
| | KIRHNIEDGSVQLADHYQQNTPIGDGPVLLPDN | |
| | HYLSTQSALSKDPNEKRDHMVLLEFVTAAGITL | |
| | GMDELYK | |
| (G4S)4 | GGGGSGGGGSGGGGSGGGGS | 16 |
| linker | | |
| G4S linker | GGGGS | 17 |
| T2A linker | GEGRGSLLTCGDVEENPGP | 18 |
| CD8stalk | KPTTTPAPRPPTPAPTIASQPLSLRPEACRPAA | 19 |
| | GGAVHTRGLDFACD | |

TABLE 3
Exemplary VH3 x VL1 P329G-CAR DNA sequences:

| | | SEQ |
|---|---|---|
| Con- | | ID |
| struct | DNA sequence | NO |
| VH3VL1- | GAGGTGCAGCTGGTGGAGAGCGGCGGCGGCCTGGTGC | 20 |
| CD28ATD- | AGCCCGGCGGCAGCCTGAGGCTGAGCTGCGCCGCCAG | |
| CD137- | CGGCTTCACCTTCAGCAGGTACTGGATGAACTGGGTG | |
| CSD- | AGGCAGGCCCCCGGCAAGGGCCTGGAGTGGGTGGGCG | |
| CD3zSSD | AGATCACCCCCGACAGCAGCACCATCAACTACGCCCC | |
| fusion | CAGCCTGAAGGGCAGGTTCACCATCAGCAGGGACAAC | |
| | GCCAAGAACAGCCTGTACCTGCAGATGAACAGCCTGA | |
| | GGGCCGAGGACACCGCCGTGTACTACTGCGCCAGGCC | |
| | CTACGACTACGGCGCCTGGTTCGCCAGCTGGGGCCAG | |
| | GGCACCCTGGTGACCGTGAGCAGCGGAGGGGGCGGAA | |
| | GTGGTGGCGGGGGAAGCGGCGGGGGTGGCAGCGGAGG | |
| | GGGCGGATCTCAGGCCGTGGTGACCCAGGAGCCCAGC | |
| | CTGACCGTGAGCCCCGGCGGCACCGTGACCCTGACCT | |
| | GCAGGAGCAGCACCGGCGCCGTGACCACCAGCAACTA | |
| | CGCCAACTGGGTGCAGGAGAAGCCCGACCACCTGTTC | |
| | ACCGGCCTGATCGGCGGCACCAACAAGAGGGCCCCCG | |
| | GCACCCCCGCCAGGTTCAGCGGCAGCCTGCTGGGCGG | |
| | CAAGGCCGCCCTGACCCTGAGCGGCGCCCAGCCCGAG | |
| | GACGAGGCCGAGTACTACTGCGCCCTGTGGTACAGCA | |
| | ACCACTGGGTGTTCGGCGGCGGCACCAAGCTGACCGT | |
| | CCTAGGAGGGGGCGGATCCTTGAAGCCCACCACGACG | |
| | CCAGCGCCGCGACCACCAACACCGGCGCCCACCATCG | |
| | CGTCGCAGCCCCTGTCCCTGCGCCCAGAGGCGTGCCG | |
| | GCCAGCGGCGGGGGGCGCAGTGCACACGAGGGGGCTG | |
| | GACTTCGCCTGTGATATCTACATCTGGGCGCCCCTGG | |
| | CCGGGACTTGTGGGGTCCTTCTCCTGTCACTGGTTAT | |
| | CACCAAACGGGGCAGAAAGAAACTCCTGTATATATTC | |
| | AAACAACCATTTATGAGACCAGTACAAACTACTCAAG | |
| | AGGAAGATGGCTGTAGCTGCCGATTTCCAGAAGAAGA | |
| | AGAAGGAGGATGTGAACTGAGAGTGAAGTTCAGCAGG | |
| | AGCGCAGACGCCCCCGCGTACCAGCAGGGCCAGAACC | |
| | AGCTCTATAACGAGCTCAATCTAGGACGAAGAGAGGA | |
| | GTACGATGTTTTGGACAAGAGACGTGGCCGGGACCCT | |
| | GAGATGGGGGGAAAGCCGAGAAGGAAGAACCCTCAGG | |
| | AAGGCCTGTACAATGAACTGCAGAAAGATAAGATGGC | |
| | GGAGGCCTACAGTGAGATTGGGATGAAAGGCGAGCGC | |
| | CGGAGGGGCAAGGGGCACGATGGCCTTTACCAGGGTC | |
| | TCAGTACAGCCACCAAGGACACCTACGACGCCCTTCA | |
| | CATGCAGGCCCTGCCCCCTCGC | |
| VH3 | GAGGTGCAGCTGGTGGAGAGCGGCGGCGGCCTGGTGC | 21 |
| | AGCCCGGCGGCAGCCTGAGGCTGAGCTGCGCCGCCAG | |
| | CGGCTTCACCTTCAGCAGGTACTGGATGAACTGGGTG | |
| | AGGCAGGCCCCCGGCAAGGGCCTGGAGTGGGTGGGCG | |
| | AGATCACCCCCGACAGCAGCACCATCAACTACGCCCC | |
| | CAGCCTGAAGGGCAGGTTCACCATCAGCAGGGACAAC | |
| | GCCAAGAACAGCCTGTACCTGCAGATGAACAGCCTGA | |
| | GGGCCGAGGACACCGCCGTGTACTACTGCGCCAGGCC | |
| | CTACGACTACGGCGCCTGGTTCGCCAGCTGGGGCCAG | |
| | GGCACCCTGGTGACCGTGAGCAGC | |
| VL1 | CAGGCCGTGGTGACCCAGGAGCCCAGCCTGACCGTGA | 22 |
| | GCCCCGGCGGCACCGTGACCCTGACCTGCAGGAGCAG | |
| | CACCGGCGCCGTGACCACCAGCAACTACGCCAACTGG | |
| | GTGCAGGAGAAGCCCGACCACCTGTTCACCGGCCTGA | |
| | TCGGCGGCACCAACAAGAGGGCCCCCGGCACCCCCGC | |
| | CAGGTTCAGCGGCAGCCTGCTGGGCGGCAAGGCCGCC | |
| | CTGACCCTGAGCGGCGCCCAGCCCGAGGACGAGGCCG | |
| | AGTACTACTGCGCCCTGTGGTACAGCAACCACTGGGT | |
| | GTTCGGCGGCGGCACCAAGCTGACCGTCCTA | |
| VH3VL1 | GAGGTGCAGCTGGTGGAGAGCGGCGGCGGCCTGGTGC | 23 |
| scFv | AGCCCGGCGGCAGCCTGAGGCTGAGCTGCGCCGCCAG | |
| | CGGCTTCACCTTCAGCAGGTACTGGATGAACTGGGTG | |
| | AGGCAGGCCCCCGGCAAGGGCCTGGAGTGGGTGGGCG | |
| | AGATCACCCCCGACAGCAGCACCATCAACTACGCCCC | |
| | CAGCCTGAAGGGCAGGTTCACCATCAGCAGGGACAAC | |
| | GCCAAGAACAGCCTGTACCTGCAGATGAACAGCCTGA | |
| | GGGCCGAGGACACCGCCGTGTACTACTGCGCCAGGCC | |
| | CTACGACTACGGCGCCTGGTTCGCCAGCTGGGGCCAG | |
| | GGCACCCTGGTGACCGTGAGCAGCGGAGGGGGCGGAA | |
| | GTGGTGGCGGGGGAAGCGGCGGGGGTGGCAGCGGAGG | |
| | GGGCGGATCTCAGGCCGTGGTGACCCAGGAGCCCAGC | |
| | CTGACCGTGAGCCCCGGCGGCACCGTGACCCTGACCT | |
| | GCAGGAGCAGCACCGGCGCCGTGACCACCAGCAACTA | |
| | CGCCAACTGGGTGCAGGAGAAGCCCGACCACCTGTTC | |
| | ACCGGCCTGATCGGCGGCACCAACAAGAGGGCCCCCG | |
| | GCACCCCCGCCAGGTTCAGCGGCAGCCTGCTGGGCGG | |
| | CAAGGCCGCCCTGACCCTGAGCGGCGCCCAGCCCGAG | |
| | GACGAGGCCGAGTACTACTGCGCCCTGTGGTACAGCA | |
| | ACCACTGGGTGTTCGGCGGCGGCACCAAGCTGACCGT | |
| | CCTA | |
| CD8ATD | ATCTACATCTGGGCGCCCCTGGCCGGGACTTGTGGGG | 24 |
| | TCCTTCTCCTGTCACTGGTTATCACC | |
| CD137CSD | AAACGGGGCAGAAAGAAACTCCTGTATATATTCAAAC | 25 |
| | AACCATTTATGAGACCAGTACAAACTACTCAAGAGGA | |
| | AGATGGCTGTAGCTGCCGATTTCCAGAAGAAGAAGAA | |
| | GGAGGATGTGAACTG | |
| CD3zSSD | AGAGTGAAGTTCAGCAGGAGCGCAGACGCCCCCGCGT | 26 |
| | ACCAGCAGGGCCAGAACCAGCTCTATAACGAGCTCAA | |
| | TCTAGGACGAAGAGAGGAGTACGATGTTTTGGACAAG | |
| | AGACGTGGCCGGGACCCTGAGATGGGGGGAAAGCCGA | |
| | GAAGGAAGAACCCTCAGGAAGGCCTGTACAATGAACT | |
| | GCAGAAAGATAAGATGGCGGAGGCCTACAGTGAGATT | |
| | GGGATGAAAGGCGAGCGCCGGAGGGGCAAGGGGCACG | |
| | ATGGCCTTTACCAGGGTCTCAGTACAGCCACCAAGGA | |
| | CACCTACGACGCCCTTCACATGCAGGCCCTGCCCCCT | |
| | CGC | |
| CD28ATD- | ATCTACATCTGGGCGCCCCTGGCCGGGACTTGTGGGG | 27 |
| CD137- | TCCTTCTCCTGTCACTGGTTATCACCAAACGGGGCAG | |
| CSD- | AAAGAAACTCCTGTATATATTCAAACAACCATTTATG | |
| CD3zSSD | AGACCAGTACAAACTACTCAAGAGGAAGATGGCTGTA | |
| | GCTGCCGATTTCCAGAAGAAGAAGAAGGAGGATGTGA | |
| | ACTGAGAGTGAAGTTCAGCAGGAGCGCAGACGCCCCC | |
| | GCGTACCAGCAGGGCCAGAACCAGCTCTATAACGAGC | |
| | TCAATCTAGGACGAAGAGAGGAGTACGATGTTTTGGA | |
| | CAAGAGACGTGGCCGGGACCCTGAGATGGGGGGAAAG | |
| | CCGAGAAGGAAGAACCCTCAGGAAGGCCTGTACAATG | |
| | AACTGCAGAAAGATAAGATGGCGGAGGCCTACAGTGA | |
| | GATTGGGATGAAAGGCGAGCGCCGGAGGGGCAAGGGG | |
| | CACGATGGCCTTTACCAGGGTCTCAGTACAGCCACCA | |
| | AGGACACCTACGACGCCCTTCACATGCAGGCCCTGCC | |
| | CCCTCGC | |
| T2A | TCCGGAGAGGGCAGAGGAAGTCTTCTAACATGCGGTG | 28 |
| element | ACGTGGAGGAGAATCCCGGCCCTAGG | |
| eGFP | GTGAGCAAGGGCGAGGAGCTGTTCACCGGGGTGGTGC | 29 |
| | CCATCCTGGTCGAGCTGGACGGCGACGTAAACGGCCA | |
| | CAAGTTCAGCGTGTCCGGCGAGGGCGAGGGCGATGCC | |
| | ACCTACGGCAAGCTGACCCTGAAGTTCATCTGCACCA | |
| | CCGGCAAGCTGCCCGTGCCCTGGCCCACCCTCGTGAC | |
| | CACCCTGACCTACGGCGTGCAGTGCTTCAGCCGCTAC | |
| | CCCGACCACATGAAGCAGCACGACTTCTTCAAGTCCG | |
| | CCATGCCCGAAGGCTACGTCCAGGAGCGCACCATCTT | |
| | CTTCAAGGACGACGGCAACTACAAGACCCGCGCCGAG | |
| | GTGAAGTTCGAGGGCGACACCCTGGTGAACCGCATCG | |
| | AGCTGAAGGGCATCGACTTCAAGGAGGACGGCAACAT | |
| | CCTGGGGCACAAGCTGGAGTACAACTACAACAGCCAC | |
| | AACGTCTATATCATGGCCGACAAGCAGAAGAACGGCA | |
| | TCAAGGTGAACTTCAAGATCCGCCACAACATCGAGGA | |
| | CGGCAGCGTGCAGCTCGCCGACCACTACCAGCAGAAC | |
| | ACCCCCATCGGCGACGGCCCCGTGCTGCTGCCCGACA | |
| | ACCACTACCTGAGCACCCAGTCCGCCCTGAGCAAAGA | |
| | CCCCAACGAGAAGCGCGATCACATGGTCCTGCTGGAG | |
| | TTCGTGACCGCCGCCGGGATCACTCTCGGCATGGACG | |
| | AGCTGTACAAGTGA | |
| VH3VL1- | GAGGTGCAGCTGGTGGAGAGCGGCGGCGGCCTGGTGC | 30 |
| CD28ATD- | AGCCCGGCGGCAGCCTGAGGCTGAGCTGCGCCGCCAG | |
| CD137- | CGGCTTCACCTTCAGCAGGTACTGGATGAACTGGGTG | |
| CSD- | AGGCAGGCCCCCGGCAAGGGCCTGGAGTGGGGGGCGA | |
| CD3zSSD | GATCACCCCCGACAGCAGCACCATCAACTACGCCCCC | |
| GFP | AGCCTGAAGGGCAGGTTCACCATCAGCAGGGACAACG | |
| fusion | CCAAGAACAGCCTGTACCTGCAGATGAACAGCCTGAG | |
| | GGCCGAGGACACCGCCGTGTACTACTGCGCCAGGCCC | |
| | TACGACTACGGCGCCTGGTTCGCCAGCTGGGGCCAGG | |
| | GCACCCTGGTGACCGTGAGCAGCGGAGGGGGCGGAAG | |
| | TGGTGGCGGGGGAAGCGGCGGGGGTGGCAGCGGAGGG | |
| | GGCGGATCTCAGGCCGTGGTGACCCAGGAGCCCAGCC | |
| | TGACCGTGAGCCCCGGCGGCACCGTGACCCTGACCTG | |
| | CAGGAGCAGCACCGGCGCCGTGACCACCAGCAACTAC | |
| | GCCAACTGGGTGCAGGAGAAGCCCGACCACCTGTTCA | |
| | CCGGCCTGATCGGCGGCACCAACAAGAGGGCCCCCGG | |
| | CACCCCCGCCAGGTTCAGCGGCAGCCTGCTGGGCGGC | |
| | AAGGCCGCCCTGACCCTGAGCGGCGCCCAGCCCGAGG | |
| | ACGAGGCCGAGTACTACTGCGCCCTGTGGTACAGCAA | |
| | CCACTGGGTGTTCGGCGGCGGCACCAAGCTGACCGTC | |
| | CTAGGAGGGGGCGGATCCTTGAAGCCCACCACGACGC | |
| | CAGCGCCGCGACCACCAACACCGGCGCCCACCATCGC | |
| | GTCGCAGCCCCTGTCCCTGCGCCCAGAGGCGTGCCGG | |
| | CCAGCGGCGGGGGGCGCAGTGCACACGAGGGGGCTGG | |
| | ACTTCGCCTGTGATATCTACATCTGGGCGCCCCTGGC | |
| | CGGGACTTGTGGGGTCCTTCTCCTGTCACTGGTTATC | |
| | ACCAAACGGGGCAGAAAGAAACTCCTGTATATATTCA | |
| | AACAACCATTTATGAGACCAGTACAAACTACTCAAGA | |
| | GGAAGATGGCTGTAGCTGCCGATTTCCAGAAGAAGAA | |
| | GAAGGAGGATGTGAACTGAGAGTGAAGTTCAGCAGGA | |
| | GCGCAGACGCCCCCGCGTACCAGCAGGGCCAGAACCA | |
| | GCTCTATAACGAGCTCAATCTAGGACGAAGAGAGGAG | |
| | TACGATGTTTTGGACAAGAGACGTGGCCGGGACCCTG | |
| | AGATGGGGGGAAAGCCGAGAAGGAAGAACCCTCAGGA | |
| | AGGCCTGTACAATGAACTGCAGAAAGATAAGATGGCG | |
| | GAGGCCTACAGTGAGATTGGGATGAAAGGCGAGCGCC | |
| | GGAGGGGCAAGGGGCACGATGGCCTTTACCAGGGTCT | |
| | CAGTACAGCCACCAAGGACACCTACGACGCCCTTCAC | |
| | ATGCAGGCCCTGCCCCCTCGCGAATTCTCCGGAGAGG | |
| | GCAGAGGAAGTCTTCTAACATGCGGTGACGTGGAGGA | |
| | GAATCCCGGCCCTAGGGTGAGCAAGGGCGAGGAGCTG | |
| | TTCACCGGGGTGGTGCCCATCCTGGTCGAGCTGGACG | |
| | GCGACGTAAACGGCCACAAGTTCAGCGTGTCCGGCGA | |
| | GGGCGAGGGCGATGCCACCTACGGCAAGCTGACCCTG | |
| | AAGTTCATCTGCACCACCGGCAAGCTGCCCGTGCCCT | |
| | GGCCCACCCTCGTGACCACCCTGACCTACGGCGTGCA | |
| | GTGCTTCAGCCGCTACCCCGACCACATGAAGCAGCAC | |
| | GACTTCTTCAAGTCCGCCATGCCCGAAGGCTACGTCC | |
| | AGGAGCGCACCATCTTCTTCAAGGACGACGGCAACTA | |
| | CAAGACCCGCGCCGAGGTGAAGTTCGAGGGCGACACC | |
| | CTGGTGAACCGCATCGAGCTGAAGGGCATCGACTTCA | |
| | AGGAGGACGGCAACATCCTGGGGCACAAGCTGGAGTA | |
| | CAACTACAACAGCCACAACGTCTATATCATGGCCGAC | |
| | AAGCAGAAGAACGGCATCAAGGTGAACTTCAAGATCC | |
| | GCCACAACATCGAGGACGGCAGCGTGCAGCTCGCCGA | |
| | CCACTACCAGCAGAACACCCCCATCGGCGACGGCCCC | |
| | GTGCTGCTGCCCGACAACCACTACCTGAGCACCCAGT | |
| | CCGCCCTGAGCAAAGACCCCAACGAGAAGCGCGATCA | |
| | CATGGTCCTGCTGGAGTTCGTGACCGCCGCCGGGATC | |
| | ACTCTCGGCATGGACGAGCTGTACAAG | |

TABLE 4
Exemplary VLIVH3 P329G-CAR amino acid
sequences: CDR definition to Kabat

| | | SEQ |
|---|---|---|
| | | ID |
| Construct | Amino acid sequence | NO |
| VH3 CDR H1 | see Table 2 | 1 |
| VH3 CDR H2 | see Table 2 | 2 |
| VH3 CDR H3 | see Table 2 | 3 |
| VL1 CDR L1 | see Table 2 | 4 |
| VL1 CDR L2 | see Table 2 | 5 |
| VL1 CDR L3 | see Table 2 | 6 |
| VL1VH3- | QAVVTQEPSLTVSPGGTVTLTCRSSTGAVTTSN | 31 |
| CD8ATD- | YANWVQEKPDHLFTGLIGGTNKRAPGTPARFSG | |
| CD137CSD- | SLLGGKAALTLSGAQPEDEAEYYCALWYSNHWV | |
| CD3zSSD | FGGGTKLTVLGGGGSGGGGSGGGGSGGGGSEVQ | |
| fusion | LVESGGGLVQPGGSLRLSCAASGFTFSRYWMNW | |
| | VRQAPGKGLEWVGEITPDSSTINYAPSLKGRFT | |
| | ISRDNAKNSLYLQMNSLRAEDTAVYYCARPYDY | |
| | GAWFASWGQGTLVTVSSGGGGSLKPTTTPAPRP | |
| | PT | |
| VH3 VH | See Table 2 | 8 |
| VL1 VL | See Table 2 | 9 |
| VL1VH3 | MLLLVTSLLLCELPHPAFLLIPAQAVVTQEPSL | 32 |
| scFv | TVSPGGTVTLTCRSSTGAVTTSNYANWVQEKPD | |
| | HLFTGLIGGTNKRAPGTPARFSGSLLGGKAALT | |
| | LSGAQPEDEAEYYCALWYSNHWVFGGGTKLTVL | |
| | GGGGSGGGGSGGGGSGGGGSEVQLVESGGGLVQ | |
| | PGGSLRLSCAASGFTFSRYWMNWVRQAPGKGLE | |
| | WVGEITPDSSTINYAPSLKGRFTISRDNAKNSL | |
| | YLQMNSLRAEDTAVYYCARPYDYGAWFASWGQG | |
| | TLVTVSS | |
| CD8ATD | see Table 2 | 11 |
| CD137CSD | see Table 2 | 12 |
| CD3zSSD | see Table 2 | 13 |
| CD28ATD- | see Table 2 | 14 |
| CD137CDS- | | |
| CD3zSSD | | |
| eGFP | see Table 2 | 15 |
| (G4S)4 | see Table 2 | 16 |
| linker | | |
| G4S linker | see Table 2 | 17 |
| T2A linker | see Table 2 | 18 |

TABLE 5
Exemplary VLIVH3 P329G-CAR DNA sequences:

| | | SEQ |
|---|---|---|
| Con- | | ID |
| struct | DNA sequence | NO |
| VL1VH3 | CAGGCCGTGGTGACCCAGGAGCCCAGCCTGACCGTGA | 33 |
| CD8ATD- | GCCCCGGCGGCACCGTGACCCTGACCTGCAGGAGCAG | |
| CD137- | CACCGGCGCCGTGACCACCAGCAACTACGCCAACTGG | |
| CSD- | GTGCAGGAGAAGCCCGACCACCTGTTCACCGGCCTGA | |
| CD3zSSD | TCGGCGGCACCAACAAGAGGGCCCCCGGCACCCCCGC | |
| fusion | CAGGTTCAGCGGCAGCCTGCTGGGCGGCAAGGCCGCC | |
| | CTGACCCTGAGCGGCGCCCAGCCCGAGGACGAGGCCG | |
| | AGTACTACTGCGCCCTGTGGTACAGCAACCACTGGGT | |
| | GTTCGGCGGCGGCACCAAGCTGACCGTCCTAGGAGGG | |
| | GGCGGAAGTGGTGGCGGGGGAAGCGGCGGGGGTGGCA | |
| | GCGGAGGGGGCGGATCTGAGGTGCAGCTGGTGGAGAG | |
| | CGGCGGCGGCCTGGTGCAGCCCGGCGGCAGCCTGAGG | |
| | CTGAGCTGCGCCGCCAGCGGCTTCACCTTCAGCAGGT | |
| | ACTGGATGAACTGGGTGAGGCAGGCCCCCGGCAAGGG | |
| | CCTGGAGTGGGTGGGCGAGATCACCCCCGACAGCAGC | |
| | ACCATCAACTACGCCCCCAGCCTGAAGGGCAGGTTCA | |
| | CCATCAGCAGGGACAACGCCAAGAACAGCCTGTACCT | |
| | GCAGATGAACAGCCTGAGGGCCGAGGACACCGCCGTG | |
| | TACTACTGCGCCAGGCCCTACGACTACGGCGCCTGGT | |
| | TCGCCAGCTGGGGCCAGGGCACCCTGGTGACCGTGAG | |
| | CAGCGGAGGGGGCGGATCCTTGAAGCCCACCACGACG | |
| | CCAGCGCCGCGACCACCAACACCGGCGCCCACCATCG | |
| | CGTCGCAGCCCCTGTCCCTGCGCCCAGAGGCGTGCCG | |
| | GCCAGCGGCGGGGGGCGCAGTGCACACGAGGGGGCTG | |
| | GACTTCGCCTGTGATATCTACATCTGGGCGCCCCTGG | |
| | CCGGGACTTGTGGGGTCCTTCTCCTGTCACTGGTTAT | |
| | CACCAAACGGGGCAGAAAGAAACTCCTGTATATATTC | |
| | AAACAACCATTTATGAGACCAGTACAAACTACTCAAG | |
| | AGGAAGATGGCTGTAGCTGCCGATTTCCAGAAGAAGA | |
| | AGAAGGAGGATGTGAACTGAGAGTGAAGTTCAGCAGG | |
| | AGCGCAGACGCCCCCGCGTACCAGCAGGGCCAGAACC | |
| | AGCTCTATAACGAGCTCAATCTAGGACGAAGAGAGGA | |
| | GTACGATGTTTTGGACAAGAGACGTGGCCGGGACCCT | |
| | GAGATGGGGGGAAAGCCGAGAAGGAAGAACCCTCAGG | |
| | AAGGCCTGTACAATGAACTGCAGAAAGATAAGATGGC | |
| | GGAGGCCTACAGTGAGATTGGGATGAAAGGCGAGCGC | |
| | CGGAGGGGCAAGGGGCACGATGGCCTTTACCAGGGTC | |
| | TCAGTACAGCCACCAAGGACACCTACGACGCCCTTCA | |
| | CATGCAGGCCCTGCCCCCTCGC | |
| VH3 | See Table 3 | 20 |
| VL1 | See Table 3 | 21 |
| CD8ATD | see Table 3 | 24 |
| CD137CSD | see Table 3 | 25 |
| CD3zSSD | see Table 3 | 26 |
| CD8ATD- | see Table 3 | 27 |
| CD137- | | |
| CSD- | | |
| CD3zSSD | | |
| T2A | see Table 3 | 28 |
| element | | |
| eGFP | see Table 3 | 29 |
| VL1VH3- | CAGGCCGTGGTGACCCAGGAGCCCAGCCTGACCGTGA | 34 |
| CD8ATD- | GCCCCGGCGGCACCGTGACCCTGACCTGCAGGAGCAG | |
| CD137- | CACCGGCGCCGTGACCACCAGCAACTACGCCAACTGG | |
| CSD- | GTGCAGGAGAAGCCCGACCACCTGTTCACCGGCCTGA | |
| CD3zSSD- | TCGGCGGCACCAACAAGAGGGCCCCCGGCACCCCCGC | |
| eGFP | CAGGTTCAGCGGCAGCCTGCTGGGCGGCAAGGCCGCC | |
| fusion | CTGACCCTGAGCGGCGCCCAGCCCGAGGACGAGGCCG | |
| | AGTACTACTGCGCCCTGTGGTACAGCAACCACTGGGT | |
| | GTTCGGCGGCGGCACCAAGCTGACCGTCCTAGGAGGG | |
| | GGCGGAAGTGGTGGCGGGGGAAGCGGCGGGGGTGGCA | |
| | GCGGAGGGGGCGGATCTGAGGTGCAGCTGGTGGAGAG | |
| | CGGCGGCGGCCTGGTGCAGCCCGGCGGCAGCCTGAGG | |
| | CTGAGCTGCGCCGCCAGCGGCTTCACCTTCAGCAGGT | |
| | ACTGGATGAACTGGGTGAGGCAGGCCCCCGGCAAGGG | |
| | CCTGGAGTGGGTGGGCGAGATCACCCCCGACAGCAGC | |
| | ACCATCAACTACGCCCCCAGCCTGAAGGGCAGGTTCA | |
| | CCATCAGCAGGGACAACGCCAAGAACAGCCTGTACCT | |
| | GCAGATGAACAGCCTGAGGGCCGAGGACACCGCCGTG | |
| | TACTACTGCGCCAGGCCCTACGACTACGGCGCCTGGT | |
| | TCGCCAGCTGGGGCCAGGGCACCCTGGTGACCGTGAG | |
| | CAGCGGAGGGGGCGGATCCTTGAAGCCCACCACGACG | |
| | CCAGCGCCGCGACCACCAACACCGGCGCCCACCATCG | |
| | CGTCGCAGCCCCTGTCCCTGCGCCCAGAGGCGTGCCG | |
| | GCCAGCGGCGGGGGGCGCAGTGCACACGAGGGGGCTG | |
| | GACTTCGCCTGTGATATCTACATCTGGGCGCCCCTGG | |
| | CCGGGACTTGTGGGGTCCTTCTCCTGTCACTGGTTAT | |
| | CACCAAACGGGGCAGAAAGAAACTCCTGTATATATTC | |
| | AAACAACCATTTATGAGACCAGTACAAACTACTCAAG | |
| | AGGAAGATGGCTGTAGCTGCCGATTTCCAGAAGAAGA | |
| | AGAAGGAGGATGTGAACTGAGAGTGAAGTTCAGCAGG | |
| | AGCGCAGACGCCCCCGCGTACCAGCAGGGCCAGAACC | |
| | AGCTCTATAACGAGCTCAATCTAGGACGAAGAGAGGA | |
| | GTACGATGTTTTGGACAAGAGACGTGGCCGGGACCCT | |
| | GAGATGGGGGGAAAGCCGAGAAGGAAGAACCCTCAGG | |
| | AAGGCCTGTACAATGAACTGCAGAAAGATAAGATGGC | |
| | GGAGGCCTACAGTGAGATTGGGATGAAAGGCGAGCGC | |
| | CGGAGGGGCAAGGGGCACGATGGCCTTTACCAGGGTC | |
| | TCAGTACAGCCACCAAGGACACCTACGACGCCCTTCA | |
| | CATGCAGGCCCTGCCCCCTCGCGAATTCTCCGGAGAG | |
| | GGCAGAGGAAGTCTTCTAACATGCGGTGACGTGGAGG | |
| | AGAATCCCGGCCCTAGGGTGAGCAAGGGCGAGGAGCT | |
| | GTTCACCGGGGTGGTGCCCATCCTGGTCGAGCTGGAC | |
| | GGCGACGTAAACGGCCACAAGTTCAGCGTGTCCGGCG | |
| | AGGGCGAGGGCGATGCCACCTACGGCAAGCTGACCCT | |
| | GAAGTTCATCTGCACCACCGGCAAGCTGCCCGTGCCC | |
| | TGGCCCACCCTCGTGACCACCCTGACCTACGGCGTGC | |
| | AGTGCTTCAGCCGCTACCCCGACCACATGAAGCAGCA | |
| | CGACTTCTTCAAGTCCGCCATGCCCGAAGGCTACGTC | |
| | CAGGAGCGCACCATCTTCTTCAAGGACGACGGCAACT | |
| | ACAAGACCCGCGCCGAGGTGAAGTTCGAGGGCGACAC | |
| | CCTGGTGAACCGCATCGAGCTGAAGGGCATCGACTTC | |
| | AAGGAGGACGGCAACATCCTGGGGCACAAGCTGGAGT | |
| | ACAACTACAACAGCCACAACGTCTATATCATGGCCGA | |
| | CAAGCAGAAGAACGGCATCAAGGTGAACTTCAAGATC | |
| | CGCCACAACATCGAGGACGGCAGCGTGCAGCTCGCCG | |
| | ACCACTACCAGCAGAACACCCCCATCGGCGACGGCCC | |
| | CGTGCTGCTGCCCGACAACCACTACCTGAGCACCCAG | |
| | TCCGCCCTGAGCAAAGACCCCAACGAGAAGCGCGATC | |
| | ACATGGTCCTGCTGGAGTTCGTGACCGCCGCCGGGAT | |
| | CACTCTCGGCATGGACGAGCTGTACAAG | |

TABLE 6
exemplary anti-P329G antibodies
CDR definition according to Kabat
Anti-P329G (M-1.7.24) huIgG1

| | | |
|---|---|---|
| HCDR1 | RYWMN | 1 |
| HCDR2 | EITPDSSTINYTPSLKD | 35 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVKLLESGGGLVQPGGSLKLSCAASGFDFSRYWMNWVRQ | 36 |
| | APGKGLEWIGEITPDSSTINYTPSLKDKFIISRDNAKNT | |
| | LYLQMIKVRSEDTALYYCVRPYDYGAWFASWGQGTLVTV | |
| | SA | |
| VL | QAVVTQESALTTSPGETVTLTCRSSTGAVTTSNYANWVQ | 37 |
| | EKPDHLFTGLIGGTNKRAPGVPARFSGSLIGDKAALTIT | |
| | GAQTEDEAIYFCALWYSNHWVFGGGTKLTVL | |
| HC | EVKLLESGGGLVQPGGSLKLSCAASGFDFSRYWMNWVRQ | 38 |
| | APGKGLEWIGEITPDSSTINYTPSLKDKFIISRDNAKNT | |
| | LYLQMIKVRSEDTALYYCVRPYDYGAWFASWGQGTLVTV | |
| | SAASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCDKTHTCPPCPAP | |
| | ELLGGPSVFLFPPKPKDTLMISRTPEVTCVVVDVSHEDP | |
| | EVKFNWYVDGVEVHNAKTKPREEQYNSTYRVVSVLTVLH | |
| | QDWLNGKEYKCKVSNKALPAPIEKTISKAKGQPREPQVY | |
| | TLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSP | |
| LC | QAVVTQESALTTSPGETVTLTCRSSTGAVTTSNYANWVQ | 39 |
| | EKPDHLFTGLIGGTNKRAPGVPARFSGSLIGDKAALTIT | |
| | GAQTEDEAIYFCALWYSNHWVFGGGTKLTVLGQPKAAPS | |
| | VTLFPPSSEELQANKATLVCLISDFYPGAVTVAWKADSS | |
| | PVKAGVETTTPSKQSNNKYAASSYLSLTPEQWKSHRSYS | |
| | CQVTHEGSTVEKTVAPTECS | |

Anti-P329G (VH1VL1) huIgG1

| | | |
|---|---|---|
| HCDR1 | RYWMN | 1 |
| HCDR2 | EITPDSSTINYTPSLKG | 40 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVQLVESGGGLVQPGGSLRLSCAASGFDFSRYWMNWVRQ | 41 |
| | APGKGLEWVGEITPDSSTINYTPSLKGRFTISRDNAKNS | |
| | LYLQMNSLRAEDTAVYYCVRPYDYGAWFASWGQGTLVTV | |
| | SS | |
| VL | QAVVTQEPSLTVSPGGTVTLTCRSSTGAVTTSNYANWVQ | 9 |
| | EKPDHLFTGLIGGTNKRAPGTPARFSGSLLGGKAALTLS | |
| | GAQPEDEAEYYCALWYSNHWVFGGGTKLTVL | |
| HC | EVQLVESGGGLVQPGGSLRLSCAASGFDFSRYWMNWVRQ | 42 |
| | APGKGLEWVGEITPDSSTINYTPSLKGRFTISRDNAKNS | |
| | LYLQMNSLRAEDTAVYYCVRPYDYGAWFASWGQGTLVTV | |
| | SSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCDKTHTCPPCPAP | |
| | ELLGGPSVFLFPPKPKDTLMISRTPEVTCVVVDVSHEDP | |
| | EVKFNWYVDGVEVHNAKTKPREEQYNSTYRVVSVLTVLH | |
| | QDWLNGKEYKCKVSNKALPAPIEKTISKAKGQPREPQVY | |
| | TLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSP | |
| LC | QAVVTQEPSLTVSPGGTVTLTCRSSTGAVTTSNYANWVQ | 43 |
| | EKPDHLFTGLIGGTNKRAPGTPARFSGSLLGGKAALTLS | |
| | GAQPEDEAEYYCALWYSNHWVFGGGTKLTVLGQPKAAPS | |
| | VTLFPPSSEELQANKATLVCLISDFYPGAVTVAWKADSS | |
| | PVKAGVETTTPSKQSNNKYAASSYLSLTPEQWKSHRSYS | |
| | CQVTHEGSTVEKTVAPTECS | |

Anti-P329G (VH2VL1) huIgG1

| | | |
|---|---|---|
| HCDR1 | RYWMN | 1 |
| HCDR2 | EITPDSSTINYAPSLKG | 2 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVQLVESGGGLVQPGGSLRLSCAASGFDFSRYWMNWVRQ | 44 |
| | APGKGLEWVGEITPDSSTINYAPSLKGRFTISRDNAKNS | |
| | LYLQMNSLRAEDTAVYYCVRPYDYGAWFASWGQGTLVTV | |
| | SS | |
| VL | QAVVTQEPSLTVSPGGTVTLTCRSSTGAVTTSNYANWVQ | 9 |
| | EKPDHLFTGLIGGTNKRAPGTPARFSGSLLGGKAALTLS | |
| | GAQPEDEAEYYCALWYSNHWVFGGGTKLTVL | |
| HC | EVQLVESGGGLVQPGGSLRLSCAASGFDFSRYWMNWVRQ | 45 |
| | APGKGLEWVGEITPDSSTINYAPSLKGRFTISRDNAKNS | |
| | LYLQMNSLRAEDTAVYYCVRPYDYGAWFASWGQGTLVTV | |
| | SSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCDKTHTCPPCPAP | |
| | ELLGGPSVFLFPPKPKDTLMISRTPEVTCVVVDVSHEDP | |
| | EVKFNWYVDGVEVHNAKTKPREEQYNSTYRVVSVLTVLH | |
| | QDWLNGKEYKCKVSNKALPAPIEKTISKAKGQPREPQVY | |
| | TLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSP | |
| LC | QAVVTQEPSLTVSPGGTVTLTCRSSTGAVTTSNYANWVQ | 43 |
| | EKPDHLFTGLIGGTNKRAPGTPARFSGSLLGGKAALTLS | |
| | GAQPEDEAEYYCALWYSNHWVFGGGTKLTVLGQPKAAPS | |
| | VTLFPPSSEELQANKATLVCLISDFYPGAVTVAWKADSS | |
| | PVKAGVETTTPSKQSNNKYAASSYLSLTPEQWKSHRSYS | |
| | CQVTHEGSTVEKTVAPTECS | |

Anti-P329G (VH3VL1) huIgG1

| | | |
|---|---|---|
| HCDR1 | RYWMN | 1 |
| HCDR2 | EITPDSSTINYAPSLKG | 2 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVQLVESGGGLVQPGGSLRLSCAASGFTFSRYWMNWVRQ | 8 |
| | APGKGLEWVGEITPDSSTINYAPSLKGRFTISRDNAKNS | |
| | LYLQMNSLRAEDTAVYYCARPYDYGAWFASWGQGTLVTV | |
| | SS | |
| VL | QAVVTQEPSLTVSPGGTVTLTCRSSTGAVTTSNYANWVQ | 9 |
| | EKPDHLFTGLIGGTNKRAPGTPARFSGSLLGGKAALTLS | |
| | GAQPEDEAEYYCALWYSNHWVFGGGTKLTVL | |
| HC | EVQLVESGGGLVQPGGSLRLSCAASGFTFSRYWMNWVRQ | 46 |
| | APGKGLEWVGEITPDSSTINYAPSLKGRFTISRDNAKNS | |
| | LYLQMNSLRAEDTAVYYCARPYDYGAWFASWGQGTLVTV | |
| | SSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCDKTHTCPPCPAP | |
| | ELLGGPSVFLFPPKPKDTLMISRTPEVTCVVVDVSHEDP | |
| | EVKFNWYVDGVEVHNAKTKPREEQYNSTYRVVSVLTVLH | |
| | QDWLNGKEYKCKVSNKALPAPIEKTISKAKGQPREPQVY | |
| | TLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSP | |
| LC | QAVVTQEPSLTVSPGGTVTLTCRSSTGAVTTSNYANWVQ | 43 |
| | EKPDHLFTGLIGGTNKRAPGTPARFSGSLLGGKAALTLS | |
| | GAQPEDEAEYYCALWYSNHWVFGGGTKLTVLGQPKAAPS | |
| | VTLFPPSSEELQANKATLVCLISDFYPGAVTVAWKADSS | |
| | PVKAGVETTTPSKQSNNKYAASSYLSLTPEQWKSHRSYS | |
| | CQVTHEGSTVEKTVAPTECS | |

Anti-P329G (VH4VL1) huIgG1

| | | |
|---|---|---|
| HCDR1 | RYWMN | 1 |
| HCDR2 | EITPDSSTINYADSVKG | 47 |
| HCDR3 | PYDYGAWFAS | 3 |
| LCDR1 | RSSTGAVTTSNYAN | 4 |
| LCDR2 | GTNKRAP | 5 |
| LCDR3 | ALWYSNHWV | 6 |
| VH | EVQLVESGGGLVQPGGSLRLSCAASGFDFSRYWMNWVRQ | 48 |
| | APGKGLEWVSEITPDSSTINYADSVKGRFTISRDNAKNS | |
| | LYLQMNSLRAEDTAVYYCARPYDYGAWFASWGQGTLVTV | |
| | SS | |
| VL | QAVVTQEPSLTVSPGGTVTLTCRSSTGAVTTSNYANWVQ | 9 |
| | EKPDHLFTGLIGGTNKRAPGTPARFSGSLLGGKAALTLS | |
| | GAQPEDEAEYYCALWYSNHWVFGGGTKLTVL | |
| HC | EVQLVESGGGLVQPGGSLRLSCAASGFDFSRYWMNWVRQ | 49 |
| | APGKGLEWVSEITPDSSTINYADSVKGRFTISRDNAKNS | |
| | LYLQMNSLRAEDTAVYYCARPYDYGAWFASWGQGTLVTV | |
| | SSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCDKTHTCPPCPAP | |
| | ELLGGPSVFLFPPKPKDTLMISRTPEVTCVVVDVSHEDP | |
| | EVKFNWYVDGVEVHNAKTKPREEQYNSTYRVVSVLTVLH | |
| | QDWLNGKEYKCKVSNKALPAPIEKTISKAKGQPREPQVY | |
| | TLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSP | |
| LC | QAVVTQEPSLTVSPGGTVTLTCRSSTGAVTTSNYANWVQ | 43 |
| | EKPDHLFTGLIGGTNKRAPGTPARFSGSLLGGKAALTLS | |
| | GAQPEDEAEYYCALWYSNHWVFGGGTKLTVLGQPKAAPS | |
| | VTLFPPSSEELQANKATLVCLISDFYPGAVTVAWKADSS | |
| | PVKAGVETTTPSKQSNNKYAASSYLSLTPEQWKSHRSYS | |
| | CQVTHEGSTVEKTVAPTECS | |

Anti-P329G (VH1VL2) huIgG1

| | | |
|---|---|---|
| HCDR1 | RYWMN | 1 |
| HCDR2 | EITPDSSTINYTPSLKG | 40 |
| HCDR3 | PYDYGAWFAS | 3 |
| LCDR1 | RSSTGAVTTSNYAN | 4 |
| LCDR2 | GTNKRAP | 5 |
| VH | EVQLVESGGGLVQPGGSLRLSCAASGFDFSRYWMNWVRQ | 41 |
| | APGKGLEWVGEITPDSSTINYTPSLKGRFTISRDNAKNS | |
| | LYLQMNSLRAEDTAVYYCVRPYDYGAWFASWGQGTLVTV | |
| | SS | |
| VL | QAVVTQEPSLTVSPGGTVTLTCRSSTGAVTTSNYANWFQ | 50 |
| | QKPGQAFTGLIGGTNKRAPGTPARFSGSLLGGKAALTLS | |
| | GAQPEDEAEYYCALWYSNHWVFGGGTKLTVL | |
| HC | EVQLVESGGGLVQPGGSLRLSCAASGFDFSRYWMNWVRQ | 42 |
| | APGKGLEWVGEITPDSSTINYTPSLKGRFTISRDNAKNS | |
| | LYLQMNSLRAEDTAVYYCVRPYDYGAWFASWGQGTLVTV | |
| | SSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCDKTHTCPPCPAP | |
| | ELLGGPSVFLFPPKPKDTLMISRTPEVTCVVVDVSHEDP | |
| | EVKFNWYVDGVEVHNAKTKPREEQYNSTYRVVSVLTVLH | |
| | QDWLNGKEYKCKVSNKALPAPIEKTISKAKGQPREPQVY | |
| | TLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSP | |
| LC | QAVVTQEPSLTVSPGGTVTLTCRSSTGAVTTSNYANWFQ | 51 |
| | QKPGQAFTGLIGGTNKRAPGTPARFSGSLLGGKAALTLS | |
| | GAQPEDEAEYYCALWYSNHWVFGGGTKLTVLGQPKAAPS | |
| | VTLFPPSSEELQANKATLVCLISDFYPGAVTVAWKADSS | |
| | PVKAGVETTTPSKQSNNKYAASSYLSLTPEQWKSHRSYS | |
| | CQVTHEGSTVEKTVAPTECS | |

Anti-P329G (VH1VL3) huIgG1

| | | |
|---|---|---|
| HCDR1 | RYWMN | 1 |
| HCDR2 | EITPDSSTINYTPSLKG | 40 |
| HCDR3 | PYDYGAWFAS | 3 |
| LCDR1 | GSSTGAVTTSNYAN | 52 |
| LCDR2 | GTNKRAP | 5 |
| LCDR3 | ALWYSNHWV | 6 |
| VH | EVQLVESGGGLVQPGGSLRLSCAASGFDFSRYWMNWVRQ | 41 |
| | APGKGLEWVGEITPDSSTINYTPSLKGRFTISRDNAKNS | |
| | LYLQMNSLRAEDTAVYYCVRPYDYGAWFASWGQGTLVTV | |
| | SS | |
| VL | QAVVTQEPSLTVSPGGTVTLTCGSSTGAVTTSNYANWFQ | 53 |
| | QKPGQAPRTLIGGTNKRAPGTPARFSGSLLGGKAALTLS | |
| | GAQPEDEAEYYCALWYSNHWVFGGGTKLTVL | |
| HC | EVQLVESGGGLVQPGGSLRLSCAASGFDFSRYWMNWVRQ | 42 |
| | APGKGLEWVGEITPDSSTINYTPSLKGRFTISRDNAKNS | |
| | LYLQMNSLRAEDTAVYYCVRPYDYGAWFASWGQGTLVTV | |
| | SSASTKGPSVFPLAPSSKSTSGGTAALGCLVKDYFPEPV | |
| | TVSWNSGALTSGVHTFPAVLQSSGLYSLSSVVTVPSSSL | |
| | GTQTYICNVNHKPSNTKVDKKVEPKSCDKTHTCPPCPAP | |
| | ELLGGPSVFLFPPKPKDTLMISRTPEVTCVVVDVSHEDP | |
| | EVKFNWYVDGVEVHNAKTKPREEQYNSTYRVVSVLTVLH | |
| | QDWLNGKEYKCKVSNKALPAPIEKTISKAKGQPREPQVY | |
| | TLPPSRDELTKNQVSLTCLVKGFYPSDIAVEWESNGQPE | |
| | NNYKTTPPVLDSDGSFFLYSKLTVDKSRWQQGNVFSCSV | |
| | MHEALHNHYTQKSLSLSP | |
| LC | QAVVTQEPSLTVSPGGTVTLTCGSSTGAVTTSNYANWFQ | 54 |
| | QKPGQAPRTLIGGTNKRAPGTPARFSGSLLGGKAALTLS | |
| | GAQPEDEAEYYCALWYSNHWVFGGGTKLTVLGQPKAAPS | |
| | VTLFPPSSEELQANKATLVCLISDFYPGAVTVAWKADSS | |
| | PVKAGVETTTPSKQSNNKYAASSYLSLTPEQWKSHRSYS | |
| | CQVTHEGSTVEKTVAPTECS | |

TABLE 7?
P329G IgG1 Fc variant

| | | |
|---|---|---|
| huIgG1 | EPKSCDKTHTCPPCPAPEAAGGPSVFLFPPKPKDTLMI | 55 |
| Fc | SRTPEVTCVVVDVSHEDPEVKFNWYVDGVEVHNAKTKP | |
| P329G | REEQYNSTYRVVSVLTVLHQDWLNGKEYKCKVSNKALG | |
| | APIEKTISKAKGQPREPQVCTLPPSRDELTKNQVSLSC | |
| | AVKGFYPSDIAVEWESNGQPENNYKTTPPVLDSDGSFF | |
| | LVSKLTVDKSRWQQGNVFSCSVMHEALHNHYTQKSLSL | |
| | SP | |

TABLE 8

| | | SEQ |
|---|---|---|
| Con- | | ID |
| struct | Amino acid sequence | NO |
| Human | ATGGCGCGCCCGCATCCGTGGTGGCTGTGCGTGCTGG | 56 |
| CD27 | GCACCCTGGTGGGCCTGAGCGCGACCCCGGCGCCGAA | |
| | AAGCTGCCCGGAACGCCATTATTGGGCGCAGGGCAAA | |
| | CTGTGCTGCCAGATGTGCGAACCGGGCACCTTTCTGG | |
| | TGAAAGATTGCGATCAGCATCGCAAAGCGGCGCAGTG | |
| | CGATCCGTGCATTCCGGGCGTGAGCTTTAGCCCGGAT | |
| | CATCATACCCGCCCGCATTGCGAAAGCTGCCGCCATT | |
| | GCAACAGCGGCCTGCTGGTGCGCAACTGCACCATTAC | |
| | CGCGAACGCGGAATGCGCGTGCCGCAACGGCTGGCAG | |
| | TGCCGCGATAAAGAATGCACCGAATGCGATCCGCTGC | |
| | CGAACCCGAGCCTGACCGCGCGCAGCAGCCAGGCGCT | |
| | GAGCCCGCATCCGCAGCCGACCCATCTGCCGTATGTG | |
| | AGCGAAATGCTGGAAGCGCGCACCGCGGGCCATATGC | |
| | AGACCCTGGCGGATTTTCGCCAGCTGCCGGCGCGCAC | |
| | CCTGAGCACCCATTGGCCGCCGCAGCGCAGCCTGTGC | |
| | AGCAGCGATTTTATTCGCATTCTGGTGATTTTTAGCG | |
| | GCATGTTTCTGGTGTTTACCCTGGCGGGCGCGCTGTT | |
| | TCTGCATCAGCGCCGCAAATATCGCAGCAACAAAGGC | |
| | GAAAGCCCGGTGGAACCGGCGGAACCGTGCCATTATA | |
| | GCTGCCCGCGCGAAGAAGAAGGCAGCACCATTCCGAT | |
| | TCAGGAAGATTATCGCAAACCGGAACCGGCGTGCAGC | |
| | CCG | |
| Human | MARPHPWWLCVLGTLVGLSATPAPKSCPERHYWAQGK | 57 |
| CD27 | LCCQMCEPGTFLVKDCDQHRKAAQCDPCIPGVSFSPD | |
| | HHTRPHCESCRHCNSGLLVRNCTITANAECACRNGWQ | |
| | CRDKECTECDPLPNPSLTARSSQALSPHPQPTHLPYV | |
| | SEMLEARTAGHMQTLADFRQLPARTLSTHWPPQRSLC | |
| | SSDFIRILVIFSGMFLVFTLAGALFLHQRRKYRSNKG | |
| | ESPVEPAEPCHYSCPREEEGSTIPIQEDYRKPEPACS | |
| | P | |
| Murine | ATGGCGTGGCCGCCGCCGTATTGGCTGTGCATGCTGG | 58 |
| CD27 | GCACCCTGGTGGGCCTGAGCGCGACCCTGGCGCCGAA | |
| | CAGCTGCCCGGATAAACATTATTGGACCGGCGGCGGC | |
| | CTGTGCTGCCGCATGTGCGAACCGGGCACCTTTTTTG | |
| | TGAAAGATTGCGAACAGGATCGCACCGCGGCGCAGTG | |
| | CGATCCGTGCATTCCGGGCACCAGCTTTAGCCCGGAT | |
| | TATCATACCCGCCCGCATTGCGAAAGCTGCCGCCATT | |
| | GCAACAGCGGCTTTCTGATTCGCAACTGCACCGTGAC | |
| | CGCGAACGCGGAATGCAGCTGCAGCAAAAACTGGCAG | |
| | TGCCGCGATCAGGAATGCACCGAATGCGATCCGCCGC | |
| | TGAACCCGGCGCTGACCCGCCAGCCGAGCGAAACCCC | |
| | GAGCCCGCAGCCGCCGCCGACCCATCTGCCGCATGGC | |
| | ACCGAAAAACCGAGCTGGCCGCTGCATCGCCAGCTGC | |
| | CGAACAGCACCGTGTATAGCCAGCGCAGCAGCCATCG | |
| | CCCGCTGTGCAGCAGCGATTGCATTCGCATTTTTGTG | |
| | ACCTTTAGCAGCATGTTTCTGATTTTTGTGCTGGGCG | |
| | CGATTCTGTTTTTTCATCAGCGCCGCAACCATGGCCC | |
| | GAACGAAGATCGCCAGGCGGTGCCGGAAGAACCGTGC | |
| | CCGTATAGCTGCCCGCGCGAAGAAGAAGGCAGCGCGA | |
| | TTCCGATTCAGGAAGATTATCGCAAACCGGAACCGGC | |
| | GTTTTATCCG | |
| Murine | MAWPPPYWLCMLGTLVGLSATLAPNSCPDKHYWTGGG | 59 |
| CD27 | LCCRMCEPGTFFVKDCEQDRTAAQCDPCIPGTSFSPD | |
| | YHTRPHCESCRHCNSGFLIRNCTVTANAECSCSKNWQ | |
| | CRDQECTECDPPLNPALTRQPSETPSPQPPPTHLPHG | |
| | TEKPSWPLHRQLPNSTVYSQRSSHRPLCSSDCIRIFV | |
| | TFSSMFLIFVLGAILFFHQRRNHGPNEDRQAVPEEPC | |
| | PYSCPREEEGSAIPIQEDYRKPEPAFYP | |
| Human | ATGCTGCGCCTGCTGCTGGCGCTGAACCTGTTTCCGA | 60 |
| CD28 | GCATTCAGGTGACCGGCAACAAAATTCTGGTGAAACA | |
| | GAGCCCGATGCTGGTGGCGTATGATAACGCGGTGAAC | |
| | CTGAGCTGCAAATATAGCTATAACCTGTTTAGCCGCG | |
| | AATTTCGCGCGAGCCTGCATAAAGGCCTGGATAGCGC | |
| | GGTGGAAGTGTGCGTGGTGTATGGCAACTATAGCCAG | |
| | CAGCTGCAGGTGTATAGCAAAACCGGCTTTAACTGCG | |
| | ATGGCAAACTGGGCAACGAAAGCGTGACCTTTTATCT | |
| | GCAGAACCTGTATGTGAACCAGACCGATATTTATTTT | |
| | TGCAAAATTGAAGTGATGTATCCGCCGCCGTATCTGG | |
| | ATAACGAAAAAAGCAACGGCACCATTATTCATGTGAA | |
| | AGGCAAACATCTGTGCCCGAGCCCGCTGTTTCCGGGC | |
| | CCGAGCAAACCGTTTTGGGTGCTGGTGGTGGTGGGCG | |
| | GCGTGCTGGCGTGCTATAGCCTGCTGGTGACCGTGGC | |
| | GTTTATTATTTTTTGGGTGCGCAGCAAACGCAGCCGC | |
| | CTGCTGCATAGCGATTATATGAACATGACCCCGCGCC | |
| | GCCCGGGCCCGACCCGCAAACATTATCAGCCGTATGC | |
| | GCCGCCGCGCGATTTTGCGGCGTATCGCAGC | |
| Human | MLRLLLALNLFPSIQVTGNKILVKQSPMLVAYDNAVN | 61 |
| CD28 | LSCKYSYNLFSREFRASLHKGLDSAVEVCVVYGNYSQ | |
| | QLQVYSKTGFNCDGKLGNESVTFYLQNLYVNQTDIYF | |
| | CKIEVMYPPPYLDNEKSNGTIIHVKGKHLCPSPLFPG | |
| | PSKPFWVLVVVGGVLACYSLLVTVAFIIFWVRSKRSR | |
| | LLHSDYMNMTPRRPGPTRKHYQPYAPPRDFAAYRS | |
| Murine | ATGACCCTGCGCCTGCTGTTTCTGGCGCTGAACTTTT | 62 |
| CD28 | TTAGCGTGCAGGTGACCGAAAACAAAATTCTGGTGAA | |
| | ACAGAGCCCGCTGCTGGTGGTGGATAGCAACGAAGTG | |
| | AGCCTGAGCTGCCGCTATAGCTATAACCTGCTGGCGA | |
| | AAGAATTTCGCGCGAGCCTGTATAAAGGCGTGAACAG | |
| | CGATGTGGAAGTGTGCGTGGGCAACGGCAACTTTACC | |
| | TATCAGCCGCAGTTTCGCAGCAACGCGGAATTTAACT | |
| | GCGATGGCGATTTTGATAACGAAACCGTGACCTTTCG | |
| | CCTGTGGAACCTGCATGTGAACCATACCGATATTTAT | |
| | TTTTGCAAAATTGAATTTATGTATCCGCCGCCGTATC | |
| | TGGATAACGAACGCAGCAACGGCACCATTATTCATAT | |
| | TAAAGAAAAACATCTGTGCCATACCCAGAGCAGCCCG | |
| | AAACTGTTTTGGGCGCTGGTGGTGGTGGCGGGCGTGC | |
| | TGTTTTGCTATGGCCTGCTGGTGACCGTGGCGCTGTG | |
| | CGTGATTTGGACCAACAGCCGCCGCAACCGCCTGCTG | |
| | CAGAGCGATTATATGAACATGACCCCGCGCCGCCCGG | |
| | GCCTGACCCGCAAACCGTATCAGCCGTATGCGCCGGC | |
| | GCGCGATTTTGCGGCGTATCGCCCG | |
| Murine | MTLRLLFLALNFFSVQVTENKILVKQSPLLVVDSNEV | 63 |
| CD28 | SLSCRYSYNLLAKEFRASLYKGVNSDVEVCVGNGNFT | |
| | YQPQFRSNAEFNCDGDFDNETVTFRLWNLHVNHTDIY | |
| | FCKIEFMYPPPYLDNERSNGTIIHIKEKHLCHTQSSP | |
| | KLFWALVVVAGVLFCYGLLVTVALCVIWTNSRRNRLL | |
| | QSDYMNMTPRRPGLTRKPYQPYAPARDFAAYRP | |
| Human | ATGGGAAACAGCTGTTACAACATAGTAGCCACTCTGT | 64 |
| CD137 | TGCTGGTCCTCAACTTTGAGAGGACAAGATCATTGCA | |
| | GGATCCTTGTAGTAACTGCCCAGCTGGTACATTCTGT | |
| | GATAATAACAGGAATCAGATTTGCAGTCCCTGTCCTC | |
| | CAAATAGTTTCTCCAGCGCAGGTGGACAAAGGACCTG | |
| | TGACATATGCAGGCAGTGTAAAGGTGTTTTCAGGACC | |
| | AGGAAGGAGTGTTCCTCCACCAGCAATGCAGAGTGTG | |
| | ACTGCACTCCAGGGTTTCACTGCCTGGGGGCAGGATG | |
| | CAGCATGTGTGAACAGGATTGTAAACAAGGTCAAGAA | |
| | CTGACAAAAAAAGGTTGTAAAGACTGTTGCTTTGGGA | |
| | CATTTAACGATCAGAAACGTGGCATCTGTCGACCCTG | |
| | GACAAACTGTTCTTTGGATGGAAAGTCTGTGCTTGTG | |
| | AATGGGACGAAGGAGAGGGACGTGGTCTGTGGACCAT | |
| | CTCCAGCCGACCTCTCTCCGGGAGCATCCTCTGTGAC | |
| | CCCGCCTGCCCCTGCGAGAGAGCCAGGACACTCTCCG | |
| | CAGATCATCTCCTTCTTTCTTGCGCTGACGTCGACTG | |
| | CGTTGCTCTTCCTGCTGTTCTTCCTCACGCTCCGTTT | |
| | CTCTGTTGTTAAACGGGGCAGAAAGAAACTCCTGTAT | |
| | ATATTCAAACAACCATTTATGAGACCAGTACAAACTA | |
| | CTCAAGAGGAAGATGGCTGTAGCTGCCGATTTCCAGA | |
| | AGAAGAAGAAGGAGGATGTGAACTGTGA | |
| Human | MGNSCYNIVATLLLVLNFERTRSLQDPCSNCPAGTFC | 65 |
| CD137 | DNNRNQICSPCPPNSFSSAGGQRTCDICRQCKGVFRT | |
| | RKECSSTSNAECDCTPGFHCLGAGCSMCEQDCKQGQE | |
| | LTKKGCKDCCFGTFNDQKRGICRPWTNCSLDGKSVLV | |
| | NGTKERDVVCGPSPADLSPGASSVTPPAPAREPGHSP | |
| | QIISFFLALTSTALLFLLFFLTLRFSVVKRGRKKLLY | |
| | IFKQPFMRPVQTTQEEDGCSCRFPEEEEGGCEL | |
| Murine | ATGGGCAACAACTGCTATAACGTGGTGGTGATTGTGC | 66 |
| CD137 | TGCTGCTGGTGGGCTGCGAAAAAGTGGGCGCGGTGCA | |
| | GAACAGCTGCGATAACTGCCAGCCGGGCACCTTTTGC | |
| | CGCAAATATAACCCGGTGTGCAAAAGCTGCCCGCCGA | |
| | GCACCTTTAGCAGCATTGGCGGCCAGCCGAACTGCAA | |
| | CATTTGCCGCGTGTGCGCGGGCTATTTTCGCTTTAAA | |
| | AAATTTTGCAGCAGCACCCATAACGCGGAATGCGAAT | |
| | GCATTGAAGGCTTTCATTGCCTGGGCCCGCAGTGCAC | |
| | CCGCTGCGAAAAAGATTGCCGCCCGGGCCAGGAACTG | |
| | ACCAAACAGGGCTGCAAAACCTGCAGCCTGGGCACCT | |
| | TTAACGATCAGAACGGCACCGGCGTGTGCCGCCCGTG | |
| | GACCAACTGCAGCCTGGATGGCCGCAGCGTGCTGAAA | |
| | ACCGGCACCACCGAAAAAGATGTGGTGTGCGGCCCGC | |
| | CGGTGGTGAGCTTTAGCCCGAGCACCACCATTAGCGT | |
| | GACCCCGGAAGGCGGCCCGGGCGGCCATAGCCTGCAG | |
| | GTGCTGACCCTGTTTCTGGCGCTGACCAGCGCGCTGC | |
| | TGCTGGCGCTGATTTTTATTACCCTGCTGTTTAGCGT | |
| | GCTGAAATGGATTCGCAAAAAATTTCCGCATATTTTT | |
| | AAACAGCCGTTTAAAAAAACCACCGGCGCGGCGCAGG | |
| | AAGAAGATGCGTGCAGCTGCCGCTGCCCGCAGGAAGA | |
| | AGAAGGCGGCGGCGGCGGCTATGAACTG | |
| Murine | MGNNCYNVVVIVLLLVGCEKVGAVQNSCDNCQPGTFC | 67 |
| CD137 | RKYNPVCKSCPPSTFSSIGGQPNCNICRVCAGYFRFK | |
| | KFCSSTHNAECECIEGFHCLGPQCTRCEKDCRPGQEL | |
| | TKQGCKTCSLGTFNDQNGTGVCRPWTNCSLDGRSVLK | |
| | TGTTEKDVVCGPPVVSFSPSTTISVTPEGGPGGHSLQ | |
| | VLTLFLALTSALLLALIFITLLFSVLKWIRKKFPHIF | |
| | KQPFKKTTGAAQEEDACSCRCPQEEEGGGGGYEL | |
| Human | ATGTGCGTGGGCGCGCGCCGCCTGGGCCGCGGCCCGT | 68 |
| OX40 | GCGCGGCGCTGCTGCTGCTGGGCCTGGGCCTGAGCAC | |
| | CGTGACCGGCCTGCATTGCGTGGGCGATACCTATCCG | |
| | AGCAACGATCGCTGCTGCCATGAATGCCGCCCGGGCA | |
| | ACGGCATGGTGAGCCGCTGCAGCCGCAGCCAGAACAC | |
| | CGTGTGCCGCCCGTGCGGCCCGGGCTTTTATAACGAT | |
| | GTGGTGAGCAGCAAACCGTGCAAACCGTGCACCTGGT | |
| | GCAACCTGCGCAGCGGCAGCGAACGCAAACAGCTGTG | |
| | CACCGCGACCCAGGATACCGTGTGCCGCTGCCGCGCG | |
| | GGCACCCAGCCGCTGGATAGCTATAAACCGGGCGTGG | |
| | ATTGCGCGCCGTGCCCGCCGGGCCATTTTAGCCCGGG | |
| | CGATAACCAGGCGTGCAAACCGTGGACCAACTGCACC | |
| | CTGGCGGGCAAACATACCCTGCAGCCGGCGAGCAACA | |
| | GCAGCGATGCGATTTGCGAAGATCGCGATCCGCCGGC | |
| | GACCCAGCCGCAGGAAACCCAGGGCCCGCCGGCGCGC | |
| | CCGATTACCGTGCAGCCGACCGAAGCGTGGCCGCGCA | |
| | CCAGCCAGGGCCCGAGCACCCGCCCGGTGGAAGTGCC | |
| | GGGCGGCCGCGCGGTGGCGGCGATTCTGGGCCTGGGC | |
| | CTGGTGCTGGGCCTGCTGGGCCCGCTGGCGATTCTGC | |
| | TGGCGCTGTATCTGCTGCGCCGCGATCAGCGCCTGCC | |
| | GCCGGATGCGCATAAACCGCCGGGCGGCGGCAGCTTT | |
| | CGCACCCCGATTCAGGAAGAACAGGCGGATGCGCATA | |
| | GCACCCTGGCGAAAATT | |
| Human | MCVGARRLGRGPCAALLLLGLGLSTVTGLHCVGDTYP | 69 |
| OX40 | SNDRCCHECRPGNGMVSRCSRSQNTVCRPCGPGFYND | |
| | VVSSKPCKPCTWCNLRSGSERKQLCTATQDTVCRCRA | |
| | GTQPLDSYKPGVDCAPCPPGHFSPGDNQACKPWTNCT | |
| | LAGKHTLQPASNSSDAICEDRDPPATQPQETQGPPAR | |
| | PITVQPTEAWPRTSQGPSTRPVEVPGGRAVAAILGLG | |
| | LVLGLLGPLAILLALYLLRRDQRLPPDAHKPPGGGSF | |
| | RTPIQEEQADAHSTLAKI | |
| Murine | ATGTATGTGTGGGTGCAGCAGCCGACCGCGCTGCTGC | 70 |
| OX40 | TGCTGGCGCTGACCCTGGGCGTGACCGCGCGCCGCCT | |
| | GAACTGCGTGAAACATACCTATCCGAGCGGCCATAAA | |
| | TGCTGCCGCGAATGCCAGCCGGGCCATGGCATGGTGA | |
| | GCCGCTGCGATCATACCCGCGATACCCTGTGCCATCC | |
| | GTGCGAAACCGGCTTTTATAACGAAGCGGTGAACTAT | |
| | GATACCTGCAAACAGTGCACCCAGTGCAACCATCGCA | |
| | GCGGCAGCGAACTGAAACAGAACTGCACCCCGACCCA | |
| | GGATACCGTGTGCCGCTGCCGCCCGGGCACCCAGCCG | |
| | CGCCAGGATAGCGGCTATAAACTGGGCGTGGATTGCG | |
| | TGCCGTGCCCGCCGGGCCATTTTAGCCCGGGCAACAA | |
| | CCAGGCGTGCAAACCGTGGACCAACTGCACCCTGAGC | |
| | GGCAAACAGACCCGCCATCCGGCGAGCGATAGCCTGG | |
| | ATGCGGTGTGCGAAGATCGCAGCCTGCTGGCGACCCT | |
| | GCTGTGGGAAACCCAGCGCCCGACCTTTCGCCCGACC | |
| | ACCGTGCAGAGCACCACCGTGTGGCCGCGCACCAGCG | |
| | AACTGCCGAGCCCGCCGACCCTGGTGACCCCGGAAGG | |
| | CCCGGCGTTTGCGGTGCTGCTGGGCCTGGGCCTGGGC | |
| | CTGCTGGCGCCGCTGACCGTGCTGCTGGCGCTGTATC | |
| | TGCTGCGCAAAGCGTGGCGCCTGCCGAACACCCCGAA | |
| | ACCGTGCTGGGGCAACAGCTTTCGCACCCCGATTCAG | |
| | GAAGAACATACCGATGCGCATTTTACCCTGGCGAAAA | |
| | TT | |
| Murine | MYVWVQQPTALLLLALTLGVTARRLNCVKHTYPSGHK | 71 |
| OX40 | CCRECQPGHGMVSRCDHTRDTLCHPCETGFYNEAVNY | |
| | DTCKQCTQCNHRSGSELKQNCTPTQDTVCRCRPGTQP | |
| | RQDSGYKLGVDCVPCPPGHFSPGNNQACKPWTNCTLS | |
| | GKQTRHPASDSLDAVCEDRSLLATLLWETQRPTFRPT | |
| | TVQSTTVWPRTSELPSPPTLVTPEGPAFAVLLGLGLG | |
| | LLAPLTVLLALYLLRKAWRLPNTPKPCWGNSFRTPIQ | |
| | EEHTDAHFTLAKI | |
| Human | ATGAAAAGCGGCCTGTGGTATTTTTTTCTGTTTTGCC | 72 |
| ICOS | TGCGCATTAAAGTGCTGACCGGCGAAATTAACGGCAG | |
| | CGCGAACTATGAAATGTTTATTTTTCATAACGGCGGC | |
| | GTGCAGATTCTGTGCAAATATCCGGATATTGTGCAGC | |
| | AGTTTAAAATGCAGCTGCTGAAAGGCGGCCAGATTCT | |
| | GTGCGATCTGACCAAAACCAAAGGCAGCGGCAACACC | |
| | GTGAGCATTAAAAGCCTGAAATTTTGCCATAGCCAGC | |
| | TGAGCAACAACAGCGTGAGCTTTTTTCTGTATAACCT | |
| | GGATCATAGCCATGCGAACTATTATTTTTGCAACCTG | |
| | AGCATTTTTGATCCGCCGCCGTTTAAAGTGACCCTGA | |
| | CCGGCGGCTATCTGCATATTTATGAAAGCCAGCTGTG | |
| | CTGCCAGCTGAAATTTTGGCTGCCGATTGGCTGCGCG | |
| | GCGTTTGTGGTGGTGTGCATTCTGGGCTGCATTCTGA | |
| | TTTGCTGGCTGACCAAAAAAAAATATAGCAGCAGCGT | |
| | GCATGATCCGAACGGCGAATATATGTTTATGCGCGCG | |
| | GTGAACACCGCGAAAAAAAGCCGCCTGACCGATGTGA | |
| | CCCTG | |
| Human | MKSGLWYFFLFCLRIKVLTGEINGSANYEMFIFHNGG | 73 |
| ICOS | VQILCKYPDIVQQFKMQLLKGGQILCDLTKTKGSGNT | |
| | VSIKSLKFCHSQLSNNSVSFFLYNLDHSHANYYFCNL | |
| | SIFDPPPFKVTLTGGYLHIYESQLCCQLKFWLPIGCA | |
| | AFVVVCILGCILICWLTKKKYSSSVHDPNGEYMFMRA | |
| | VNTAKKSRLTDVTL | |
| Murine | ATGAAACCGTATTTTTGCCGCGTGTTTGTGTTTTGCT | 74 |
| ICOS | TTCTGATTCGCCTGCTGACCGGCGAAATTAACGGCAG | |
| | CGCGGATCATCGCATGTTTAGCTTTCATAACGGCGGC | |
| | GTGCAGATTAGCTGCAAATATCCGGAAACCGTGCAGC | |
| | AGCTGAAAATGCGCCTGTTTCGCGAACGCGAAGTGCT | |
| | GTGCGAACTGACCAAAACCAAAGGCAGCGGCAACGCG | |
| | GTGAGCATTAAAAACCCGATGCTGTGCCTGTATCATC | |
| | TGAGCAACAACAGCGTGAGCTTTTTTCTGAACAACCC | |
| | GGATAGCAGCCAGGGCAGCTATTATTTTTGCAGCCTG | |
| | AGCATTTTTGATCCGCCGCCGTTTCAGGAACGCAACC | |
| | TGAGCGGCGGCTATCTGCATATTTATGAAAGCCAGCT | |
| | GTGCTGCCAGCTGAAACTGTGGCTGCCGGTGGGCTGC | |
| | GCGGCGTTTGTGGTGGTGCTGCTGTTTGGCTGCATTC | |
| | TGATTATTTGGTTTAGCAAAAAAAAATATGGCAGCAG | |
| | CGTGCATGATCCGAACAGCGAATATATGTTTATGGCG | |
| | GCGGTGAACACCAACAAAAAAAGCCGCCTGGCGGGCG | |
| | TGACCAGC | |
| Murine | MKPYFCRVFVFCFLIRLLTGEINGSADHRMFSFHNGG | 75 |
| ICOS | VQISCKYPETVQQLKMRLFREREVLCELTKTKGSGNA | |
| | VSIKNPMLCLYHLSNNSVSFFLNNPDSSQGSYYFCSL | |
| | SIFDPPPFQERNLSGGYLHIYESQLCCQLKLWLPVGC | |
| | AAFVVVLLFGCILIIWFSKKKYGSSVHDPNSEYMFMA | |
| | AVNTNKKSRLAGVTS | |
| Human | ATGATTCATCTGGGCCATATTCTGTTTCTGCTGCTGC | 76 |
| DAP10 | TGCCGGTGGCGGCGGCGCAGACCACCCCGGGCGAACG | |
| | CAGCAGCCTGCCGGCGTTTTATCCGGGCACCAGCGGC | |
| | AGCTGCAGCGGCTGCGGCAGCCTGAGCCTGCCGCTGC | |
| | TGGCGGGCCTGGTGGCGGCGGATGCGGTGGCGAGCCT | |
| | GCTGATTGTGGGCGCGGTGTTTCTGTGCGCGCGCCCG | |
| | CGCCGCAGCCCGGCGCAGGAAGATGGCAAAGTGTATA | |
| | TTAACATGCCGGGCCGCGGC | |
| Human | MIHLGHILFLLLLPVAAAQTTPGERSSLPAFYPGTSG | 77 |
| DAP10 | SCSGCGSLSLPLLAGLVAADAVASLLIVGAVFLCARP | |
| | RRSPAQEDGKVYINMPGRG | |
| Murine | ATGGATCCGCCGGGCTATCTGCTGTTTCTGCTGCTGC | 78 |
| DAP10 | TGCCGGTGGCGGCGAGCCAGACCAGCGCGGGCAGCTG | |
| | CAGCGGCTGCGGCACCCTGAGCCTGCCGCTGCTGGCG | |
| | GGCCTGGTGGCGGCGGATGCGGTGATGAGCCTGCTGA | |
| | TTGTGGGCGTGGTGTTTGTGTGCATGCGCCCGCATGG | |
| | CCGCCCGGCGCAGGAAGATGGCCGCGTGTATATTAAC | |
| | ATGCCGGGCCGCGGC | |
| Murine | MDPPGYLLFLLLLPVAASQTSAGSCSGCGTLSLPLLA | 79 |
| DAP10 | GLVAADAVMSLLIVGVVFVCMRPHGRPAQEDGRVYIN | |
| | MPGRG | |
| Human | ATGGGGGGACTTGAACCCTGCAGCAGGCTCCTGCTCC | 80 |
| DAP12 | TGCCTCTCCTGCTGGCTGTAAGTGGTCTCCGTCCTGT | |
| | CCAGGCCCAGGCCCAGAGCGATTGCAGTTGCTCTACG | |
| | GTGAGCCCGGGCGTGCTGGCAGGGATCGTGATGGGAG | |
| | ACCTGGTGCTGACAGTGCTCATTGCCCTGGCCGTGTA | |
| | CTTCCTGGGCCGGCTGGTCCCTCGGGGGCGAGGGGCT | |
| | GCGGAGGCAGCGACCCGGAAACAGCGTATCACTGAGA | |
| | CCGAGTCGCCTTATCAGGAGCTCCAGGGTCAGAGGTC | |
| | GGATGTCTACAGCGACCTCAACACACAGAGGCCGTAT | |
| | TACAAATGA | |
| Human | MGGLEPCSRLLLLPLLLAVSGLRPVQAQAQSDCSCST | 81 |
| DAP12 | VSPGVLAGIVMGDLVLTVLIALAVYFLGRLVPRGRGA | |
| | AEAATRKQRITETESPYQELQGQRSDVYSDLNTQRPY | |
| | YK | |
| Murine | ATGGGGGCTCTGGAGCCCTCCTGGTGCCTTCTGTTCC | 82 |
| DAP12 | TTCCTGTCCTCCTGACTGTGGGAGGATTAAGTCCCGT | |
| | ACAGGCCCAGAGTGACACTTTCCCAAGATGCGACTGT | |
| | TCTTCCGTGAGCCCTGGTGTACTGGCTGGGATTGTTC | |
| | TGGGTGACTTGGTGTTGACTCTGCTGATTGCCCTGGC | |
| | TGTGTACTCTCTGGGCCGCCTGGTCTCCCGAGGTCAA | |
| | GGGACAGCGGAAGGGACCCGGAAACAACACATTGCTG | |
| | AGACTGAGTCGCCTTATCAGGAGCTTCAGGGTCAGAG | |
| | ACCAGAAGTATACAGTGACCTCAACACACAGAGGCAA | |
| | TATTACAGATGA | |
| Murine | MGALEPSWCLLFLPVLLTVGGLSPVQAQSDTFPRCDC | 83 |
| DAP12 | SSVSPGVLAGIVLGDLVLTLLIALAVYSLGRLVSRGQ | |
| | GTAEGTRKQHIAETESPYQELQGQRPEVYSDLNTQRQ | |
| | YYR | |
| Human | MKWKALFTAAILQAQLPITEAQSFGLLDPKLCYLLDG | 84 |
| CD3z | ILFIYGVILTALFLRVKFSRSADAPAYQQGQNQLYNE | |
| | LNLGRREEYDVLDKRRGRDPEMGGKPQRRKNPQEGLY | |
| | NELQKDKMAEAYSEIGMKGERRRGKGHDGLYQGLSTA | |
| | TKDTYDALHMQALPPR | |
| Human | ATGAAGTGGAAGGCGCTTTTCACCGCGGCCATCCTGC | 85 |
| CD3z | AGGCACAGTTGCCGATTACAGAGGCACAGAGCTTTGG | |
| | CCTGCTGGATCCCAAACTCTGCTACCTGCTGGATGGA | |
| | ATCCTCTTCATCTATGGTGTCATTCTCACTGCCTTGT | |
| | TCCTGAGAGTGAAGTTCAGCAGGAGCGCAGAGCCCCC | |
| | CGCGTACCAGCAGGGCCAGAACCAGCTCTATAACGAG | |
| | CTCAATCTAGGACGAAGAGAGGAGTACGATGTTTTGG | |
| | ACAAGAGACGTGGCCGGGACCCTGAGATGGGGGGAAA | |
| | GCCGAGAAGGAAGAACCCTCAGGAAGGCCTGTACAAT | |
| | GAACTGCAGAAAGATAAGATGGCGGAGGCCTACAGTG | |
| | AGATTGGGATGAAAGGCGAGCGCCGGAGGGGCAAGGG | |
| | GCACGATGGCCTTTACCAGGGTCTCAGTACAGCCACC | |
| | AAGGACACCTACGACGCCCTTCACATGCAGGCCCTGC | |
| | CCCCTCGCTAA | |
| Murine | MKWKVSVLACILHVRFPGAEAQSFGLLDPKLCYLLDG | 86 |
| CD3z | ILFIYGVIITALYLRAKFSRSAETAANLQDPNQLYNE | |
| | LNLGRREEYDVLEKKRARDPEMGGKQQRRRNPQEGVY | |
| | NALQKDKMAEAYSEIGTKGERRRGKGHDGLYQGLSTA | |
| | TKDTYDALHMQTLAPR | |
| Murine | ATGAAGTGGAAAGTGTCTGTTCTCGCCTGCATCCTCC | 87 |
| CD3z | ACGTGCGGTTCCCAGGAGCAGAGGCACAGAGCTTTGG | |
| | TCTGCTGGATCCCAAACTCTGCTACTTGCTAGATGGA | |
| | ATCCTCTTCATCTACGGAGTCATCATCACAGCCCTGT | |
| | ACCTGAGAGCAAAATTCAGCAGGAGTGCAGAGACTGC | |
| | TGCCAACCTGCAGGACCCCAACCAGCTCTACAATGAG | |
| | CTCAATCTAGGGCGAAGAGAGGAATATGACGTCTTGG | |
| | AGAAGAAGCGGGCTCGGGATCCAGAGATGGGAGGCAA | |
| | ACAGCAGAGGAGGAGGAACCCCCAGGAAGGCGTATAC | |
| | AATGCACTGCAGAAAGACAAGATGGCAGAAGCCTACA | |
| | GTGAGATCGGCACAAAAGGCGAGAGGCGGAGAGGCAA | |
| | GGGGCACGATGGCCTTTACCAGGGTCTCAGCACTGCC | |
| | ACCAAGGACACCTATGATGCCCTGCATATGCAGACCC | |
| | TGGCCCCTCGCTAA | |
| Human | MWQLLLPTALLLLVSAGMRTEDLPKAVVFLEPQWYRV | 88 |
| FCGR3A | LEKDSVTLKCQGAYSPEDNSTQWFHNESLISSQASSY | |
| | FIDAATVDDSGEYRCQTNLSTLSDPVQLEVHIGWLLL | |
| | QAPRWVFKEEDPIHLRCHSWKNTALHKVTYLQNGKGR | |
| | KYFHHNSDFYIPKATLKDSGSYFCRGLFGSKNVSSET | |
| | VNITITQGLAVSTISSFFPPGYQVSFCLVMVLLFAVD | |
| | TGLYFSVKTNIRSSTRDWKDHKFKWRKDPQDK | |
| Human | ATGTGGCAGCTGCTGCTGCCGACCGCGCTGCTGCTGC | 89 |
| FCGR3A | TGGTGAGCGCGGGCATGCGCACCGAAGATCTGCCGAA | |
| | AGCGGTGGTGTTTCTGGAACCGCAGTGGTATCGCGTG | |
| | CTGGAAAAAGATAGCGTGACCCTGAAATGCCAGGGCG | |
| | CGTATAGCCCGGAAGATAACAGCACCCAGTGGTTTCA | |
| | TAACGAAAGCCTGATTAGCAGCCAGGCGAGCAGCTAT | |
| | TTTATTGATGCGGCGACCGTGGATGATAGCGGCGAAT | |
| | ATCGCTGCCAGACCAACCTGAGCACCCTGAGCGATCC | |
| | GGTGCAGCTGGAAGTGCATATTGGCTGGCTGCTGCTG | |
| | CAGGCGCCGCGCTGGGTGTTTAAAGAAGAAGATCCGA | |
| | TTCATCTGCGCTGCCATAGCTGGAAAAACACCGCGCT | |
| | GCATAAAGTGACCTATCTGCAGAACGGCAAAGGCCGC | |
| | AAATATTTTCATCATAACAGCGATTTTTATATTCCGA | |
| | AAGCGACCCTGAAAGATAGCGGCAGCTATTTTTGCCG | |
| | CGGCCTGTTTGGCAGCAAAAACGTGAGCAGCGAAACC | |
| | GTGAACATTACCATTACCCAGGGCCTGGCGGTGAGCA | |
| | CCATTAGCAGCTTTTTTCCGCCGGGCTATCAGGTGAG | |
| | CTTTTGCCTGGTGATGGTGCTGCTGTTTGCGGTGGAT | |
| | ACCGGCCTGTATTTTAGCGTGAAAACCAACATTCGCA | |
| | GCAGCACCCGCGATTGGAAAGATCATAAATTTAAATG | |
| | GCGCAAAGATCCGCAGGATAAA | |
| Murine | MFQNAHSGSQWLLPPLTILLLFAFADRQSAALPKAVV | 90 |
| FCGR3A | KLDPPWIQVLKEDMVTLMCEGTHNPGNSSTQWFHNGR | |
| | SIRSQVQASYTFKATVNDSGEYRCQMEQTRLSDPVDL | |
| | GVISDWLLLQTPQRVFLEGETITLRCHSWRNKLLNRI | |
| | SFFHNEKSVRYHHYKSNFSIPKANHSHSGDYYCKGSL | |
| | GSTQHQSKPVTITVQDPATTSSISLVWYHTAFSLVMC | |
| | LLFAVDTGLYFYVRRNLQTPREYWRKSLSIRKHQAPQ | |
| | DK | |
| Murine | ATGTTTCAGAATGCACACTCTGGAAGCCAATGGCTAC | 91 |
| FCGR3A | TTCCACCACTGACAATTCTGCTGCTGTTTGCTTTTGC | |
| | AGACAGGCAGAGTGCAGCTCTTCCGAAGGCTGTGGTG | |
| | AAACTGGACCCCCCATGGATCCAGGTGCTCAAGGAAG | |
| | ACATGGTGACACTGATGTGCGAAGGGACCCACAACCC | |
| | TGGGAACTCTTCTACCCAGTGGTTCCACAACGGGAGG | |
| | TCCATCCGGAGCCAGGTCCAAGCCAGTTACACGTTTA | |
| | AGGCCACAGTCAATGACAGTGGAGAATATCGGTGTCA | |
| | AATGGAGCAGACCCGCCTCAGCGACCCTGTAGATCTG | |
| | GGAGTGATTTCTGACTGGCTGCTGCTCCAGACCCCTC | |
| | AGCGGGTGTTTCTGGAAGGGGAAACCATCACGCTAAG | |
| | GTGCCATAGCTGGAGGAACAAACTACTGAACAGGATC | |
| | TCATTCTTCCATAATGAAAAATCCGTGAGGTATCATC | |
| | ACTACAAAAGTAATTTCTCTATCCCAAAAGCCAACCA | |
| | CAGTCACAGTGGGGACTACTACTGCAAAGGAAGTCTA | |
| | GGAAGTACACAGCACCAGTCCAAGCCTGTCACCATCA | |
| | CTGTCCAAGATCCAGCAACTACATCCTCCATCTCTCT | |
| | AGTCTGGTACCACACTGCTTTCTCCCTAGTGATGTGC | |
| | CTCCTGTTTGCAGTGGACACGGGCCTTTATTTCTACG | |
| | TACGGAGAAATCTTCAAACCCCGAGGGAGTACTGGAG | |
| | GAAGTCCCTGTCAATCAGAAAGCACCAGGCTCCTCAA | |
| | GACAAGTGA | |
| Human | MGWIRGRRSRHSWEMSEFHNYNLDLKKSDFSTRWQKQ | 92 |
| NKG2D | RCPVVKSKCRENASPFFFCCFIAVAMGIRFIIMVAIW | |
| | SAVELNSLFNQEVQIPLTESYCGPCPKNWICYKNNCY | |
| | QFFDESKNWYESQASCMSQNASLLKVYSKEDQDLLKL | |
| | VKSYHWMGLVHIPTNGSWQWEDGSILSPNLLTIIEMQ | |
| | KGDCALYASSFKGYIENCSTPNTYICMQRTV | |
| Human | ATGGGCTGGATTCGCGGCCGCCGCAGCCGCCATAGCT | 93 |
| NKG2D | GGGAAATGAGCGAATTTCATAACTATAACCTGGATCT | |
| | GAAAAAAAGCGATTTTAGCACCCGCTGGCAGAAACAG | |
| | CGCTGCCCGGTGGTGAAAAGCAAATGCCGCGAAAACG | |
| | CGAGCCCGTTTTTTTTTTGCTGCTTTATTGCGGTGGC | |
| | GATGGGCATTCGCTTTATTATTATGGTGGCGATTTGG | |
| | AGCGCGGTGTTTCTGAACAGCCTGTTTAACCAGGAAG | |
| | TGCAGATTCCGCTGACCGAAAGCTATTGCGGCCCGTG | |
| | CCCGAAAAACTGGATTTGCTATAAAAACAACTGCTAT | |
| | CAGTTTTTTGATGAAAGCAAAAACTGGTATGAAAGCC | |
| | AGGCGAGCTGCATGAGCCAGAACGCGAGCCTGCTGAA | |
| | AGTGTATAGCAAAGAAGATCAGGATCTGCTGAAACTG | |
| | GTGAAAAGCTATCATTGGATGGGCCTGGTGCATATTC | |
| | CGACCAACGGCAGCTGGCAGTGGGAAGATGGCAGCAT | |
| | TCTGAGCCCGAACCTGCTGACCATTATTGAAATGCAG | |
| | AAAGGCGATTGCGCGCTGTATGCGAGCAGCTTTAAAG | |
| | GCTATATTGAAAACTGCAGCACCCCGAACACCTATAT | |
| | TTGCATGCAGCGCACCGTG | |
| Murine | MALIRDRKSHHSEMSKCHNYDLKPAKWDTSQEQQKQR | 94 |
| NKG2D | LALTTSQPGENGIIRGRYPIEKLKISPMFVVRVLAIA | |
| | LAIRFTLNTLMWLAIFKETFQPVLCNKEVPVSSREGY | |
| | CGPCPNNWICHRNNCYQFFNEEKTWNQSQASCLSQNS | |
| | SLLKIYSKEEQDFLKLVKSYHWMGLVQIPANGSWQWE | |
| | DGSSLSYNQLTLVEIPKGSCAVYGSSFKAYTEDCANL | |
| | NTYICMKRAV | |
| Murine | ATGGCGCTGATTCGCGATCGCAAAAGCCATCATAGCG | 95 |
| NKG2D | AAATGAGCAAATGCCATAACTATGATCTGAAACCGGC | |
| | GAAATGGGATACCAGCCAGGAACAGCAGAAACAGCGC | |
| | CTGGCGCTGACCACCAGCCAGCCGGGCGAAAACGGCA | |
| | TTATTCGCGGCCGCTATCCGATTGAAAAACTGAAAAT | |
| | TAGCCCGATGTTTGTGGTGCGCGTGCTGGCGATTGCG | |
| | CTGGCGATTCGCTTTACCCTGAACACCCTGATGTGGC | |
| | TGGCGATTTTTAAAGAAACCTTTCAGCCGGTGCTGTG | |
| | CAACAAAGAAGTGCCGGTGAGCAGCCGCGAAGGCTAT | |
| | TGCGGCCCGTGCCCGAACAACTGGATTTGCCATCGCA | |
| | ACAACTGCTATCAGTTTTTTAACGAAGAAAAAACCTG | |
| | GAACCAGAGCCAGGCGAGCTGCCTGAGCCAGAACAGC | |
| | AGCCTGCTGAAAATTTATAGCAAAGAAGAACAGGATT | |
| | TTCTGAAACTGGTGAAAAGCTATCATTGGATGGGCCT | |
| | GGTGCAGATTCCGGCGAACGGCAGCTGGCAGTGGGAA | |
| | GATGGCAGCAGCCTGAGCTATAACCAGCTGACCCTGG | |
| | TGGAAATTCCGAAAGGCAGCTGCGCGGTGTATGGCAG | |
| | CAGCTTTAAAGCGTATACCGAAGATTGCGCGAACCTG | |
| | AACACCTATATTTGCATGAAACGCGCGGTG | |
| CD28 | YMNM | 96 |
| YMNM | | |
| CD28 | PYAP | 97 |
| PYAP | | |
| CD28 | FMNM | 98 |
| FMNM | | |
| CD28 | AYAA | 99 |
| AYAA | | |
| Signal | ATMGWSCIILFLVATATGVHS | 100 |
| peptide | | |
| Signal | ATGGGATGGAGCTGTATCATCCTCTTCTTGGTAGCAA | 101 |
| peptide | CAGCTACCGGTGTGCACTCC | |
| DNA | | |
| sequence | | |
| Anti- | QVQLVQSGAEVKKPGSSVKVSCKASGYAFSYSWINWV | 102 |
| CD20 | RQAPGQGLEWMGRIFPGDGDTDYNGKFKGRVTITADK | |
| (GA101) | STSTAYMELSSLRSEDTAVYYCARNVFDGYWLVYWGQ | |
| heavy | GTLVTVSSASTKGPSVFPLAPSSKSTSGGTAALGCLV | |
| chain | KDYFPEPVTVSWNSGALTSGVHTFPAVLQSSGLYSLS | |
| | SVVTVPSSSLGTQTYICNVNHKPSNTKVDKKVEPKSC | |
| | DKTHTCPPCPAPELLGGPSVFLFPPKPKDTLMISRTP | |
| | EVTCVVVDVSHEDPEVKFNWYVDGVEVHNAKTKPREE | |
| | QYNSTYRVVSVLTVLHQDWLNGKEYKCKVSNKALPAP | |
| | IEKTISKAKGQPREPQVYTLPPSRDELTKNQVSLTCL | |
| | VKGFYPSDIAVEWESNGQPENNYKTTPPVLDSDGSFF | |
| | LYSKLTVDKSRWQQGNVFSCSVMHEALHNHYTQKSLS | |
| | LSP | |
| Anti- | DIVMTQTPLSLPVTPGEPASISCRSSKSLLHSNGITY | 103 |
| CD20 | LYWYLQKPGQSPQLLIYQMSNLVSGVPDRFSGSGSGT | |
| (GA101) | DFTLKISRVEAEDVGVYYCAQNLELPYTFGGGTKVEI | |
| light | KRTVAAPSVFIFPPSDEQLKSGTASVVCLLNNFYPRE | |
| chain | AKVQWKVDNALQSGNSQESVTEQDSKDSTYSLSSTLT | |
| | LSKADYEKHKVYACEVTHQGLSSPVTKSFNRGEC | |
| Anti- | EVQLLESGGGLVQPGGSLRLSCAASGFTFSSYAMSWV | 104 |
| FAP(4B9) | RQAPGKGLEWVSAIIGSGASTYYADSVKGRFTISRDN | |
| PGLALA | SKNTLYLQMNSLRAEDTAVYYCAKGWFGGFNYWGQGT | |
| heavy | LVTVSSASTKGPSVFPLAPSSKSTSGGTAALGCLVKD | |
| chain | YFPEPVTVSWNSGALTSGVHTFPAVLQSSGLYSLSSV | |
| | VTVPSSSLGTQTYICNVNHKPSNTKVDKKVEPKSCDK | |
| | THTCPPCPAPEAAGGPSVFLFPPKPKDTLMISRTPEV | |
| | TCVVVDVSHEDPEVKFNWYVDGVEVHNAKTKPREEQY | |
| | NSTYRVVSVLTVLHQDWLNGKEYKCKVSNKALGAPIE | |
| | KTISKAKGQPREPQVYTLPPSRDELTKNQVSLTCLVK | |
| | GFYPSDIAVEWESNGQPENNYKTTPPVLDSDGSFFLY | |
| | SKLTVDKSRWQQGNVFSCSVMHEALHNHYTQKSLSLS | |
| | P | |
| Anti- | EIVLTQSPGTLSLSPGERATLSCRASQSVTSSYLAWY | 105 |
| FAP(4B9) | QQKPGQAPRLLINVGSRRATGIPDRFSGSGSGTDFTL | |
| light | TISRLEPEDFAVYYCQQGIMLPPTFGQGTKVEIKRTV | |
| chain | AAPSVFIFPPSDEQLKSGTASVVCLLNNFYPREAKVQ | |
| | WKVDNALQSGNSQESVTEQDSKDSTYSLSSTLTLSKA | |
| | DYEKHKVYACEVTHQGLSSPVTKSFNRGEC | |
| Anti- | EVQLVESGGGLVQPGRSLRLSCAASGFTVSSYWMHWV | 106 |
| CEA | RQAPGKGLEWVGFIRNKANGGTTEYAASVKGRFTISR | |
| (A5B7) | DDSKNTLYLQMNSLRAEDTAVYYCARDRGLRFYFDYW | |
| PGLALA | GQGTTVTVSSASTKGPSVFPLAPSSKSTSGGTAALGC | |
| heavy | LVKDYFPEPVTVSWNSGALTSGVHTFPAVLQSSGLYS | |
| chain | LSSVVTVPSSSLGTQTYICNVNHKPSNTKVDKKVEPK | |
| | SCDKTHTCPPCPAPEAAGGPSVFLFPPKPKDTLMISR | |
| | TPEVTCVVVDVSHEDPEVKFNWYVDGVEVHNAKTKPR | |
| | EEQYNSTYRVVSVLTVLHQDWLNGKEYKCKVSNKALG | |
| | APIEKTISKAKGQPREPQVYTLPPSRDELTKNQVSLT | |
| | CLVKGFYPSDIAVEWESNGQPENNYKTTPPVLDSDGS | |
| | FFLYSKLTVDKSRWQQGNVFSCSVMHEALHNHYTQKS | |
| | LSLSP | |
| Anti- | QAVLTQPASLSASPGASASLTCTLRRGINVGAYSIYW | 107 |
| CEA | YQQKPGSPPQYLLRYKSDSDKQQGSGVSSRFSASKDA | |
| (A5B7) | SANAGILLISGLQSEDEADYYCMIWHSGASAVFGGGT | |
| light | KLTVLRTVAAPSVFIFPPSDEQLKSGTASVVCLLNNF | |
| chain | YPREAKVQWKVDNALQSGNSQESVTEQDSKDSTYSLS | |
| | STLTLSKADYEKHKVYACEVTHQGLSSPVTKSFNRGE | |
| | C | |
| Anti- | QVQLVQSGAEVKKPGSSVKVSCKASGFNIKDTYMHWV | 108 |
| CEA | RQAPGQGLEWMGRIDPANGNSKYVPKFQGRVTITADT | |
| (T84. | STSTAYMELSSLRSEDTAVYYCAPFGYYVSDYAMAYW | |
| 66LCHA) | GQGTLVTVSSASTKGPSVFPLAPSSKSTSGGTAALGC | |
| PGLALA | LVKDYFPEPVTVSWNSGALTSGVHTFPAVLQSSGLYS | |
| heavy | LSSVVTVPSSSLGTQTYICNVNHKPSNTKVDKKVEPK | |
| chain | SCDKTHTCPPCPAPEAAGGPSVFLFPPKPKDTLMISR | |
| | TPEVTCVVVDVSHEDPEVKFNWYVDGVEVHNAKTKPR | |
| | EEQYNSTYRVVSVLTVLHQDWLNGKEYKCKVSNKALG | |
| | APIEKTISKAKGQPREPQVYTLPPSRDELTKNQVSLT | |
| | CLVKGFYPSDIAVEWESNGQPENNYKTTPPVLDSDGS | |
| | FFLYSKLTVDKSRWQQGNVFSCSVMHEALHNHYTQKS | |
| | LSLSP | |
| Anti- | EIVLTQSPATLSLSPGERATLSCRAGESVDIFGVGFL | 109 |
| CEA | HWYQQKPGQAPRLLIYRASNRATGIPARFSGSGSGTD | |
| (T84. | FTLTISSLEPEDFAVYYCQQTNEDPYTFGQGTKLEIK | |
| 66LCHA) | RTVAAPSVFIFPPSDEQLKSGTASVVCLLNNFYPREA | |
| light | KVQWKVDNALQSGNSQESVTEQDSKDSTYSLSSTLTL | |
| chain | SKADYEKHKVYACEVTHQGLSSPVTKSFNRGEC | |
| Anti- | QVQLVQSGAEVKKPGASVKVSCKASGYTFTEFGMNWV | 110 |
| CEA | RQAPGQGLEWMGWINTKTGEATYVEEFKGRVTFTTDT | |
| (CH1A- | STSTAYMELRSLRSDDTAVYYCARWDFAYYVEAMDYW | |
| 1A98/ | GQGTTVTVSSASTKGPSVFPLAPSSKSTSGGTAALGC | |
| 992F1) | LVKDYFPEPVTVSWNSGALTSGVHTFPAVLQSSGLYS | |
| PGLALA | LSSVVTVPSSSLGTQTYICNVNHKPSNTKVDKKVEPK | |
| heavy | SCDKTHTCPPCPAPEAAGGPSVFLFPPKPKDTLMISR | |
| chain | TPEVTCVVVDVSHEDPEVKFNWYVDGVEVHNAKTKPR | |
| | EEQYNSTYRVVSVLTVLHQDWLNGKEYKCKVSNKALG | |
| | APIEKTISKAKGQPREPQVCTLPPSRDELTKNQVSLS | |
| | CAVKGFYPSDIAVEWESNGQPENNYKTTPPVLDSDGS | |
| | FFLVSKLTVDKSRWQQGNVFSCSVMHEALHNHYTQKS | |
| | LSLSP | |
| Anti- | DIQMTQSPSSLSASVGDRVTITCKASAAVGTYVAWYQ | 111 |
| CEA | QKPGKAPKLLIYSASYRKRGVPSRFSGSGSGTDFTLT | |
| (CH1A- | ISSLQPEDFATYYCHQYYTYPLFTFGQGTKLEIKRTV | |
| 1A98/ | AAPSVFIFPPSDEQLKSGTASVVCLLNNFYPREAKVQ | |
| 992F1) | WKVDNALQSGNSQESVTEQDSKDSTYSLSSTLTLSKA | |
| light | DYEKHKVYACEVTHQGLSSPVTKSFNRGEC | |
| chain | | |
| Anti- | EVQLVESGGGVVQPGRSLRLSCSASGFDFTTYWMSWV | 112 |
| CEA | RQAPGKGLEWIGEIHPDSSTINYAPSLKDRFTISRDN | |
| (hMN14) | AKNTLFLQMDSLRPEDTGVYFCASLYFGFPWFAYWGQ | |
| PGLALA | GTPVTVSSASTKGPSVFPLAPSSKSTSGGTAALGCLV | |
| heavy | KDYFPEPVTVSWNSGALTSGVHTFPAVLQSSGLYSLS | |
| chain | SVVTVPSSSLGTQTYICNVNHKPSNTKVDKKVEPKSC | |
| | DKTHTCPPCPAPEAAGGPSVFLFPPKPKDTLMISRTP | |
| | EVTCVVVDVSHEDPEVKFNWYVDGVEVHNAKTKPREE | |
| | QYNSTYRVVSVLTVLHQDWLNGKEYKCKVSNKALGAP | |
| | IEKTISKAKGQPREPQVYTLPPSRDELTKNQVSLTCL | |
| | VKGFYPSDIAVEWESNGQPENNYKTTPPVLDSDGSFF | |
| | LYSKLTVDKSRWQQGNVFSCSVMHEALHNHYTQKSLS | |
| | LSP | |
| Anti- | DIQLTQSPSSLSASVGDRVTITCKASQDVGTSVAWYQ | 113 |
| CEA | QKPGKAPKLLIYWTSTRHTGVPSRFSGSGSGTDFTFT | |
| (hMN14) | ISSLQPEDIATYYCQQYSLYRSFGQGTKVEIKRTVAA | |
| light | PSVFIFPPSDEQLKSGTASVVCLLNNFYPREAKVQWK | |
| chain | VDNALQSGNSQESVTEQDSKDSTYSLSSTLTLSKADY | |
| | EKHKVYACEVTHQGLSSPVTKSFNRGEC | |
| Anti- | QVQLVQSGAEVKKPGSSVKVSCKASGGTFSSYAISWV | 114 |
| TNC | RQAPGQGLEWMGGIIPIFGTANYAQKFQGRVTITADK | |
| (2B10) | STSTAYMELSSLRSEDTAVYYCARLYGYAYYGAFDYW | |
| PGLALA | GQGTTVTVSSASTKGPSVFPLAPSSKSTSGGTAALGC | |
| heavy | LVKDYFPEPVTVSWNSGALTSGVHTFPAVLQSSGLYS | |
| chain | LSSVVTVPSSSLGTQTYICNVNHKPSNTKVDKKVEPK | |
| | SCDKTHTCPPCPAPEAAGGPSVFLFPPKPKDTLMISR | |
| | TPEVTCVVVDVSHEDPEVKFNWYVDGVEVHNAKTKPR | |
| | EEQYNSTYRVVSVLTVLHQDWLNGKEYKCKVSNKALG | |
| | APIEKTISKAKGQPREPQVCTLPPSRDELTKNQVSLS | |
| | CAVKGFYPSDIAVEWESNGQPENNYKTTPPVLDSDGS | |
| | FFLVSKLTVDKSRWQQGNVFSCSVMHEALHNHYTQKS | |
| | LSLSP | |
| Anti- | DIQMTQSPSSLSASVGDRVTITCRASQGIRNDLGWYQ | 115 |
| TNC | QKPGKAPKRLIYAASSLQSGVPSRFSGSGSGTEFTLT | |
| (2B10) | ISSLQPEDFATYYCLQNGLQPATFGQGTKVEIKRTVA | |
| light | APSVFIFPPSDEQLKSGTASVVCLLNNFYPREAKVQW | |
| chain | KVDNALQSGNSQESVTEQDSKDSTYSLSSTLTLSKAD | |
| | YEKHKVYACEVTHQGLSSPVTKSFNRGEC | |
| (G4S)n | (G4S)n | 116 |
| (SG4)n | (SG4)n | 117 |
| (G4S)n | (G4S)n | 118 |
| G4(SG4)n | G4(SG4)n | 119 |
| Human | ASTKGPSVFPLAPSSKSTSGGTAALGCLVKDYFPEPV | 120 |
| IgG1 Fc | TVSWNSGALTSGVHTFPAVLQSSGLYSLSSVVTVPSS | |
| | SLGTQTYICNVNHKPSNTKVDKKVEPKSCDKTHTCPP | |
| | CPAPELLGGPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKA | |
| | KGQPREPQVYTLPPSRDELTKNQVSLTCLVKGFYPSD | |
| | IAVEWESNGQPENNYKTTPPVLDSDGSFFLYSKLTVD | |
| | KSRWQQGNVFSCSVMHEALHNHYTQKSLSLSP | |
| Human | MRNQAPGRPKGATFPPRRPTGSRAPPLAPELRAKQRP | 121 |
| CD8 | GERVMALPVTALLLPLALLLHAARPSQFRVSPLDRTW | |
| | NLGETVELKCQVLLSNPTSGCSWLFQPRGAAASPTFL | |
| | LYLSQNKPKAAEGLDTQRFSGKRLGDTFVLTLSDFRR | |
| | ENEGYYFCSALSNSIMYFSHFVPVFLPAKPTTTPAPR | |
| | PPTPAPTIASQPLSLRPEACRPAAGGAVHTRGLDFAC | |
| | DIYIWAPLAGTCGVLLLSLVITLYCNHRNRRRVCKCP | |
| | RPVVKSGDKPSLSARYV | |
| Human | ATGCGCAACCAGGCGCCGGGCCGCCCGAAAGGCGCGA | 122 |
| CD8 | CCTTTCCGCCGCGCCGCCCGACCGGCAGCCGCGCGCC | |
| | GCCGCTGGCGCCGGAACTGCGCGCGAAACAGCGCCCG | |
| | GGCGAACGCGTGATGGCGCTGCCGGTGACCGCGCTGC | |
| | TGCTGCCGCTGGCGCTGCTGCTGCATGCGGCGCGCCC | |
| | GAGCCAGTTTCGCGTGAGCCCGCTGGATCGCACCTGG | |
| | AACCTGGGCGAAACCGTGGAACTGAAATGCCAGGTGC | |
| | TGCTGAGCAACCCGACCAGCGGCTGCAGCTGGCTGTT | |
| | TCAGCCGCGCGGCGCGGCGGCGAGCCCGACCTTTCTG | |
| | CTGTATCTGAGCCAGAACAAACCGAAAGCGGCGGAAG | |
| | GCCTGGATACCCAGCGCTTTAGCGGCAAACGCCTGGG | |
| | CGATACCTTTGTGCTGACCCTGAGCGATTTTCGCCGC | |
| | GAAAACGAAGGCTATTATTTTTGCAGCGCGCTGAGCA | |
| | ACAGCATTATGTATTTTAGCCATTTTGTGCCGGTGTT | |
| | TCTGCCGGCGAAACCGACCACCACCCCGGCGCCGCGC | |
| | CCGCCGACCCCGGCGCCGACCATTGCGAGCCAGCCGC | |
| | TGAGCCTGCGCCCGGAAGCGTGCCGCCCGGCGGCGGG | |
| | CGGCGCGGTGCATACCCGCGGCCTGGATTTTGCGTGC | |
| | GATATTTATATTTGGGCGCCGCTGGCGGGCACCTGCG | |
| | GCGTGCTGCTGCTGAGCCTGGTGATTACCCTGTATTG | |
| | CAACCATCGCAACCGCCGCCGCGTGTGCAAATGCCCG | |
| | CGCCCGGTGGTGAAAAGCGGCGATAAACCGAGCCTGA | |
| | GCGCGCGCTATGTG | |
| Murine | MASPLTRFLSLNLLLMGESIILGSGEAKPQAPELRIF | 123 |
| CD8 | PKKMDAELGQKVDLVCEVLGSVSQGCSWLFQNSSSKL | |
| | PQPTFVVYMASSHNKITWDEKLNSSKLFSAVRDTNNK | |
| | YVLTLNKFSKENEGYYFCSVISNSVMYFSSVVPVLQK | |
| | VNSTTTKPVLRTPSPVHPTGTSQPQRPEDCRPRGSVK | |
| | GTGLDFACDIYIWAPLAGICVAPLLSLIITLICYHRS | |
| | RKRVCKCPRPLVRQEGKPRPSEKIV | |
| Murine | ATGGCGAGCCCGCTGACCCGCTTTCTGAGCCTGAACC | 124 |
| CD8 | TGCTGCTGATGGGCGAAAGCATTATTCTGGGCAGCGG | |
| | CGAAGCGAAACCGCAGGCGCCGGAACTGCGCATTTTT | |
| | CCGAAAAAAATGGATGCGGAACTGGGCCAGAAAGTGG | |
| | ATCTGGTGTGCGAAGTGCTGGGCAGCGTGAGCCAGGG | |
| | CTGCAGCTGGCTGTTTCAGAACAGCAGCAGCAAACTG | |
| | CCGCAGCCGACCTTTGTGGTGTATATGGCGAGCAGCC | |
| | ATAACAAAATTACCTGGGATGAAAAACTGAACAGCAG | |
| | CAAACTGTTTAGCGCGGTGCGCGATACCAACAACAAA | |
| | TATGTGCTGACCCTGAACAAATTTAGCAAAGAAAACG | |
| | AAGGCTATTATTTTTGCAGCGTGATTAGCAACAGCGT | |
| | GATGTATTTTAGCAGCGTGGTGCCGGTGCTGCAGAAA | |
| | GTGAACAGCACCACCACCAAACCGGTGCTGCGCACCC | |
| | CGAGCCCGGTGCATCCGACCGGCACCAGCCAGCCGCA | |
| | GCGCCCGGAAGATTGCCGCCCGCGCGGCAGCGTGAAA | |
| | GGCACCGGCCTGGATTTTGCGTGCGATATTTATATTT | |
| | GGGCGCCGCTGGCGGGCATTTGCGTGGCGCCGCTGCT | |
| | GAGCCTGATTATTACCCTGATTTGCTATCATCGCAGC | |
| | CGCAAACGCGTGTGCAAATGCCCGCGCCCGCTGGTGC | |
| | GCCAGGAAGGCAAACCGCGCCCGAGCGAAAAAATTGT | |
| | G | |

TABLE 9
Exemplary VH1VL1 P329G-CAR amino acid
sequences: CDR definition according to Kabat

| | | SEQ |
|---|---|---|
| | | ID |
| Construct | Amino acid sequence | NO |
| VH1 CDR H1 | see Table 2 | 1 |
| VH1 CDR H2 | see Table 6 | 40 |
| VH1 CDR H3 | see Table 2 | 3 |
| VL1 CDR L1 | see Table 2 | 4 |
| VL1 CDR L2 | see Table 2 | 5 |
| VL1 CDR L3 | see Table 2 | 6 |
| VH1VL1- | EVQLVESGGGLVQPGGSLRLSCAASGFDFSRYW | 125 |
| CD8ATD- | MNWVRQAPGKGLEWVGEITPDSSTINYTPSLKG | |
| CD137CSD- | RFTISRDNAKNSLYLQMNSLRAEDTAVYYCVRP | |
| CD3zSSD | YDYGAWFASWGQGTLVTVSSGGGGSGGGGSGGG | |
| fusion | GSGGGGSQAVVTQEPSLTVSPGGTVTLTCRSST | |
| | GAVTTSNYANWVQEKPDHLFTGLIGGTNKRAPG | |
| | TPARFSGSLLGGKAALTLSGAQPEDEAEYYCAL | |
| | WYSNHWVFGGGTKLTVLGGGGSLKPTTTPAPRP | |
| | PTPAPTIASQPLSLRPEACRPAAGGAVHTRGLD | |
| | FACDIYIWAPLAGTCGVLLLSLVITKRGRKKLL | |
| | YIFKQPFMRPVQTTQEEDGCSCRFPEEEEGGCE | |
| | LRVKFSRSADAPAYQQGQNQLYNELNLGRREEY | |
| | DVLDKRRGRDPEMGGKPRRKNPQEGLYNELQKD | |
| | KMAEAYSEIGMKGERRRGKGHDGLYQGLSTATK | |
| | DTYDALHMQALPPR | |
| VH1 VH | see Table 6 | 41 |
| VL1 VL | see Table 2 | 9 |
| VH1VL1 | EVQLVESGGGLVQPGGSLRLSCAASGFDFSRYW | 126 |
| scFv | MNWVRQAPGKGLEWVGEITPDSSTINYTPSLKG | |
| | RFTISRDNAKNSLYLQMNSLRAEDTAVYYCVRP | |
| | YDYGAWFASWGQGTLVTVSSGGGGSGGGGSGGG | |
| | GSGGGGSQAVVTQEPSLTVSPGGTVTLTCRSST | |
| | GAVTTSNYANWVQEKPDHLFTGLIGGTNKRAPG | |
| | TPARFSGSLLGGKAALTLSGAQPEDEAEYYCAL | |
| | WYSNHWVFGGGTKLTVL | |
| CD8ATD | see Table 2 | 11 |
| CD137CSD | see Table 2 | 12 |
| CD3zSSD | see Table 2 | 13 |
| CD28ATD- | see Table 2 | 14 |
| CD137CSD- | | |
| CD3zSSD | | |
| eGFP | see Table 2 | 15 |
| (G4S)4 | see Table 2 | 16 |
| linker | | |
| G4S linker | see Table 2 | 17 |
| T2A linker | see Table 2 | 18 |
| CD8stalk | see Table 2 | 19 |

TABLE 10
Exemplary VH2VL1 P329G-CAR amino acid
sequences: CDR definition according to Kabat

| | | SEQ |
|---|---|---|
| | | ID |
| Construct | Amino acid sequence | NO |
| VH2 CDR H1 | see Table 2 | 1 |
| VH2 CDR H2 | see Table 2 | 2 |
| VH2 CDR H3 | see Table 2 | 3 |
| VL1 CDR L1 | see Table 2 | 4 |
| VL1 CDR L2 | see Table 2 | 5 |
| VL1 CDR L3 | see Table 2 | 6 |
| VH2VL1- | EVQLVESGGGLVQPGGSLRLSCAASGFDFSRYW | 127 |
| CD8ATD- | MNWVRQAPGKGLEWVGEITPDSSTINYAPSLKG | |
| CD137CSD- | RFTISRDNAKNSLYLQMNSLRAEDTAVYYCVRP | |
| CD3zSSD | YDYGAWFASWGQGTLVTVSSGGGGSGGGGSGGG | |
| fusion | GSGGGGSQAVVTQEPSLTVSPGGTVTLTCRSST | |
| | GAVTTSNYANWVQEKPDHLFTGLIGGTNKRAPG | |
| | TPARFSGSLLGGKAALTLSGAQPEDEAEYYCAL | |
| | WYSNHWVFGGGTKLTVLGGGGSLKPTTTPAPRP | |
| | PTPAPTIASQPLSLRPEACRPAAGGAVHTRGLD | |
| | FACDIYIWAPLAGTCGVLLLSLVITKRGRKKLL | |
| | YIFKQPFMRPVQTTQEEDGCSCRFPEEEEGGCE | |
| | LRVKFSRSADAPAYQQGQNQLYNELNLGRREEY | |
| | DVLDKRRGRDPEMGGKPRRKNPQEGLYNELQKD | |
| | KMAEAYSEIGMKGERRRGKGHDGLYQGLSTATK | |
| | DTYDALHMQALPPR | |
| VH2 VH | see Table 6 | 44 |
| VL1 VL | see Table 2 | 9 |
| VH2VL1 | EVQLVESGGGLVQPGGSLRLSCAASGFDFSRYW | 128 |
| scFv | MNWVRQAPGKGLEWVGEITPDSSTINYAPSLKG | |
| | RFTISRDNAKNSLYLQMNSLRAEDTAVYYCVRP | |
| | YDYGAWFASWGQGTLVTVSSGGGGSGGGGSGGG | |
| | GSGGGGSQAVVTQEPSLTVSPGGTVTLTCRSST | |
| | GAVTTSNYANWVQEKPDHLFTGLIGGTNKRAPG | |
| | TPARFSGSLLGGKAALTLSGAQPEDEAEYYCAL | |
| | WYSNHWVFGGGTKLTVL | |
| CD8ATD | see Table 2 | 11 |
| CD137CSD | see Table 2 | 12 |
| CD3zSSD | see Table 2 | 13 |
| CD28ATD- | see Table 2 | 14 |
| CD137CSD- | | |
| CD3zSSD | | |
| eGFP | see Table 2 | 15 |
| (G4S)4 | see Table 2 | 16 |
| linker | | |
| G4S linker | see Table 2 | 17 |
| T2A linker | see Table 2 | 18 |
| CD8stalk | see Table 2 | 19 |

TABLE 11
Exemplary heterodimeric antibody sequences:
CDR definition according to Kabat

| | | SEQ ID |
|---|---|---|
| Construct | Amino acid sequence | NO |
| Anti-CD20 | QVQLVQSGAEVKKPGSSVKVSCKASGYAFSYSWINWVR | 129 |
| (GA101) | QAPGQGLEWMGRIFPGDGDTDYNGKFKGRVTITADKST | |
| heterodimeric | STAYMELSSLRSEDTAVYYCARNVFDGYWLVYWGQGTL | |
| IgG1 HC | VTVSSASTKGPSVFPLAPSSKSTSGGTAALGCLVKDYF | |
| (knob, P329G) | PEPVTVSWNSGALTSGVHTFPAVLQSSGLYSLSSVVTV | |
| defucosylated | PSSSLGTQTYICNVNHKPSNTKVDKKVEPKSCDKTHTC | |
| | PPCPAPELLGGPSVFLFPPKPKDTLMISRTPEVTCVVV | |
| | DVSHEDPEVKFNWYVDGVEVHNAKTKPREEQYNSTYRV | |
| | VSVLTVLHQDWLNGKEYKCKVSNKALGAPIEKTISKAK | |
| | GQPREPQVYTLPPCRDELTKNQVSLWCLVKGFYPSDIA | |
| | VEWESNGQPENNYKTTPPVLDSDGSFFLYSKLTVDKSR | |
| | WQQGNVFSCSVMHEALHNHYTQKSLSLSP | |
| Anti-CD20 | QVQLVQSGAEVKKPGSSVKVSCKASGYAFSYSWINWVR | 130 |
| (GA101) | QAPGQGLEWMGRIFPGDGDTDYNGKFKGRVTITADKST | |
| heterodimeric | STAYMELSSLRSEDTAVYYCARNVFDGYWLVYWGQGTL | |
| IgG1 HC | VTVSSASTKGPSVFPLAPSSKSTSGGTAALGCLVKDYF | |
| (hole), | PEPVTVSWNSGALTSGVHTFPAVLQSSGLYSLSSVVTV | |
| defucosylated | PSSSLGTQTYICNVNHKPSNTKVDKKVEPKSCDKTHTC | |
| | PPCPAPELLGGPSVFLFPPKPKDTLMISRTPEVTCVVV | |
| | DVSHEDPEVKFNWYVDGVEVHNAKTKPREEQYNSTYRV | |
| | VSVLTVLHQDWLNGKEYKCKVSNKALPAPIEKTISKAK | |
| | GQPREPQVCTLPPSRDEL | |
| Anti-CD20 | DIVMTQTPLSLPVTPGEPASISCRSSKSLLHSNGITYL | 131 |
| (GA101) IgG1 | YWYLQKPGQSPQLLIYQMSNLVSGVPDRFSGSGSGTDF | |
| LC | TLKISRVEAEDVGVYYCAQNLELPYTFGGGTKVEIKRT | |
| | VAAPSVFIFPPSDEQLKSGTASVVCLLNNFYPREAKVQ | |
| | WKVDNALQSGNSQESVTEQDSKDSTYSLSSTLTLSKAD | |
| | YEKHKVYACEVTHQGLSSPVTKSFNRGEC | |
| Anti-CD20 | QVQLVQSGAEVKKPGSSVKVSCKASGYAFSYSWINWVR | 132 |
| (GA101) IgG1 | QAPGQGLEWMGRIFPGDGDTDYNGKFKGRVTITADKST | |
| HC | STAYMELSSLRSEDTAVYYCARNVFDGYWLVYWGQGTL | |
| defucosylated | VTVSSASTKGPSVFPLAPSSKSTSGGTAALGCLVKDYF | |
| | PEPVTVSWNSGALTSGVHTFPAVLQSSGLYSLSSVVTV | |
| | PSSSLGTQTYICNVNHKPSNTKVDKKVEPKSCDKTHTC | |
| | PPCPAPELLGGPSVFLFPPKPKDTLMISRTPEVTCVVV | |
| | DVSHEDPEVKFNWYVDGVEVHNAKTKPREEQYNSTYRV | |
| | VSVLTVLHQDWLNGKEYKCKVSNKALPAPIEKTISKAK | |
| | GQPREPQVYTLPPSRDELTKNQVSLTCLVKGFYPSDIA | |
| | VEWESNGQPENNYKTTPPVLDSDGSFFLYSKLTVDKSR | |
| | WQQGNVFSCSVMHEALHNHYTQKSLSLSP | |
| Anti-CD20 | DIVMTQTPLSLPVTPGEPASISCRSSKSLLHSNGITYL | 131 |
| (GA101) IgG1 | YWYLQKPGQSPQLLIYQMSNLVSGVPDRFSGSGSGTDF | |
| LC | TLKISRVEAEDVGVYYCAQNLELPYTFGGGTKVEIKRT | |
| | VAAPSVFIFPPSDEQLKSGTASVVCLLNNFYPREAKVQ | |
| | WKVDNALQSGNSQESVTEQDSKDSTYSLSSTLTLSKAD | |
| | YEKHKVYACEVTHQGLSSPVTKSFNRGEC | |
| Anti-CD20 | QVQLVQSGAEVKKPGSSVKVSCKASGYAFSYSWINWVR | 132 |
| (GA101) IgG1 | QAPGQGLEWMGRIFPGDGDTDYNGKFKGRVTITADKST | |
| HC | STAYMELSSLRSEDTAVYYCARNVFDGYWLVYWGQGTL | |
| | VTVSSASTKGPSVFPLAPSSKSTSGGTAALGCLVKDYF | |
| | PEPVTVSWNSGALTSGVHTFPAVLQSSGLYSLSSVVTV | |
| | PSSSLGTQTYICNVNHKPSNTKVDKKVEPKSCDKTHTC | |
| | PPCPAPELLGGPSVFLFPPKPKDTLMISRTPEVTCVVV | |
| | DVSHEDPEVKFNWYVDGVEVHNAKTKPREEQYNSTYRV | |
| | VSVLTVLHQDWLNGKEYKCKVSNKALPAPIEKTISKAK | |
| | GQPREPQVYTLPPSRDELTKNQVSLTCLVKGFYPSDIA | |
| | VEWESNGQPENNYKTTPPVLDSDGSFFLYSKLTVDKSR | |
| | WQQGNVFSCSVMHEALHNHYTQKSLSLSP | |
| Anti-CD20 | DIVMTQTPLSLPVTPGEPASISCRSSKSLLHSNGITYL | 131 |
| (GA101) IgG1 | YWYLQKPGQSPQLLIYQMSNLVSGVPDRFSGSGSGTDF | |
| LC | TLKISRVEAEDVGVYYCAQNLELPYTFGGGTKVEIKRT | |
| | VAAPSVFIFPPSDEQLKSGTASVVCLLNNFYPREAKVQ | |
| | WKVDNALQSGNSQESVTEQDSKDSTYSLSSTLTLSKAD | |
| | YEKHKVYACEVTHQGLSSPVTKSFNRGEC | |
| Anti-CD20 | QVQLVQSGAEVKKPGSSVKVSCKASGYAFSYSWINWVR | 133 |
| (GA101) | QAPGQGLEWMGRIFPGDGDTDYNGKFKGRVTITADKST | |
| P329G LALA | STAYMELSSLRSEDTAVYYCARNVFDGYWLVYWGQGTL | |
| IgG1 HC | VTVSSASTKGPSVFPLAPSSKSTSGGTAALGCLVKDYF | |
| | PEPVTVSWNSGALTSGVHTFPAVLQSSGLYSLSSVVTV | |
| | PSSSLGTQTYICNVNHKPSNTKVDKKVEPKSCDKTHTC | |
| | PPCPAPEAAGGPSVFLFPPKPKDTLMISRTPEVTCVVV | |
| | DVSHEDPEVKFNWYVDGVEVHNAKTKPREEQYNSTYRV | |
| | VSVLTVLHQDWLNGKEYKCKVSNKALGAPIEKTISKAK | |
| | GQPREPQVYTLPPSRDELTKNQVSLTCLVKGFYPSDIA | |
| | VEWESNGQPENNYKTTPPVLDSDGSFFLYSKLTVDKSR | |
| | WQQGNVFSCSVMHEALHNHYTQKSLSLSP | |
| Anti-CD20 | DIVMTQTPLSLPVTPGEPASISCRSSKSLLHSNGITYL | 131 |
| (GA101) IgG1 | YWYLQKPGQSPQLLIYQMSNLVSGVPDRFSGSGSGTDF | |
| LC | TLKISRVEAEDVGVYYCAQNLELPYTFGGGTKVEIKRT | |
| | VAAPSVFIFPPSDEQLKSGTASVVCLLNNFYPREAKVQ | |
| | WKVDNALQSGNSQESVTEQDSKDSTYSLSSTLTLSKAD | |
| | YEKHKVYACEVTHQGLSSPVTKSFNRGEC | |

### EXAMPLES

The following are examples of methods and compositions of the invention. It is understood that various other embodiments may be practiced, given the general description provided above.

### Recombinant DNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturers' instructions. General information regarding the nucleotide sequences of human immunoglobulins light and heavy chains is given in: Kabat, E. A. et al., (1991) Sequences of Proteins of Immunological Interest, 5^{th} ed., NIH Publication No. 91-3242.

### DNA Sequencing

DNA sequences were determined by double strand sequencing.

### Gene Synthesis

Desired gene segments where required were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### Production of IgG-Like Proteins in HEK293 EBNA or CHO EBNA Cells

Antibodies and bispecific antibodies were generated by transient transfection of HEK293 EBNA cells or CHO EBNA cells. Cells were centrifuged and, medium was replaced by pre-warmed CD CHO medium (Thermo Fisher, Cat No 10743029). Expression vectors were mixed in CD CHO medium, PEI (Polyethylenimine, Polysciences, Inc, Cat No 23966-1) was added, the solution vortexed and incubated for 10 minutes at room temperature. Afterwards, cells (2 Mio/ml) were mixed with the vector/PEI solution, transferred to a flask and incubated for 3 hours at 37° C. in a shaking incubator with a 5% C02 atmosphere. After the incubation, Excell medium with supplements (80% of total volume) was added (W. Zhou and A. Kantardjieff, Mammalian Cell Cultures for Biologics Manufacturing, DOI: 10.1007/978-3-642-54050-9; 2014). One day after transfection, supplements (Feed, 12% of total volume) were added. Cell supernatants were harvested after 7 days by centrifugation and subsequent filtration (0.2 μm filter), and proteins were purified from the harvested supernatant by standard methods as indicated below.

### Production of IgG-Like Proteins in CHO K1 Cells

Alternatively, the antibodies and bispecific antibodies described herein were prepared by Evitria using their proprietary vector system with conventional (non-PCR based) cloning techniques and using suspension-adapted CHO K1 cells (originally received from ATCC and adapted to serum-free growth in suspension culture at Evitria). For the production, Evitria used its proprietary, animal-component free and serum-free media (eviGrow and eviMake2) and its proprietary transfection reagent (eviFect). Supernatant was harvested by centrifugation and subsequent filtration (0.2 μm filter) and, proteins were purified from the harvested supernatant by standard methods.

### Purification of IgG-Like Proteins

Proteins were purified from filtered cell culture supernatants referring to standard protocols. In brief, Fc containing proteins were purified from cell culture supernatants by Protein A-affinity chromatography (equilibration buffer: 20 mM sodium citrate, 20 mM sodium phosphate, pH 7.5; elution buffer: 20 mM sodium citrate, pH 3.0). Elution was achieved at pH 3.0 followed by immediate pH neutralization of the sample. The protein was concentrated by centrifugation (Millipore Amicon® ULTRA-15 (Art.Nr.: UFC903096), and aggregated protein was separated from monomeric protein by size exclusion chromatography in 20 mM histidine, 140 mM sodium chloride, pH 6.0.

### Analytics of IgG-Like Proteins

The concentrations of purified proteins were determined by measuring the absorption at 280 nm using the mass extinction coefficient calculated on the basis of the amino acid sequence according to Pace, et al., Protein Science, 1995, 4, 2411-1423. Purity and molecular weight of the proteins were analyzed by CE-SDS in the presence and absence of a reducing agent using a LabChipGXII or LabChip GX Touch (Perkin Elmer) (Perkin Elmer). Determination of the aggregate content was performed by HPLC chromatography at 25° C. using analytical size-exclusion column (TSKgel G3000 SW XL or UP-SW3000) equilibrated in running buffer (200 mM KH2PO4, 250 mM KCl pH 6.2, 0.02% NaN3).

### Preparation of Lentivirus Supernatants and Transduction of Jurkat-NFAT Cells

Lipofectamine LTX™-based transfection was performed using ˜80% confluent Hek293T cells (ATCC CRL3216) and CAR encoding transfer vectors as well as packaging vectors pCAG-VSVG and psPAX2 at a 2:2:1 molar ratio (Giry-Laterriere M, et al Methods Mol Biol. 2011; 737:183-209, Myburgh R, et al Mol Ther Nucleic Acids. 2014). After 66 h, the supernatant was collected, centrifuged for 5 min at 350×g and filtrated through a 0.45-μm polyethersulfon filter to harvest and purify the virus particles. Virus particles were either used directly or concentrated (Lenti-x-Concentrator, Takara) and used for spinfection of Jurkat NFAT T cells (GloResponse Jurkat NFAT-RE-luc2P, Promega #CS176501 at 900×g for 2 h and 31° C.

### Jurkat NFAT Activation Assay

The Jurkat NFAT activation assay measures T cell activation of a human acute lymphatic leukemia reporter cell line (GloResponse Jurkat NFAT-RE-luc2P, Promega #CS176501). This immortalized T cell line is genetically engineered to stably express a luciferase reporter driven by an NFAT-response element (NFAT-RE). Further, the cell line expresses a chimeric antigen receptor (CAR) construct possessing a CD3z signaling domain. Binding of the CAR to an immobilized adapter molecule (e.g. a tumor antigen bound adapter molecule) leads to CAR crosslinking resulting in T cell activation and in the expression of luciferase. After addition of a substrate the cellular changes of the NFAT activity can be measured as relative light units (Darowski et al. Protein Engineering, Design and Selection, Volume 32, Issue 5, May 2019, Pages 207-218, https://doi.org/10.1093/protein/gzz027). In general, the assay was performed in a 384 plate (Falcon #353963 white, clear bottom). Target cells (CAR-Jurkat-NFAT cells) and effector cells were seeded in a 1:5 ratio (2000 target cells and 10 000 effector cells) in 10 μl each, in RPMI-1640+10% FCS+1% Glutamax (growth medium) in triplicates. Further, a serial dilution of the antibody of interest was prepared in growth medium to obtain a final concentrations ranging from 67 nM to 0.000067 nM in the assay plate with a final volume of 30 μl per well in total. The 384 well plate was centrifuged for 1 min at 300 g and RT and incubated at 37° C. and 5% CO₂ in a humidity atmosphere. After 7 h incubation 20% of the final volume of ONE-Glo™ Luciferase Assay (E6120, Promega) was added, and plates were centrifuged for 1 min at 350×g. Afterwards, the relative luminescence units (RLU) per s/well were measured immediately using a Tecan microplate reader. Concentration-response curves were fitted and EC₅₀ values were calculated using GraphPadPrism version 7. As p value the New England Journal of Medicine style was used as listed in GraphPadPrism 7. Meaning *=P≤0,033; **=P≤0,002; ***=P≤0,001.

### Example 1

### Generation and Characterization of Humanized Anti-P329G Antibodies

Parental and humanized anti-P329G antibodies were produced in HEK cells and purified by ProteinA affinity chromatography and size exclusion chromatography. All antibodies were purified in good quality (Table 2).

TABLE 2
Biochemical analysis of anti-P329G antibodies. Monomer content
determined by analytical size exclusion chromatography. Purity
determined by non-reducing SDS capillary electrophoresis.

| Molecule | Monomer [%] | Purity [%] |
|---|---|---|
| Anti-P329G (M-1.7.24) huIgG1 | 100 | 85 |
| Anti-P329G (VH1VL1) huIgG1 | 100 | 97 |
| Anti-P329G (VH2VL1) huIgG1 | 100 | 87 |
| Anti-P329G (VH3VL1) huIgG1 | 100 | 97 |

### Binding of Parental and Six Humanization Variants of Anti-P329G Binder M-1.7.24 to Human Fc (P329G)

Instrumentation: Biacore T200
Chip: CM5 (#772)
Fc1 to 4: anti-human Fab specific (GE Healthcare 28-9583-25)
Capture: 50 nM IgGs for 60 s
Analyte: human Fc (P329G) (P1AD9000-004)
Running buffer: HBS-EP
T°: 25° C.
Dilution: 2-fold dilution in HBS-EP from 0.59 to 37.5 nM
Flow: 30 μl/min
Association: 240 sec
Dissociation: 800 sec
Regeneration: 10 mM glycine pH 2.1 for 2×60 sec

SPR experiments were performed on a Biacore T200 with HBS-EP+ as running buffer (0.01 M HEPES pH 7.4, 0.15 M NaCl, 0.005% Surfactant P20 (BR-1006-69, GE Healthcare)). Anti-human Fab specific antibodies (GE Healthcare 28-9583-25) were directly immobilized by amine coupling on a CM5 chip (GE Healthcare). The IgGs were captured for 60 s at 50 nM. A two-fold dilution serie of the human Fc (P329G) was passed over the ligand at 30 μl/min for 240 sec to record the association phase. The dissociation phase was monitored for 800 s and triggered by switching from the sample solution to HBS-EP+. The chip surface was regenerated after every cycle using two injections of 10 mM glycine pH 2.1 for 60 sec. Bulk refractive index differences were corrected for by subtracting the response obtained on the reference flow cell 1. The affinity constants were derived from the kinetic rate constants by fitting to a 1:1 Langmuir binding using the Biaeval software (GE Healthcare). The measure was performed in triplicate with independent dilution series.

Following samples were analyzed for binding to human Fc (P329G) (Table 3).

TABLE 3
Description of the samples analyzed
for binding to human Fc (P329G).

| Binder | TAPIR ID | Format |
|---|---|---|
| Anti-P329G (M-1.7.24) | P1AE9963 | IgG, supernatant/purified |
| (parental) | | |
| Anti-P329G (VH3VL1) | P1AE9957 | IgG, supernatant/purified |
| Anti-P329G (VH1VL1) | P1AE9955 | IgG, supernatant/purified |
| Anti-P329G (VH2VL1) | P1AE9956 | IgG, supernatant/purified |
| Anti-P329G (VH4VL1) | P1AE9958 | IgG, supernatant |
| Anti-P329G (VH1VL2) | P1AE9959 | IgG, supernatant |
| Anti-P329G (VH1VL3) | P1AE9960 | IgG, supernatant |
| human Fc (P329G) | P1AD9000-004 | Antigen used as analyte |

Human Fc (P329G) was prepared by plasmin digestion of a human IgG1 followed by affinity purification by ProteinA and size exclusion chromatography.

### Binding of Parental and Six Humanization Variants of Anti-P329G Binder M-1.7.24 to Human Fc (P329G)

The dissociation phase was fitted to a single curve to help characterize the off-rate. The ratio between binding to capture response level was calculated. (Table 4).

TABLE 4
Binding assessment of six humanization variants
for binding to human Fc (P329G).

| | | | Ratio | |
|---|---|---|---|---|
| Binder | TAPIR ID | kd (1/s) | binding/capture | Binding |
| Anti-P329G | P1AE9963-001 | 5.73E−03 | 20 | parental |
| (M-1.7.24) | | | | |
| (parental) | | | | |
| Anti-P329G | P1AE9957-001 | 5.49E−03 | 20 | as parental |
| (VH3VL1) | | | | |
| Anti-P329G | P1AE9955-001 | 3.88E−03 | 20 | as parental |
| (VH1VL1) | | | | |
| Anti-P329G | P1AE9956-001 | 2.79E−03 | 23 | as parental |
| (VH2VL1) | | | | |
| Anti-P329G | P1AE9958-001 | 1.11E−02 | 19 | reduced |
| (VH4VL1) | | | | |
| Anti-P329G | P1AE9959-001 | 7.86E−03 | 10 | reduced |
| (VH1VL2) | | | | |
| Anti-P329G | P1AE9960-001 | 1.29E−01 | 3 | reduced |
| (VH1VL3) | | | | |

### Affinity of Parental and Three Humanization Variants of Anti-P329G Binder M-1.7.24 to Human Fc (P329G)

Three humanization variants with binding pattern similar to parental were assessed in more details. The kinetic constants for a 1:1 Langmuir binding are summarized in Table 5.

TABLE 5
Kinetic constants (1:1 Langmuir binding). Average and standard
deviation (in parenthesis) of independent triplicate (independent
dilutions series within the same run).

| Binder | TAPIR ID | ka (1/Ms) | kd (1/s) | KD (M) | Rmax (RU) |
|---|---|---|---|---|---|
| Anti-P329G | P1AE9963-003 | 5.03E+05 | 1.58E−03 | 3.17E−09 | 44 |
| (M-1.7.24) | | (4.75E+04) | (3.8E−05) | (3.7E−10) | (2) |
| (parental) | | | | | |
| Anti-P329G | P1AE9957-003 | 2.74E+05 | 1.44E−03 | 5.27E−09 | 55 |
| (VH3VL1) | | (5.51E+03) | (7.51E−05) | (3.3E−10) | (3) |
| Anti-P329G | P1AE9955-003 | 2.83E+05 | 1.20E−03 | 4.24E−09 | 48 |
| (VH1VL1) | | (7.94E+03) | (4.73E−05) | (2.5E−10) | (2) |
| Anti-P329G | P1AE9956-003 | 2.53E+05 | 1.22E−03 | 4.81E−09 | 54 |
| (VH2VL1) | | (3.79E+03) | (3.61E−05) | (2.1E−10) | (5) |

### Conclusion

Six humanization variants were generated. Three of them (VH4VL1, VH1VL2, VH1VL3) showed decreased binding to human Fc (P329G) compared to parental M-1.7.24. The other three humanization variants (VH1VL1, VH2VL1, VH3VL1) have a binding kinetic very similar to the parental binder and did not lose affinity through humanization.

### Example 2

### Preparation of Humanized Anti-P329G Antigen Binding Receptors

To assess the functionality of the humanized P329G variants the different variable domains of heavy (VH) and light chain (VL) DNA sequences encoding a binder specific for the P329G Fc mutation were cloned as single chain variable Fragment (scFv) binding moieties and employed as antigen binding domain in a second generation chimeric antigen receptor (CAR).

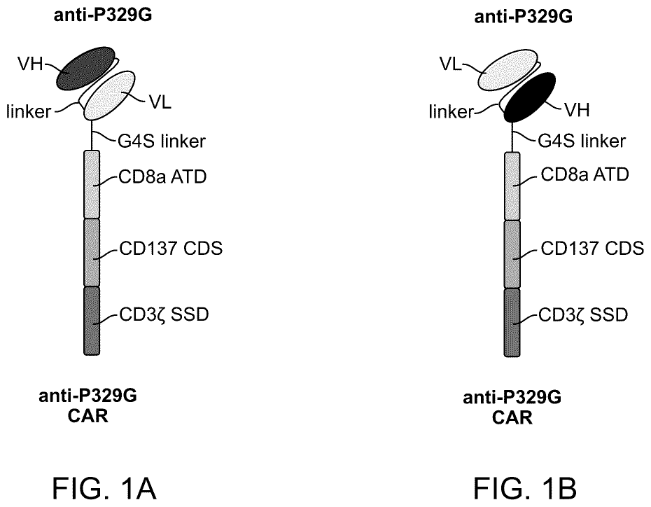

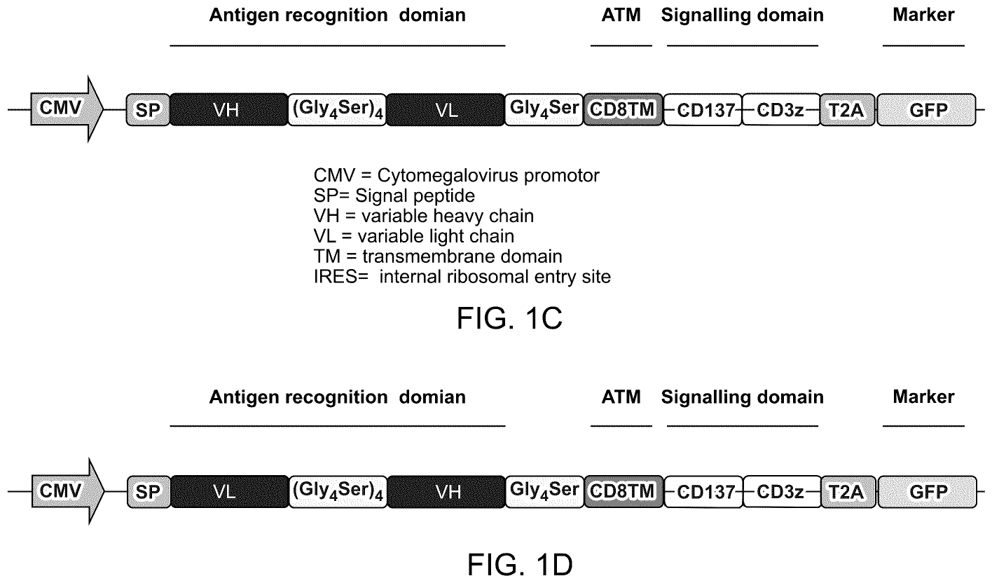

The different humanized variants of the P329G binder comprise an Ig heavy chain variable main domain (VL) and an Ig light chain variable domain (VL). VH and VL are connected via (G4S)4 linker. The scFv antigen binding domain was fused to the anchoring transmembrane domain (ATD) CD8a (Uniprot P01732[183-203]), which is fused to an intracellular co-stimulatory signalling domain (CSD) CD137 (Uniprot Q0701 1AA 214-255), which in turn is fused to a stimulatory signalling domain (SSD) CD3ζ (Uniprot P20963 AA 52-164). The scFv of the anti-P329G CAR was constructed in two different orientations VHxVL (FIG. 1A) or VLxVH (FIG. 1i). A graphical representation of an exemplary expression construct (including the GFP reporter) for the VHVL configuration is shown in FIG. 1C and for the VLVH configuration in FIG. 1D.

### Example 3

### Expression of Anti-P329G Antigen Binding Receptors in Jurkat-NFAT Cells

The different humanized anti-P329G antigen binding receptors were virally transduced into Jurkat (GloResponse Jurkat NFAT-RE-luc2P, Promega #CS176501) cells.

The anti-P329G antigen binding receptor expression was assess via flow cytometry. Jurkat cells employing different humanized anti-P329G antigen binding receptors were harvested, washed with PBS and seeded at 50.000 cells per well in a 96 well flat bottom plate. After staining for 45 min in the dark and the fridge (4-8° C.) with different concentrations (500 nM-0 nM serial dilution of 1:5) of antibody comprising the P329G mutation in the Fc domain, samples were washed three times with FACS-buffer (PBS containing 2% FBS, 10% 0.5 M EDTA, pH 8 and 0.5 g/L NaN3)). Samples were then stained with 2.5 μg/mL polyclonal anti-human IgG Fcγ fragment-specific and PE-conjugated AffiniPure F (ab′)2 goat fragment antibody for 30 min in the dark in the fridge analyzed with flow cytometry (Fortessa BD). Additionally, the anti-P329G antigen binding receptors comprised an intracellular GFP reporter (see FIG. 1C).

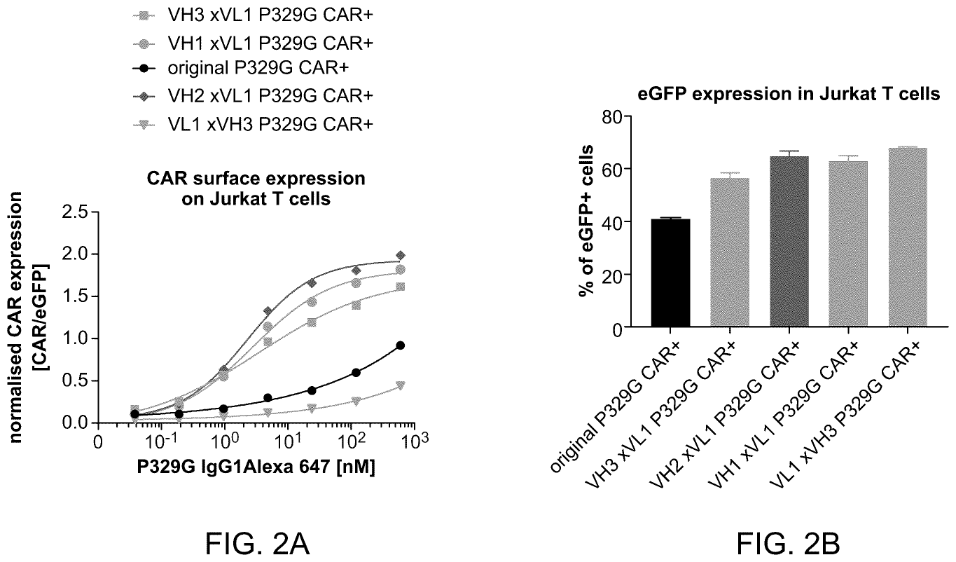

Compared to the humanized versions (VH1VL1, VH2VL1 and VH3VL1) of the P329G binder the original non-humanized binder shows weak CAR-labeling on the cell surface (FIG. 2A), although the GFP expression is comparable. Interestingly, the VL1VH1 construct (see FIG. 1D) shows a high GFP expression but also weak CAR-labeling on the cell surface, indicating that this is a non-favorable confirmation of the binder.

Overall, unexpectedly, the VH3VL1 version shows the highest GFP expression and CAR surface expression. Furthermore, all tested constructs in the VHVL confirmation (VH1VL1, VH2VL1 and VH3VL1) show enhanced GFP signal upon transduction into Jurkat T cells compared to the original non-humanized P329G antigen binding receptor and, interestingly, the construct in the VLVH confirmation (VL1VH3).

In conclusion, the VHVL confirmation seems to favor expression levels of the antigen binding receptors as well as correct targeting to the cell surface.

To further, characterise the selectivity, specificity and safety of the humanised anti-P329G antigen binding receptors different tests were conducted.

### Example 4

### Specific T Cell Activation in the Presence of Targeting Antibody Comprising the P329G Mutation in the Fc Domain

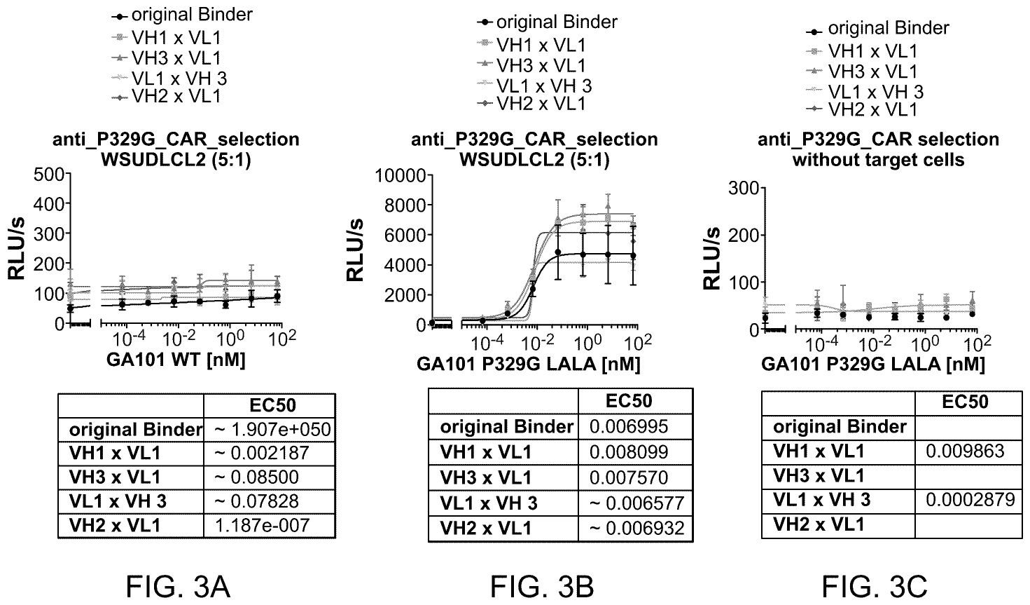

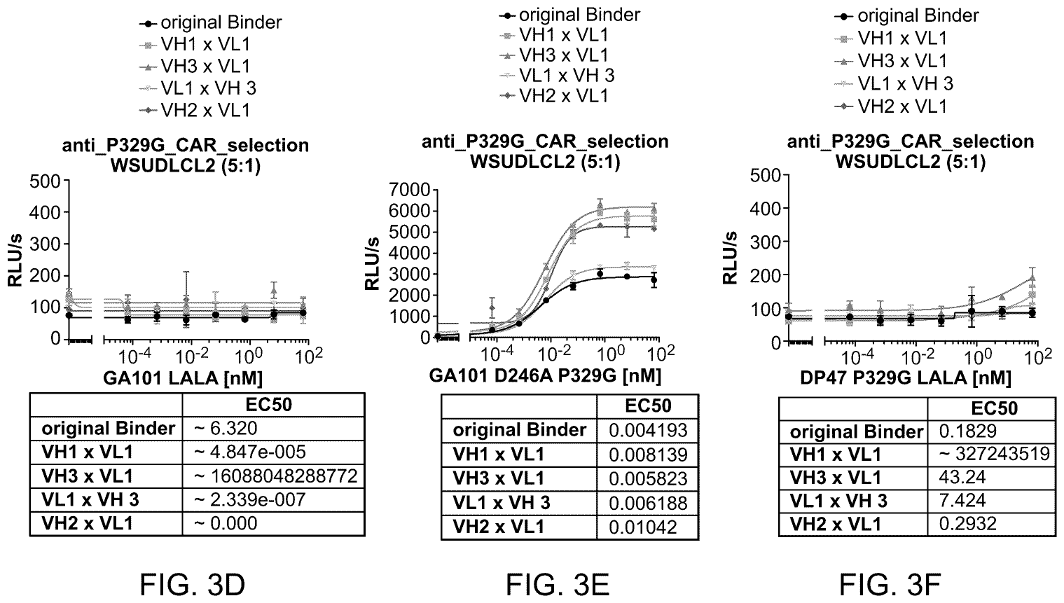

To exclude unspecific binding of the different humanised anti-P329G-scFv variants, Jurkat NFAT cells expressing the antigen binding receptors comprising these variants were evaluated towards their activation in the presence of CD20-positive WSUDLCL2 target cells and anti-CD20 (GA101) antibodies with different Fc variants (Fc wildtype, Fc P329G mutation, LALA mutation, D246A mutation or combinations thereof). The CAR-Jurkat NFAT activation assay was performed as described above and the anti-CD20 (GA101) wild type IgG1 (FIG. 3A), anti-CD20 (GA101) P329G LALA IgG1 (FIG. 3B), anti-CD20 (GA101) LALA IgG1 (FIG. 3D), anti-CD20 (GA101) D246A P329G IgG1 (FIG. 3F) or a non-specific DP-47 P329G LALA IgG1 (FIG. 3E) were used to evaluate the potential of unspecific binding. No unspecific anti-P329G CAR activation could be detected for anti-CD20 (GA101) wild type IgG1 (FIG. 3A), anti-CD20 (GA101) LALA IgG1 (FIG. 3D) or the non-specific DP-47 P329G LALA IgG1 (FIG. 3E).

Specific anti-P329G CAR activation could be detected in the presence of anti-CD20 (GA101) P329G LALA IgG1 (FIG. 3B) and anti-CD20 (GA101) D246A P329G IgG1 (FIG. 3F). The assessed EC₅₀ was comparable between all humanised anti-P329G variants and did not differ from the EC₅₀ of the original binder.

Interestingly, the antigen binding receptors comprising scFv binders in the VHVL conformation lead to stronger activation of the Jurkat NFAT T cells compared to the original non-humanized binder and the humanized binder in the VLVH conformation. The higher plateau (see for example FIG. 3F) could be due to the improved expression levels and/or improved transport to the cell surface of the antigen binding receptors resulting in a stronger activation. Furthermore, the conformation could have an impact on binding to the P329G mutation.

To investigate the risk of potential antigen binding domain clustering, resulting in tonic signalling or unspecific activation of the T cells, the Jurkat NFAT activation assay was performed as described above whereas the initial antibody concentration used was elevated and the serial dilution was started with 100 nM of GA101 P329G LALA IgG1 and further no target cells were seeded.

As depicted in FIG. 3C, no activation was detectable for all tested humanised P329G variants, indicating detectable receptor clustering or unspecific activation in the absence of target cells.

### Example 5

### Sensitivity of Different Humanized P329G Antigen Binding Receptor Variants Assessed by T Cell Activation on Target Cells Expressing Different Levels of Antigen

To further, characterise the sensitivity and selectivity of the humanised anti-P329G antigen binding receptors the Jurkat NFAT activation assay was performed as described above.

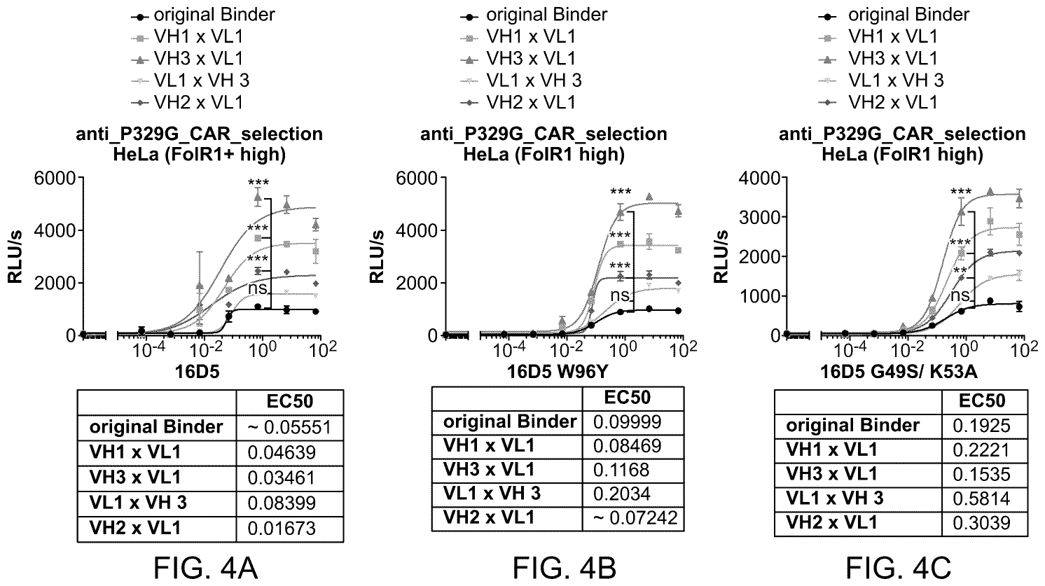

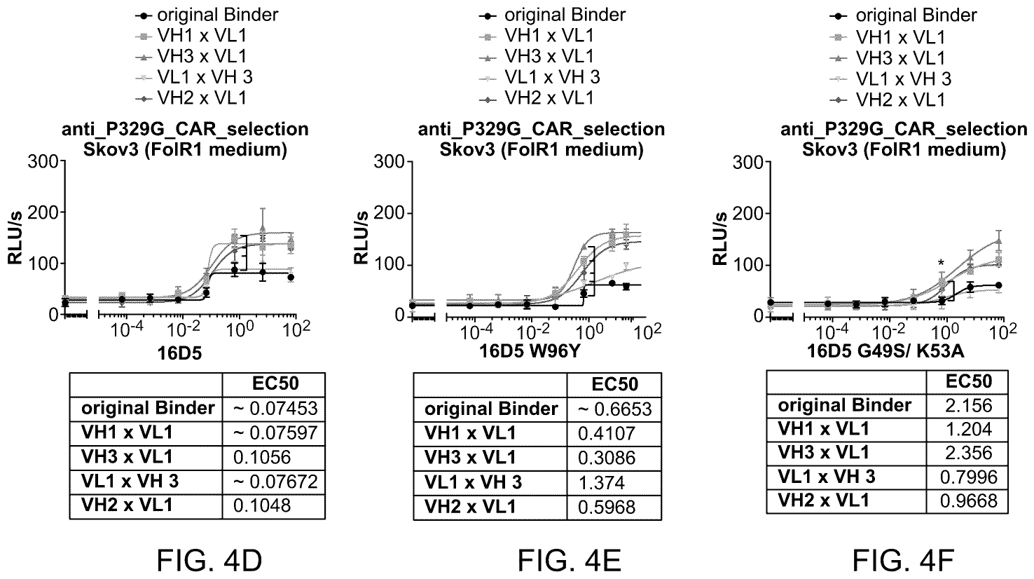

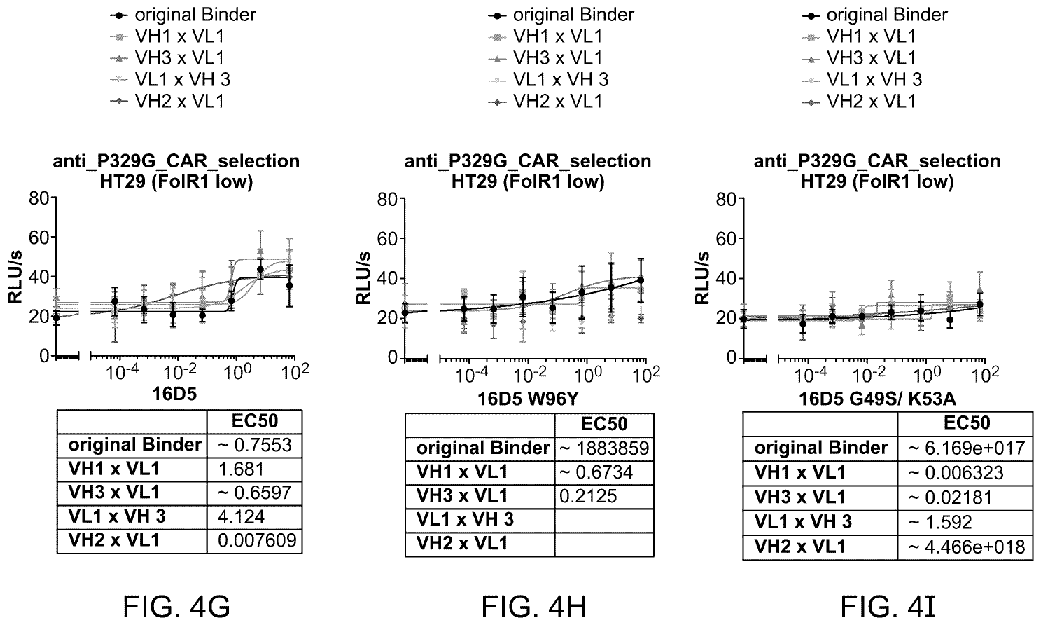

The Jurkat NFAT reporter cells expressing the different humanised anti-P329G-scFv variant antigen binding receptors were evaluated towards their ability to discriminate between high (HeLa-FolR1), medium (Skov3) and low (HT29) FolR1-positive target cells. Different variants of the anti-P329G binder were used as scFv antigen recognition scaffold in the Jurkat-Reporter cell line in combination with antibodies that poses high (16D5) (FIGS. 4A, D, G), medium (16D5 W96Y) (FIGS. 4B, E, H) or low (16D5 G49S/K53A) (FIGS. 4C, F, I) affinities towards FolR1. High expressing target cells HeLa-FolR1, combined with high anti-FolR1 16D5 (FIG. 4A), medium anti-FolR1 16D5 W96Y (FIG. 4B) and low affinity Adapter-IgG anti-FolR1 G49S K53A (FIG. 4C) showed a dose dependent activation. Medium expressing target cells Skov3, combined with high anti-FolR1 16D5 (FIG. 4D), medium anti-FolR1 16D5 W96Y (FIG. 4E) and low affinity adaptor-IgG anti-FolR1 G49S K53A (FIG. 4F) showed a dose dependent activation. For low expressing target cells HT29, combined with the different affinity binder anti-FolR1 16D5 (FIG. 4G), anti-FolR1 16D5 W96Y (FIG. 4H) or low affinity Adaptor-IgG anti-FolR1 G49S K53A (FIG. 4I), no signal could be detected. Further, interestingly, the antigen binding receptors in the VHVL format result with higher activation of the Jurkat NFAT T cells compared to the original non-humanized binder and the humanized binder in the VLVH format. The humanised variant VH3VL1 scFv binder results with the highest signal intensity of all constructs (FIGS. 4A-F).

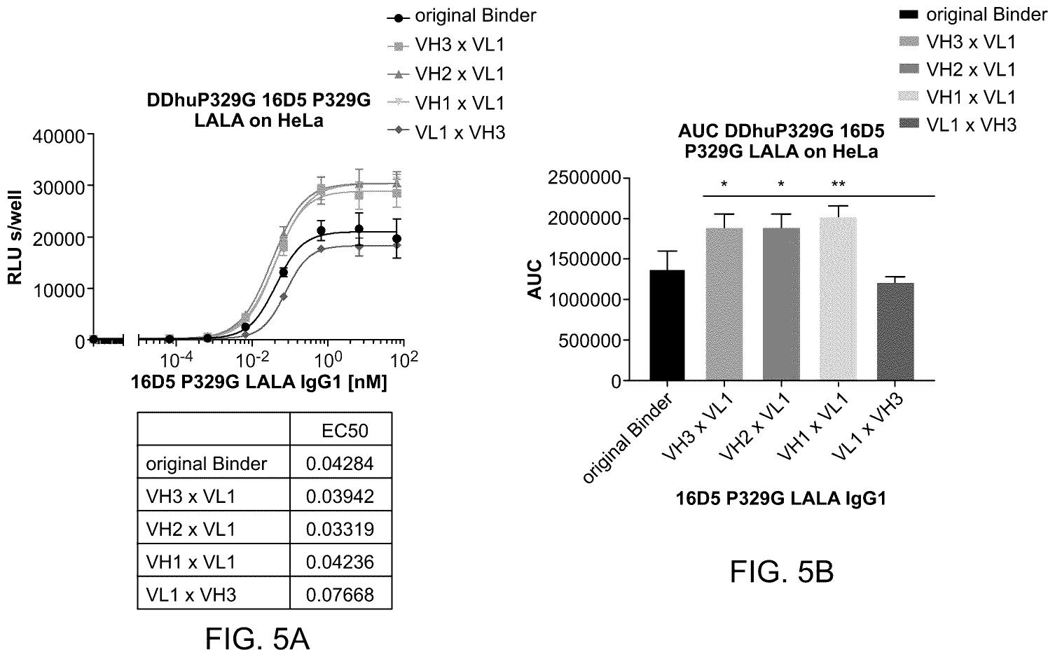

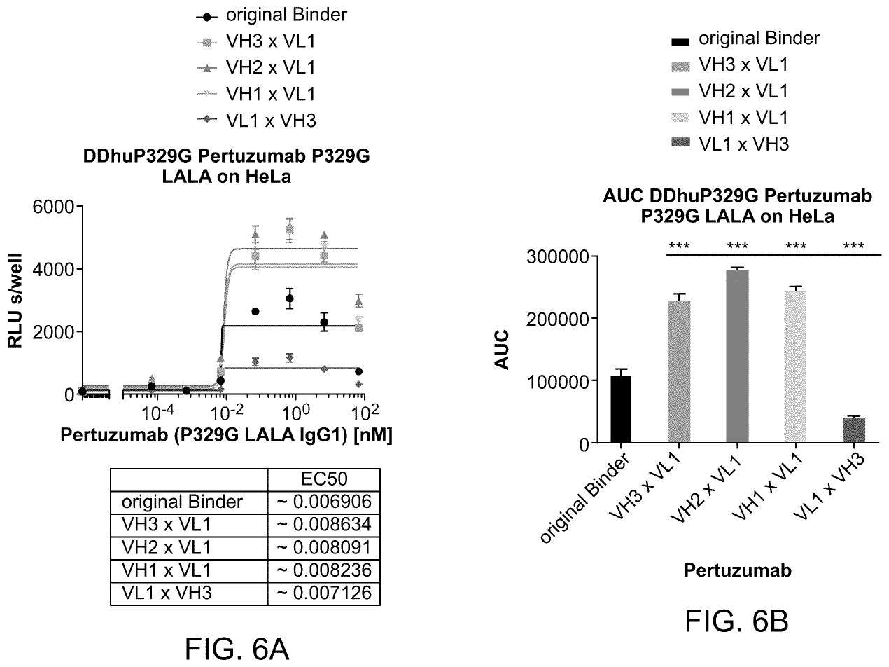

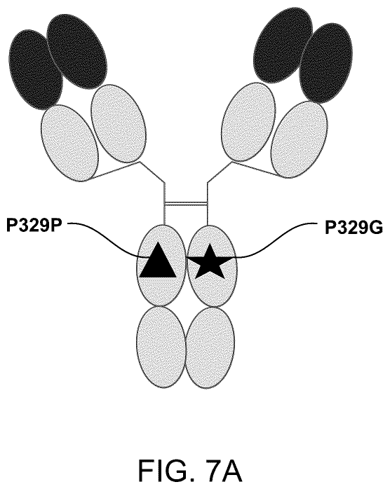

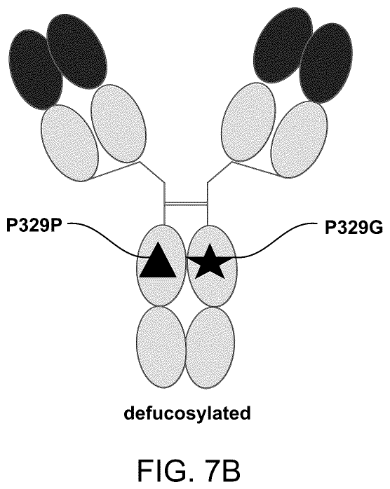

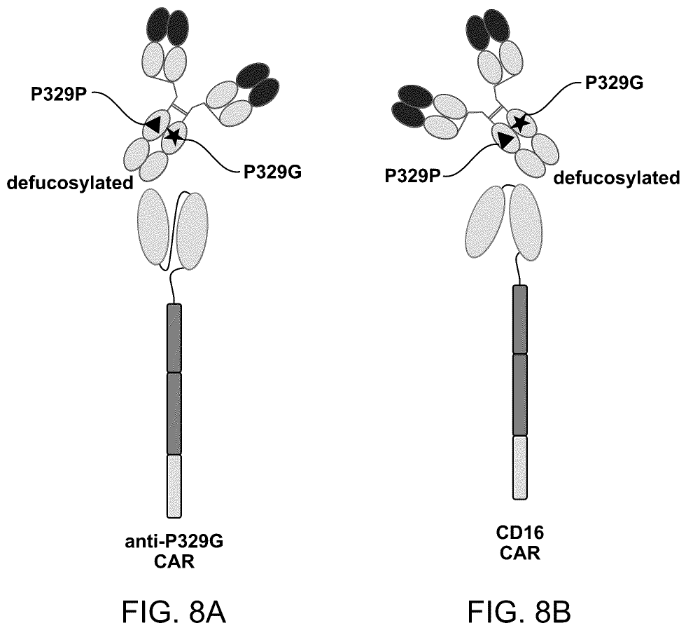

Further, the Jurkat NFAT activation assay was performed on HeLa (FolR1⁺ and HER2⁺) cells used in combination with either anti-FolR1 16D5 P329G LALA IgG1 (FIG. 5) or anti-HER2 P329G LALA IgG1 (FIG. 6). Both confirm the finding that the VHVL orientation is superior compared to the VLVH orientation. The humanised variant VH3VL1 leads to the strongest activation of the Jurkat NFAT T cells.

### Example 6

### Specific T Cell Activation in the Presence of Heterodimeric Targeting Antibody Comprising the P329G Mutation in One Subunit of the Fc Domain

The ability of the heterodimeric anti-CD20 IgG (SEQ ID Nos: 129-131) to selectively recruit anti-P329G CAR (SEQ ID NO: 7) Jurkat reporter T cells or CD16-CAR Jurkat reporter T cells was assessed by a co-culture of the respective Jurkat reporter T cells and WSUDLCL2 (CD20+) target cells. The CAR-Jurkat NFAT activation assay was performed as described above and the anti-CD20 (GA101) wild type IgG1, anti-CD20 (GA101) P329G LALA IgG1, anti-CD20 (GA101) defucosylated IgG1 and the anti-CD20 (GA101) heterodimeric IgG were titrated. For the CD16-CAR Jurkat NFAT T cells a specific, dose-dependent activation could be observed if anti-CD20 (GA101) wildtype IgG1, anti-CD20 (GA101) defucosylated IgG1 or the anti-CD20 (GA101) heterodimeric IgG1 was used (FIG. 9A). For the anti-P329G CAR Jurkat T cells a specific, dose-dependent activation could be observed if anti-CD20 (GA101) P329G LALA IgG1 or the anti-CD20 (GA101) heterodimeric IgG1 was used (FIG. 9B).

### Example 7

Specific Target Cell Lyses by CD16-CAR T Cells Recruited with the Heterodimeric IgG1

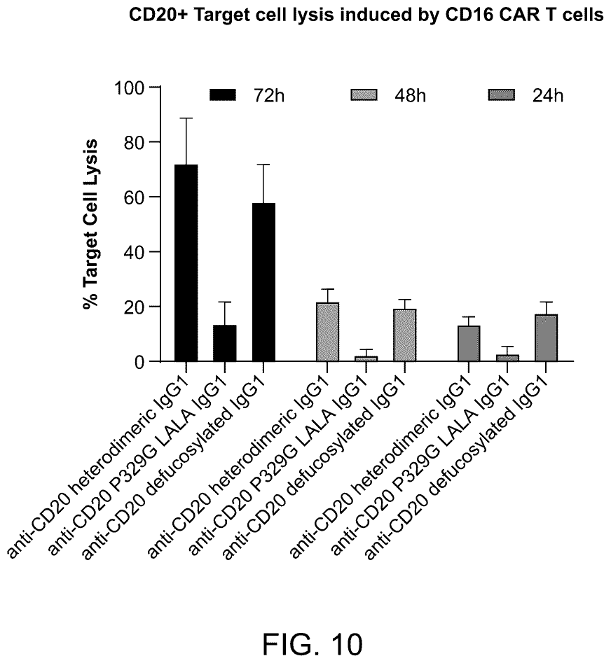

The ability of the heterodimeric IgG to selectively recruit CD16-CAR T cells and induce tumor cell lysis was assessed by a co-culture of the respective Jurkat reporter T cells and WSUDLCL2 (CD20+) target cells. The CAR-Jurkat NFAT activation assay was performed as described above and the anti-CD20 (GA101) wild type IgG1, anti-CD20 (GA101) P329G LALA IgG1, anti-CD20 (GA101) defucoslyated IgG1 and the anti-CD20 (GA101) heterodimeric IgG were titrated. For the CD16-CAR Jurkat NFAT T cells a specific, dose-dependent activation could be observed if anti-CD20 (GA101) P329G LALA IgG1 or the anti-CD20 (GA101) heterodimeric IgG1 was used (FIG. 10A). For the anti-P329G CAR Jurkat T cells a specific, dose-dependent activation could be observed if anti-CD20 (GA101) wildtype IgG1, anti-CD20 (GA101) defucosylated IgG1 or the anti-CD20 (GA101) heterodimeric IgG1 was used (FIG. 10B).

### Example 8

### Ability of the Heterodimeric IgG1 to Induce ADCC

To asses the ability of the heterodimeric IgG1 to induce ADCC, the antibody was titrated into a co-culture of PBMCs from healthy donors and WSUDLCLS (CD20+). After 4.5 h LDH release was measured. In order to perform the assay PBMCs were isolated via density gradient centrifugation using Histopaque-1077 (Sigma). 50 μl/well (0.625 Mio cells/well) of the isolated PBMCs were seeded into 96-round bottom well plate. WSUDLCL2 Target cells were harvested counted and checked for their viability, 0.025 Mio cells/well were seeded in 50 μl/well onto the PBMCs. Different concentrations of either anti-CD20 (GA101) heterodimeric IgG1, anti-CD20 (GA101) defucosylated, anti-CD20 (GA101) wildtype IgG1 or anti-CD20 (GA101) P329G LALA were added. Cells were stained directly with anti-CD107a-PE and incubated for 4.5 h in the incubator at 37° C., 5% CO2 and humidified atmosphere. 1 h before the readout 50 μl/well of 4% Triton X-100 were added to the maximal release wells (target cells only). After the final incubation time 50 μl supernatant were transferred into a flat bottom TPP plate and 50 μl of LDH-substrate (LDH-kit; Roche), prepared according to the manufacturers instructions were added. Absorbance was measured immediately at the Tecan-reader (490 nm-650 nm) for 10 min.

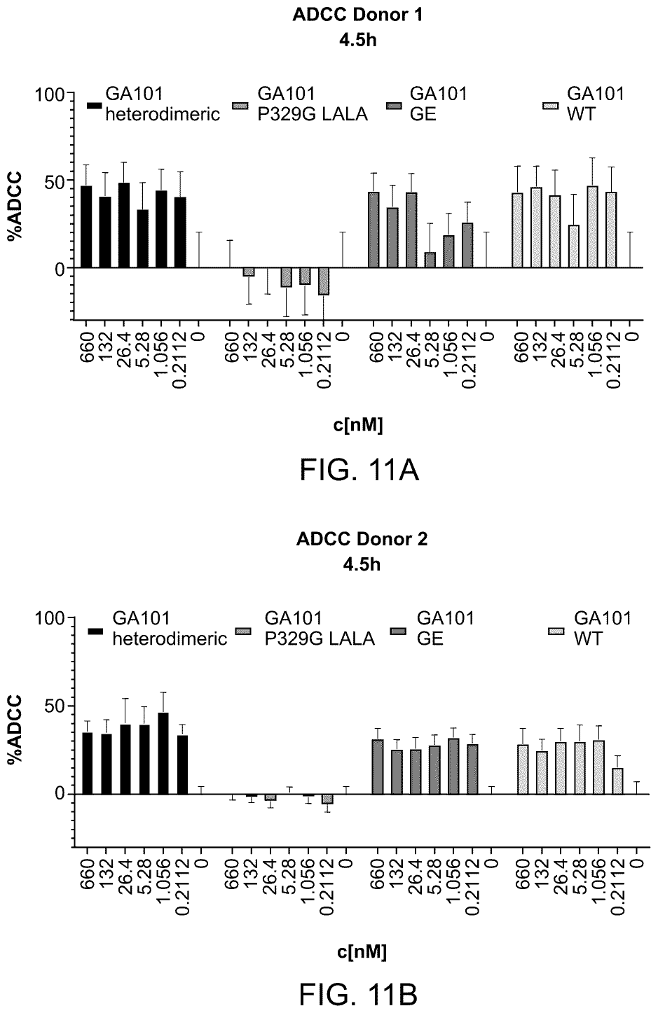

Bar diagram depicts the mean calculated from technical triplicates. Interestingly it could be shown, that that the heterodimeric IgG was able to induce ADCC to the same extend then the defucosylated IgG1 variant (FIGS. 11A and 11B).

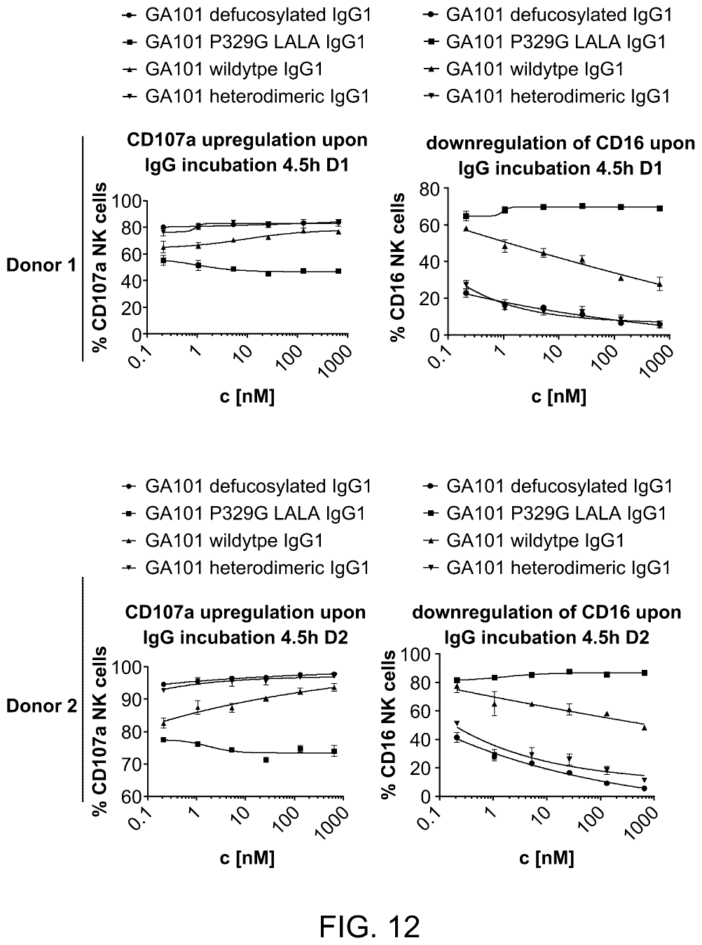

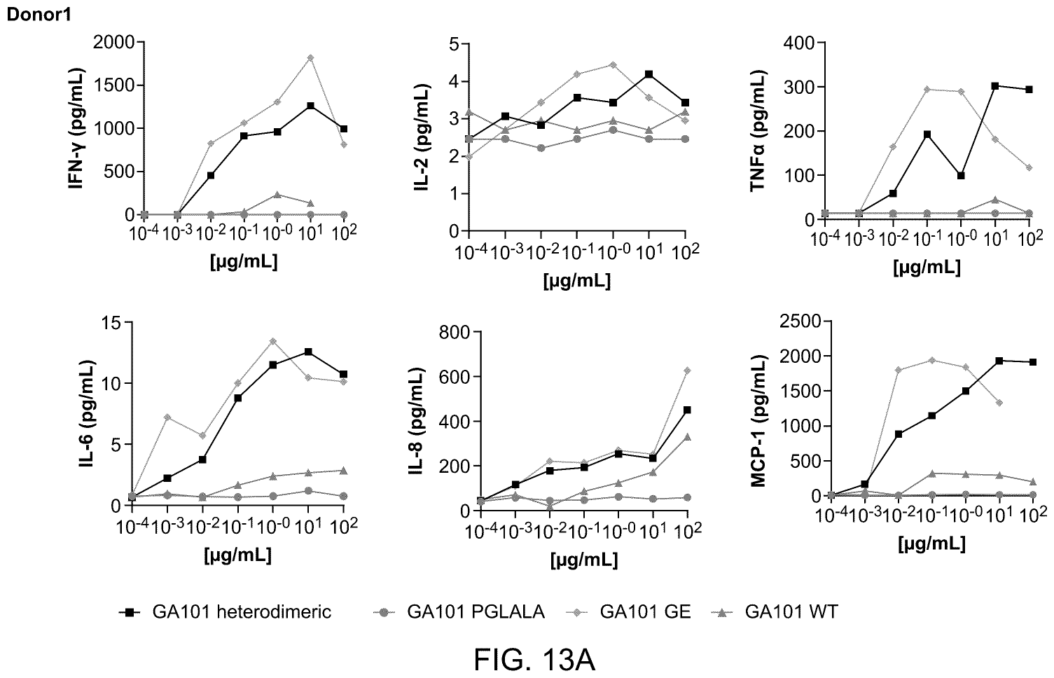

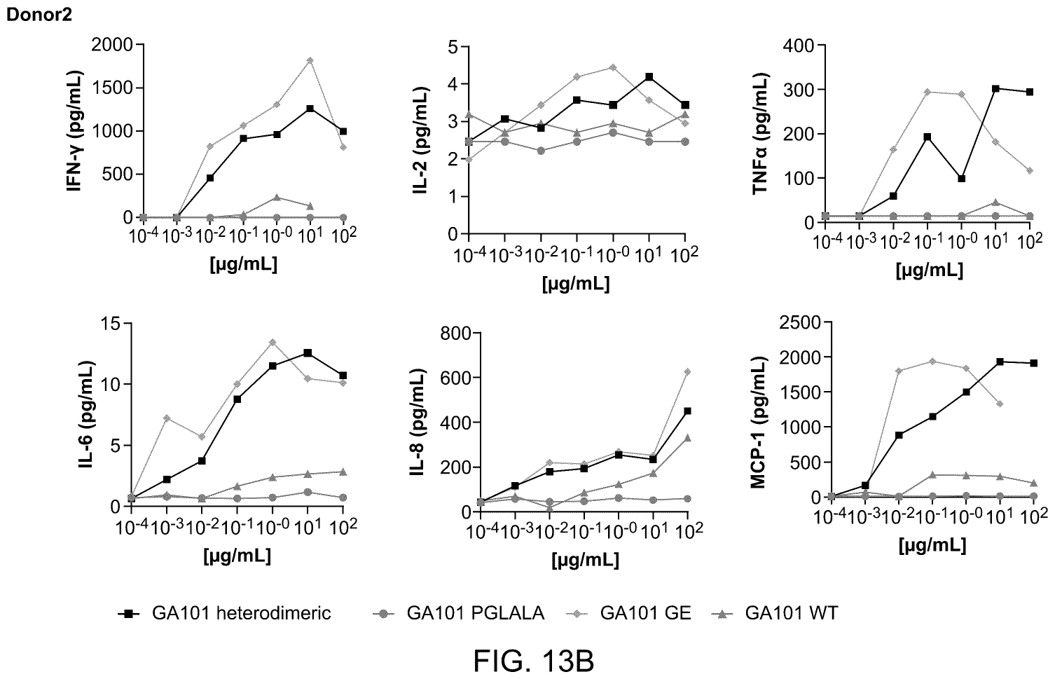

To evaluate the NK cell activation during that assay, the remaining cells were used for FACS analysis. Therefore cells were washed two times in PBS and stained with 50 μl/well for 30 min at 4° C. in the dark. The FACS ab staining mix 400 μl CD3-PE/Cy7+400 μl CD56-APC+400 μl CD16-FITC+18800 μl PBS buffer+eF 450 as live dead staining. After the staining cells were washed 3 times with FACS buffer. Samples were acquired in a final volume of 150 μl at FACS Fortessa (FACSDiva software). The activation of NK cells in the presence of anti-CD20 heterodimeric IgG1, anti-CD20 P329G LALA IgG, anti-CD20 defucosylated IgG1 and the wildtype IgG1 was assessed. The activation of NK cells could be demonstrated by upregulation of CD107a and downregulation of CD16 receptor in the presence of the defucosylated variant, the heterodimeric variant and the wildtype variant (FIGS. 12A and 12B). Interestingly the heterodimeric IgG activated the NK cells to the same extend then the defucosylated IgG1.

### Example 9

### Effect of the Different Fc Variants of Anti-CD20 Antibodies on Cytokine Release and B Cell Depletion.

To assess the effect of the different Fc variants (Fc wildtype, Fc P329G mutation, defucosylated or a combination of defucosylation and P329G mutation) of the anti-CD20 antibody (GA101) on B cell depletion and cytokine release, escalating concentrations of the different anti-CD20 antibodies were incubated in fresh whole blood. After 24 h, the serum was collected for cytokine measurement using the Luminex technology. After 48 h, the percentages of CD19+ B cells among CD45+ cells were measured by flow cytometry.

The levels of IFN-7, TNF-α, IL-2, IL-6, IL-8 and MCP-1 were comparable for the GE GA101 (defucosylated Fc) and the heterodimeric GA101 (P329G and defucosylation) and higher than observed for WT GA101 (wild type Fc) and PGLALA GA101 (P329GLALA mutation) for donor 1 (FIG. 14A) and donor 2 (FIG. 14B). This indicates that the activity of the heterodimeric GA101 and the defucoslyated GA101 are comparable in terms of cytokine release. In addition, the percentage of CD19+ B cells among CD45+ cells is comparable for the defucosylated GA101 and the heterodimeric GA101 and lower than observed for the wildtype GA101 or P329G LALA GA101 (not shown). The percentage of CD19+ B cells among CD45+ cells was much higher for the P329G LALA GA101 as opposed to wild type GA101, defucosylated GA101. As expected, that the P329G LALA mutation leads to a much lower activity of the anti-CD20 antibody in terms of B cell depletion. This data suggests that the combination of defucosylation and P329G LALA mutation on the Fc of anti-CD20 antibodies leads to comparable activity than defucosylation alone and superior activity than wildtype GA101.

Overall, this experiment suggests that the heterodimeric does not impair B cell depletion and cytokine release. Further it leads to enhanced B cell depletion and cytokine release as opposed to WT GA101 (wild type Fc) or PGLALA GA101 (Fc P329G, LALA mutation).

1. An antibody comprising a heterodimeric Fc domain composed of a first and a second subunit, wherein the first subunit comprises the amino acid mutation P329G according to EU numbering and wherein the second subunit comprises a proline (P) at position 329 according to EU numbering.

2. The antibody of claim 1, wherein the Fc domain is an IgG, particularly an IgG₁, Fc domain.

3. The antibody claim 1, wherein the Fc domain is a human Fc domain.

4. The antibody of claim 1, wherein the Fc domain comprises a modification promoting the association of the first and the second subunit of the Fc domain.

5. The antibody of claim 1, wherein the antibody is defucosylated.

6. The antibody of claim 1, wherein the heterodimeric Fc domain exhibits increased binding affinity to an Fc receptor and/or increased effector function, as compared to a native IgG₁ Fc domain, in particular wherein the effector function is ADCC.

7. The antibody of claim 1, wherein the heterodimeric Fc domain comprises one or more amino acid mutations that increase binding to an Fc receptor and/or effector function, in particular wherein the effector function is ADCC.

8. The antibody of claim 1, wherein the antibody comprises at least one antigen binding moiety capable of specific binding to an antigen on a target cell.

9. The antibody of claim 1, wherein the target cell is a cancer cell.

10. The antibody of claim 1, wherein the antigen is selected from the group consisting of FAP, CEA, p95 HER2, BCMA, EpCAM, MSLN, MCSP, HER-1, HER-2, HER-3, CD19, CD20, CD22, CD33, CD38, CD52Flt3, EpCAM, IGF-1R, FOLR1, Trop-2, CA-12-5, HLA-DR, MUC-1 (mucin), GD2, A33-antigen, PSMA, PSCA, transferrin-receptor, TNC (tenascin) and CA-IX.

11. The antibody of claim 8, wherein the antigen binding moiety is a scFv, a Fab, a crossFab or a scFab.

12. The antibody of claim 1, which is a human, humanized or chimeric antibody.

13. The antibody of claim 1, wherein the antibody is a multispecific antibody.

14. An isolated polynucleotide encoding the antibody of claim 1.

15. A host cell comprising the isolated polynucleotide of claim 14.

16. A method of producing an antibody, comprising the steps of (a) culturing the host cell of claim 15 under conditions suitable for the expression of the antibody and optionally (b) recovering the antibody.

17. (canceled)

18. A pharmaceutical composition comprising the antibody of claim 1 and a pharmaceutically acceptable carrier.

19-23. (canceled)

24. A method of treating or delaying progression of a cancer in an individual comprising administering to said individual an effective amount of an antibody and a transduced T cell, wherein the antibody comprises a heterodimeric Fc domain composed of a first and a second subunit, wherein the first subunit comprises the amino acid mutation P329G according to EU numbering, wherein the second subunit comprises a proline (P) at position 329 according to EU numbering, and wherein the transduced T cell expresses an antigen binding receptor capable of specific binding to the first subunit.

25. The method of claim 24, wherein the antigen binding receptor is capable of specific binding to an Fc domain subunit comprising the amino acid mutation P329G according to EU numbering.

26. The method of claim 24, wherein the antigen binding receptor comprises a heavy chain variable domain (VH) comprising:
(a) a heavy chain complementarity determining region (CDR H) 1 amino acid sequence of RYWMN (SEQ ID NO:1);
(b) a CDR H2 amino acid sequence of EITPDSSTINYAPSLKG (SEQ ID NO:2) or of EITPDSSTINYTPSLKG (SEQ ID NO:40);
(c) a CDR H3 amino acid sequence of PYDYGAWFAS (SEQ ID NO:3); and a light chain variable domain (VL) comprising:
(d) a light chain (CDR L)1 amino acid sequence of RSSTGAVTTSNYAN (SEQ ID NO:4);
(e) a CDR L2 amino acid sequence of GTNKRAP (SEQ ID NO:5); and
(f) a CDR L3 amino acid sequence of ALWYSNHWV (SEQ ID NO:6).

27. The method of claim 24, wherein the antigen binding receptor comprises:
(i) a transmembrane domain selected from the group consisting of the CD8, the CD3z, the FCGR3A, the NKG2D, the CD27, the CD28, the CD137, the OX40, the ICOS, the DAP10 or the DAP12 transmembrane domain or a fragment thereof, in particular the CD28 transmembrane domain or a fragment thereof,
(ii) at least one stimulatory signaling domain selected from the group consisting of the intracellular domain of CD3z, of FCGR3A and of NKG2D, or fragments thereof, in particular wherein the at least one stimulatory signaling domain is the CD3z intracellular domain or a fragment thereof, and/or
(iii) at least one co-stimulatory signaling domain individually selected from the group consisting of the intracellular domain of CD27, of CD28, of CD137, of OX40, of ICOS, of DAP10 and of DAP12, or fragments thereof, in particular wherein the at least one co-stimulatory signaling domain is the CD28 intracellular domain or a fragment thereof.

28. The method of claim 24, wherein the transduced T cell is administered before, simultaneously with or after administration of the antibody.

29-33. (canceled)

34. A kit comprising:
(a) an antibody comprising a heterodimeric Fc domain composed of a first and a second subunit, wherein the first subunit comprises the amino acid mutation P329G according to EU numbering, wherein the second subunit comprises a proline (P) at position 329 according to EU numbering.
(b) (b) a transduced T cell capable of expressing an antigen binding receptor capable of specific binding to the first subunit.

35. A kit comprising:
(a) an antibody comprising a heterodimeric Fc domain composed of a first and a second subunit, wherein the first subunit comprises the amino acid mutation P329G according to EU numbering, wherein the second subunit comprises a proline (P) at position 329 according to EU numbering.
(b) (b) an isolated polynucleotide encoding an antigen binding receptor capable of specific binding to the first subunit.

36. (canceled)
